(19) United States
(12) Patent Application Publication
(10) Pub. No.: US 2025/0197548 A1
(43) Pub. Date: Jun. 19, 2025

(54) BIODEGRADABLE GRAFT POLYMERS

(71) Applicant: BASF SE, Ludwigshafen am Rhein (DE)

(72) Inventors: Stephan HUEFFER, Ludwigshafen (DE); Thomas WEISS, Ludwigshafen (DE); Peter OECHSLE, Ludwigshafen (DE); Verena MORMUL, Ludwigshafen (DE); Erik BARTHEL, Ludwigshafen (DE); Stefan LAST, Ludwigshafen (DE); Aaron WAGNER, Ludwigshafen (DE); Susanne WOLWERTZ, Ludwigshafen (DE); Janine RUDE, Ludwigshafen (DE); Joachim BENTELE, Ludwigshafen (DE); Doris KREMZOW-GRAW, Ludwigshafen (DE)

(21) Appl. No.: 18/562,061

(22) PCT Filed: Jun. 13, 2022
(86) PCT No.: PCT/EP22/65983

§ 371 Date: Nov. 17, 2023

(30) Foreign Application Priority Data
Jun. 18, 2021 (EP) 21180319.2
Jun. 29, 2021 (EP) 21182316.6

(51) Int. Cl. C08F 283/02; A01N 25/10; A01P 3/00; A01P 7/02; A01P 7/04; C08G 63/664; C08G 63/672; C11D 3/37

(52) U.S. Cl. CPC C08F 283/02; A01N 25/10; A01P 3/00; A01P 7/02; A01P 7/04; C08G 63/664; C08G 63/672; C11D 3/3788

(57) ABSTRACT

Disclosed herein are graft polymers, their preparation, and methods of using graft polymers, where the graft polymers include as polymer backbone (A) a polyalkylene oxide ester polymer with a weight average molecular weight Mw of 500 to 50 000 g/mol and a polydispersity PD of 2 to 6, including 10 to 560 ether groups and 2 to 51 ester groups, which are interconnected with alkylene groups, which is significantly more biodegradable than conventional polyalkylene oxide polymers, and polymeric side chains grafted onto the polymer backbone A, where the polymeric sidechains (B) are obtainable by polymerization of at least one monomer being selected from i) at least one vinyl ester monomer (B1) and ii) optionally at least one further olefinically unsaturated monomer (B2) polymerizable with monomer B1.

The present invention relates to novel graft polymers comprising a polymer backbone (A) as a graft base having polymeric sidechains (B) grafted thereon.

The polymer backbone (A) is a polyalkylene oxide ester polymer (“PAG-ester” polymer) comprising as building blocks i) diols of polyalkylene oxides (“PAG”), ii) di-carbonic acids of PAG and/or iii) mono-carbonic acid-mono-ols of PAG (all mentioned hydroxy-groups and carboxyl groups of the compounds i), ii) and iii) mentioned before being the end-groups of PAG), wherein when only i) and iii) are present at least two internal ester groups in the PAG-ester polymer are present, such polymer backbone preferably having a weight average molecular weight Mw of 500 to 50 000 g/mol and a polydispersity PD of 2 to 6, comprising 10 to 560 ether groups and 2 to 51 ester groups, which are interconnected with alkylene groups, and wherein further low molecular weight di-carbonic acids may be contained in addition to the compounds iii).

The polymer backbone (A) may also be described as polyalkylene oxide ester polymer (“PAG-ester” polymer) comprising as building blocks i) diols of polyalkylene oxides (“PAG”), ii) di-carbonic acids of PAG and/or iii) mono-carbonic acid-mono-ols of PAG (all mentioned hydroxy-groups and carboxyl groups of the compounds i), ii) and iii) mentioned before being the end-groups of PAG), wherein when only i) and iii) are present at least two internal ester groups in the PAG-ester polymer are present, wherein further low molecularweight di-carbonic acids may be contained in addition to the compounds ii).

The polymeric sidechains (B) attached on the polymer backbone (A) are obtainable by polymerization of at least one monomer being selected from i) vinyl ester monomer (B1), and further monomer(s) (B2).

As the graft polymers of the invention based on such PAG-ester-polymer show a significantly better biodegradation than conventional graft polymers based on conventional polyalkylene oxide polymers. The present invention further relates to a process for obtaining such a graft polymer, the process is preferably carried out by free-radical polymerization.

Furthermore, the present invention relates to the use of such a graft polymer within, for example, fabric and home care products, and formulations of aroma chemicals.

This invention also relates to fabric and home care products as such and formulations of at least one aroma chemical as such, containing such a graft polymer.

### BACKGROUND

Polyalkylene oxides are important polymers with a wide range of applications. They are, inter alia, used as solvents, consistency enhancer, emulsifier, dispersants, protective colloids, plasticizers, release agents as well as ingredients or raw materials in the production of adhesives and diverse polymers such as graft polymers. Besides various technical applications, they are, for example, also used in a variety of consumer products such as in cosmetics or washing and cleaning agents.

They also have been extensively used as basis to produce graft polymers as further exemplified below. However, a certain amount of such consumer products is rinsed away after their use and may, if not biodegradated or otherwise removed in the sewage treatment plant, end up as microplastics in the river or sea. It was recognized in the course of the invention that the biodegradability of polyalkylene oxides decreases in the range from a few hundred g/mol molecular weight up to a few thousand g/mol molecular weight. However, the polymers described by the current Invention are preferably produced by radical graft polymerization and provide enhanced biodegradation properties compared to the state-of-the-art.

Various countries have already introduced initiatives to ban microplastics especially in cosmetic products. Beyond this ban of insoluble microplastic there is an intense dialog on future requirements for soluble polymers used in consumer products. It is therefore highly desirable to identify better biodegradable ingredients for such applications. Even radically produced graft polymers with a polyethylene glycol backbone show only limited biodegradation in wastewater if the polyethylene glycol backbone is within the molecular weight range mentioned above, and particularly if the molecular weight is above a few thousands g/mol.

Whereas low molecular weight polyethylene oxide with M_{w} of 600 g/mol is easily biodegradable, polyethylene oxide with m_{w} of 6000 g/mol is only poorly biodegradable. BASF's safety data sheet for Pluriol® E 600, revised version 2.0, dated 05. January 2021, affirms for polyethylene glycol with M_{w}=600 g/mol a DOC value (dissolved organic carbon) measured according to OECD 301A of >70%. In contrast to that, the biodegradability of polyethylene glycol with M_{w}=6000 g/mol is mentioned in BASF's safety data sheet for Pluriol® E 6000 Pellet, revised version 2.0, dated 10. August 2018, to be only poor, showing only 10-20% CO₂ formation relative to the theoretical value (60 d) according to OECD 301B.

The classical polyalkylene oxides contain polymer chains of oxyalkylene groups with OH groups at both ends. However, there are also known in the state of the art polyalkylene oxides with functionalized end groups, which show specific properties and allow specific application.

### OBJECT OF INVENTION

It was recognized that the graft polymers based on such conventional polyalkylene oxides show a surprisingly low biodegradation, which is often very much lower than the expected biodegradation percentage which is calculated on the biodegradation of the pure polyalkylene oxides.

The graft polymers being based on such conventional polyalkylene oxides commonly show a decrease in biodegradation compared to the unmodified polyakylene oxides and unmodified polyalkylene glycols, as the degree of modification of polyalkylene oxides (often polyalkylene oxides with two hydroxy-end groups are employed, thus such polyakylene oxides with hydroxy-groups being named commonly “polyalkylene glycols”) with polymerizable monomers by radical grafting onto such backbones increases (i.e. the number of side chains on the backbone increases). This is sometimes attributed to the blocking of the biodegradation mechanism, as it seems that the polyalkylene oxides/glycols are degraded starting from their respective end group then following the polymer chain along. Thus, any additional branching on a carbon-atom of the backbone—which occurs when a polymeric side chain is grafted onto such backbone—impedes and possibly completely stops degradation. As a result, it is suggested that the higher the degree of grafting (i.e. the more side chains are attached to the backbone) the lower is the biodegradation percentage of such graft polymer. Unfortunately it is also commonly observed that with higher degree of branching the performance increases in the desired applications, as only with a higher amount of side chains the chemical structure of the backbone is changed enough that the new graft polymer exerts its specific properties compared to the separated properties of the unmodified backbone in simple mixture with the (unattached/ungrafted) homopolymer which would make up the side chain of the graft polymer.

Hence, the difficulty of combining the conflicting properties of a suitable graft polymer with superior application performance with the biodegradation percentage of the unmodified backbone (i.e. an unmodified polyalkylene oxide/glycol) has not been met up to date.

Hence, there was a need to improve the biodegradation of such graft polymers based on polyalkylene oxides by improving the biodegradability of the graft base and keeping the general structure of the graft polymer and thus maintaining the application performance or even improve it.

Thus, the present invention concerns the improvement of the biodegradability of the graft base and the provision of graft polymers based on such improved graft bases such that the graft polymers themselves show an improved biodegradation over similar graft polymers based on “standard” polyalkylene oxide-polymers but with comparable or even improved performance in the various target applications.

### Prior Art on Polyalkylene Oxide-Esters

CN 110498915 A discloses the preparation of omega hydroxy alpha carboxy polyethylene oxide, in which an ester group functionalized hydroxy compound, such as methyl 2,2-dimethyl-3-hydroxypropionate, is polymerized with ethylene oxide to an omega hydroxy polyethylene oxide alpha ester intermediate product, which is then hydrolyzed to the respective omega hydroxy alpha carboxy polyethylene oxide. The COOH end group is mentioned to serve as a site for reacting with other molecules to form modified polyethylene oxides, e.g. for their use in biological or medical fields.

U.S. Pat. No. 2,585,448 describes polyethylene oxides in which one or both of the OH end groups are esterified with an aromatic or aliphatic carboxylic acid. The mono- and di-esters are mentioned to be useful as plasticizers.

Other documents relate to cyclic polyether ester, which are usually called oxo crown ethers. Oxo crown ethers are cyclic polyalkylene oxides with at least one ester group within the cycle.

JP 55-143981 discloses the preparation of cyclic polyether ester, which are usually called oxo crown ethers. The mentioned oxo crown ethers are cyclic esters with 2 to 9 ether groups and 1 to 2 ester groups. They are synthesized in a multistep synthesis starting with a polyethylene oxide, converting it with sodium metal to the mono sodium salt of the polyethylene oxide, adding sodium bromoacetate under elimination of sodium bromide, further adding p-toluene sulfonyl chloride (also called tosyl chloride) as leaving group to the obtained carboxylate group, and intramolecularly cyclizing the ω-hydroxy-α-tosyl ester in the presence of a templating metal ion under elimination of the tosyl group to the respective oxo crown ether. Oxo crown ethers are mentioned to be mainly used as complexing agents for alkali and alkaline earth metal cations, e.g. in organic synthesis, separation, analysis, biochemistry and pharmaceuticals.

Y. Nakatsuji et al., Synthesis (1981) 42-44 also describe the preparation of oxo crown ethers with 3 to 5 ether groups and one ester group. Polyethylene oxide is reacted with sodium metal and bromoacetic acid obtaining a polyethylene oxide with a terminal methane carboxylate group, which is then esterified with methanol. The obtained ω-hydroxy-α-methyl ester is then either directly cyclized by an intramolecular transesterification to the respective oxo crown ether, or saponified to a polyethylene oxide having a terminal carboxylic acid group and a terminal OH-group, and then intramolecularly cyclized by dehydration.

L. van der Mee et al., J. Polymer Sci. Part A, Polymer Chem. 44(7) (2006) 2166-2176 disclose the preparation of 2-oxa-12-crown-4-ether by conversion of triethylene glycol with t-butylbromoaceate under elimination of sodium bromide, and cyclization of the obtained t-butyl ester in the presence of cobalt dichloride. Furthermore, they disclose the ring-opening polymerization of the obtained 2-oxa-12-crown-4-ether and the copolymerization of 2-oxa-12-crown-4-ether with ω-pentadecanolactone in the presence of Novozym 435 as a catalyst and benzyl alcohol to linear polymers containing either

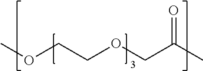

units or mixtures of

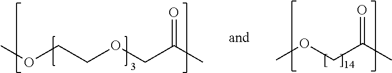

units. Oxo crown ethers are mentioned to be highly interesting monomers for the synthesis of hydrophilic polyesters.

Beside polyalkylene oxides with functionalized end groups and oxo crown ethers, also linear polyalkylene oxides with functionalized groups within the oxyalkylene chain are known in the state of the art.

US 2011/0,207,634 discloses the preparation of polyalkylene oxides with carboxylate end groups, in which the polyalkylene oxide chain may contain exactly one ester group. The polyalkylene oxides with carboxylate end groups and one ester group in the polymer chain are prepared by reacting the corresponding polyalkylene oxides starting material having OH end groups with a base in the presence of a transition metal catalyst under elimination of hydrogen. Ether carboxylates are mentioned as useful mild anionic surfactants.

WO 2001/012,203 relates to a new class of polymers for surgical use which are useful as a sterile adhesion prevention barrier between the tissues of the animal, and which are formed from a polyoxaester having a first repeating unit

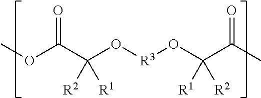

in which R¹ and R² are independently hydrogen or a C₁₋₈ alkyl group, and R³ is a C₂₋₁₂ alkylene group or an oxyalkylene group with up to 2000 repeating units, and having a second repeating unit being either an oxyalkylene group with up to 2000 repeating units or a bivalent unit

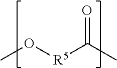

in which R⁵ is a specific alkylene group with up to 17 carbon atoms, a specific oxyalkylene group with three carbon atoms and one oxygen atom, a specific keto unit with 3-7 CH₂ groups and one keto group, or a specific alkylester group with 2-6 CH₂ groups and one —O—CO— group.

U.S. Pat. Nos. 6,147,168, 6,224,894, EP 0,771,832 and EP 0,771,849 disclose further polymers for surgical use which contain the repeating units as specified in WO 2001/012,203 and an additional third repeating unit which is inter alia mentioned to be a bivalent unit

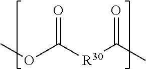

in which R³⁰ is a bivalent alkylene, arylene or arylalkylene group, or a bivalent unit

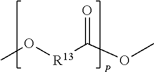

in which R¹³ is a specific alyklene group with up to 17 carbon atoms, a specific oxyalkylene group with three carbon atoms and one oxygen atom, a specific keto unit with 3-7 CH₂ groups and one keto group, or a specific alkylester group with 2-6 CH₂ groups and one —O—CO— group, and P is an integer ensuring that the number average molecular weight of the polymer is less than 1,000,000.

The cited documents relating to linear polyalkylene oxides with functionalized groups within the oxyalkylene chain mention specific applications for these classes of functionalized polyalkylene oxides, such as its use as a mild anionic surfactants or for the production of surgical devices, but are silent on environmental issues and particularly on the biodegradability of such polymers. Furthermore, their synthesis requires at least two isolated components such as dicarboxylic acids and diols which have to be produced beforehand, isolated and purified, which causes a complex production.

### Prior Art on Graft Polymers on Polyalkylene Oxides

WO 2007/138053 discloses amphiphilic graft polymers based on water-soluble polyalkylene oxides (A) as a graft base and side chains formed by polymerization of a vinyl ester component (B), said polymers having an average of <one graft site per 50 alkylene oxide units and mean molar masses M of from 3 000 to 100 000. However, WO 2007/138053 does not describe any backbone material based on block copolymers. Furthermore, WO 2007/138053 does not contain any disclosure in respect of the biodegradability (also named “biodegradation”) of the respective graft polymers disclosed therein.

Y. Zhang et al. J. Coll. Inter. Sci 2005, 285, 80, relates to the synthesis and characterization of specific grafted polymers based on a Pluronic™-type backbone. Pluronic poly(ethylene oxide)-b-poly(propylene oxide)-b-poly(ethylene oxide) (PEO-PPO-PEO) block copolymers are grafted with poly(vinyl pyrrolidone) by free radical polymerization of vinyl pyrrolidone with simultaneous chain transfer to the Pluronic in dioxane. However, Y. Zhang does not disclose that polymeric sidechains of the respective graft polymer are based on vinyl ester monomers. Furthermore, Y. Zhang does not have any disclosure in respect of the biodegradability of the graft polymers disclosed therein. Y. Zhang also does not contain any disclosure about the use of such graft polymer within fabric and home care products.

WO 03/042262 relates to graft polymers comprising (A) a polymer graft skeleton with no mono-ethylenic unsaturated units and (B) polymer sidechains formed from co-polymers of two different mono-ethylenic unsaturated monomers (B1) and (B2), each comprising a nitrogen-containing heterocycle, whereby the proportion of the sidechains (B) amounts to 35 to 55 wt.-% of the total polymer. However, the graft polymers according to WO 03/042262 are not based on vinyl ester monomers within the respective polymer sidechains grafted onto the backbone. Beyond that, WO 03/042262 does not have any disclosure in connection with the biodegradability of the graft polymers disclosed therein.

U.S. Pat. No. 5,318,719 relates to a novel class of biodegradable water-soluble graft copolymers having building, anti-filming, dispersing and threshold crystal inhibiting properties comprising (a) an acid functional monomer and optionally (b) other water-soluble, monoethylenically unsaturated monomers copolymerizable with (a) grafted to a biodegradable substrate comprising polyalkylene oxides and/or polyalkoxylated materials. However, U.S. Pat. No. 5,318,719 does not disclose the use of a block copolymer backbone within the respective graft polymers. Furthermore, the respective sidechain of said graft polymers mandatorily comprises a high amount of acid-functional monomers such as acrylic acid or methacrylic acid. Such type of acid monomers are not useful within the context of the present invention.

Further graft-polymers on polyethylene glycols and polyalkylene glycols are also know from WO00-18375, which employs PEGs which are modified by radical polymerization using vinyl acetate but also claims the use of further monomers such as vinylpyrrolidone, vinylimidazole, vinylcaprolactam and (meth)acrylic acid. In preferred embodiments and also exemplified are PEGs grafted with vinyl acetate which are then hydrolyzed to obtain a “polyvinylalcohol-modified” PEG, with the main use being as a pharmaceutical coating, pharmaceutical binder polymer or film-forming for dosage forms.

The use of such graft polymers similar to those of WO00-18375 but made up from polyalkylene glycols as backbone and vinylpyrrolidone and vinyl acetate as grafted monomers (with no hydrolyzation of the vinyl acetate after polymerization) in detergents are known from US 2019-0390142 A1.

U.S. Pat. No. 6,867,262B1 discloses graft polymers of at least a vinyllactams, preferably vinylcaprolactam, on polyalkylene glycol, a polyether or a polymer having at least one heteroatom in the main chain, optionally also including a vinyl ester as grafted monomer, for use in the inhibition of gas hydrate formations within pipelines in the oil fields.

WO2007051742A1 discloses a process for preparing graft polymers of the type polyethylene glycol grafted with vinyllactams and smaller amounts of vinyl acetate for various uses, as potentially biodegradable gas hydrate inhibitors within oil field applications, and as detergent additive. Biodegradation was said to be achieved but no values are disclosed.

From WO91/19778 it is known to use graft polymers in detergent, with the graft polymers being obtained by grafting of monoethylenically carboxylic acids as grafted monomers onto backbones such as alkylene glycols, poly alkylene glycols, polytetrahydrofurane, glycerine, polyglycerine, or reaction products of the before mentioned compounds with polyvalent carboxylic acids or polyvalent isocyanates.

WO2007138054A1 discloses graft polymers of vinyl acetate on PEG for use in detergents.

Detergent compositions in general are well known in the art and can be formulated in a number of different ways to address a number of different problems. One problem which arises during the washing process of laundry is that redeposition of soil typically occurs which leads to a general greying of fabrics which is sought to be avoided, as e.g. described in EP 3 266 858 A1.

Graft polymers based on conventional polyalkylene oxides were also used in e.g. EP2788467 for automated dish wash application to e.g. improve the drying of hard surfaces; hand dish wash formulations have been likewise formulated with such graft polymers.

### OBJECT

It was an object of the present invention to find a new class of compounds which are able to substitute polyalkylene oxides, particularly polyethylene oxides, polypropylene oxides, poly-1,2-butylene oxides and polytetrahydrofuran, in their typical applications such as for the preparation of graft polymers for its use in homecare and laundering applications, generally in cleaning applications, in agrochemical formulations and other typical applications where graft polymers on conventional polyalkylene oxide polymers have been tested or postulated for using them, where the graft polymers have the same or at least very similar application properties than the products based on conventional polyalkylene oxides, but a better biodegradability.

The parallel-filed EP-application number EP21180239.2 describes the preparation of polyalkylene oxide ester polymers from polyalkylene oxides by selective oxidation and following esterification; those polyalkylene oxide ester polymers serve as graft base for the graft polymers of this present invention. It was also the object to produce graft polymers based on such new polyalkylene oxide ester polymers, with the graft polymer preferably having an improved biodegradability compared to graft polymers based on known polyalkylene oxide-type polymers.

It was an object of this present invention to provide new graft polymers based on these new polyalkylene oxide ester polymers by grafting a polymeric side chain, such as a polymer obtained by polymerization of a vinyl ester monomer and optionally other vinyl monomers, onto the polyalkylene oxide ester polymer.

Moreover, it was also an object of the present invention to indicate the usefulness of the new compounds for various applications, especially in the field of detergent applications.

Furthermore, these novel graft polymers should have beneficial properties in respect of biodegradability and preferably also their washing behavior, their dispersing properties, and/or stabilizing properties, when being employed within compositions such as cleaning compositions, fabric and home care compositions, aroma chemical formulations, pigment dispersions and the like.

A typically desired property is the anti-greying property of such graft polymer when applied in liquid and solid laundry formulations, to reduce the greying of a washed fabric.

It is known to produce the herein employed building blocks “di-carbonic acids of PAG”, as referenced elsewhere within this disclosure.

However, it is not known to produce mono-ol mono-carbonic acids of PAG, i.e. polyalkylene oxide polymers having a hydroxy-group at one end and a carbonic acid group at the other end. Such process and such compounds are the subject of the other already mentioned co-filed patent application with number EP21180239.2, such structures and processes to produce are incorporated herein in its entirety by reference, as this present application makes use of such new compounds.

PAG-Ester polymer backbones as used herein as graft bases as such are not yet known to a person skilled in the art, but the process to produce such products and the products as such are also the subject of the already mentioned co-filed patent application with number the other already mentioned co-filed patent application with number EP21180239.2, such structures and the processes to produce are incorporated herein in its entirety by reference, as this present application makes use of such new compounds.

As used herein, the articles “a” and “an” when used in a claim or an embodiment, are understood to mean one or more of what is claimed or described. As used herein, the terms “include(s)” and “including” are meant to be non-limiting, and thus encompass more than the specific item mentioned after those words.

The compositions of the present disclosure can “comprise” (i.e. contain other ingredients), “consist essentially of” (comprise mainly or almost only the mentioned ingredients and other ingredients in only very minor amounts, mainly only as impurities), or “consist of” (i.e. contain only the mentioned ingredients and in addition may contain only impurities not avoidable in an technical environment, preferably only the ingredients) the components of the present disclosure.

Similarly, the terms “substantially free of . . . ” or “substantially free from . . . ” or “(containing/comprising) essentially no . . . ” may be used herein; this means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or even less than 0.1%, or even more less than 0.01%, or even 0%, by weight of the composition.

The term “about” as used herein encompasses the exact number “X” mentioned as e.g. “about X %” etc., and small variations of X, including from minus 5 to plus 5% deviation from X (with X for this calculation set to 100%), preferably from minus 2 to plus 2%, more preferably from minus 1 to plus 1%, even more preferably from minus 0.5 to plus 0.5% and smaller variations. Of course if the value X given itself is already “100%” (such as for purity etc.) then the term “about” clearly can and thus does only mean deviations thereof which are smaller than “100”.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (° C.) unless otherwise indicated. Unless otherwise specified, all measurements herein are conducted at 20° C. and under the atmospheric pressure. In all embodiments of the present disclosure, all percentages are by weight of the total composition, unless specifically stated otherwise. All ratios are weight ratios, unless specifically stated otherwise.

### Solution

The object of this present invention is achieved by a graft polymer comprising
(A) a polymer backbone as a graft base, wherein said polymer backbone (A) is obtainable by condensation comprising
   i) diols of poly alkylene oxides (PAG),
   ii) di-carboxylic acids of PAG, and/or
   iii) mono-carbonic acid mono-ols of PAG
   wherein either at least a compound selected from iii) is present, or—when only i) and ii) are present—at least two internal ester-groups are present, and
   with the PAG being obtained by polymerization of at least one monomer being selected from 1,2-alkylene oxides such as ethylene oxide, 1,2-propylene oxide, 1,2-butylene oxide, 2,3-butylene oxide, 1,2-pentene oxide or 2,3-pentene oxide; from 1,4-diols or their cyclic or oligomeric analogs, or the PAG being based on polymeric ethers of such 1,4-diols; from 1,6-diols or their cyclic or oligomeric analogs, or the PAG being based on polymeric ethers of such 1,6-diols; or any of their mixtures in any ratio, either as blocks of certain polymeric units, or as statistical polymeric structures, or a polymers comprising one or more homo-blocks of a certain monomer and one or more statistical blocks comprising more than one monomer, and any combination thereof such as polymers having several different blocks of different monomers, or blocks of two different monomers, blocks of statistical mixtures of two or more monomers etc.,
   and optionally other non-polymeric di-carboxylic acid compounds which may be present in addition to compound ii), and
(B) polymeric sidechains grafted onto the polymer backbone, wherein said polymeric sidechains (B) are obtainable by polymerization of at least one monomer being selected from i) vinyl ester monomer (B1) and ii) optionally further monomers (B2).

In an alternative embodiment, the graft polymer based on PAG-ester is a graft polymer comprising
(A) a polymer backbone as a graft base, wherein said polymer backbone (A) is a polyalkylene oxide ester polymer with a weight average molecular weight M_{w} of 500 to 50 000 g/mol and a polydispersity PD of 2 to 6, comprising 10 to 560 ether groups and 2 to 51 ester groups, which are interconnected with alkylene groups, which contains 1 to 51 structural elements of the general formula (I)

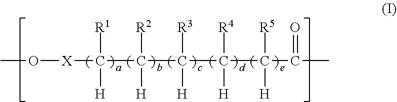

in which
   the —O— unit at the left side is bound to a —CO— unit of an adjacent unit of the polymer, forming an ester unit,
   the —CO— unit at the right side is bound to a —O— unit of an adjacent unit of the polymer, forming a further ester unit,
   R¹, R², R³, R⁴, R⁵ represent independent of each other a hydrogen atom or a C₁₋₁₂ alkyl group,
   a, b, c, d, e represent independent of each other an integer of 0 or 1, whereas the sum of a to e is 1 to 5, and
   X represents a polyalkylene oxide unit with 4 to 100 alkylene oxide units, whereby the alkylene oxide units contain independent of each other 2 to 6 carbon atoms in the direct chain between two —O— units, whereby each of the carbon atoms in the direct chain between two —O— units contain independent of each other either two hydrogen atoms, or one hydrogen atom and one C₁₋₁₂ alkyl group, and
(B) polymeric sidechains grafted onto the polymer backbone, wherein said polymeric sidechains (B) are obtainable by polymerization of at least one monomer being selected from i) vinyl ester monomer (B1) and ii) optionally further monomers (B2).

The graft polymers according to the present invention may be used, for example, within cleaning compositions and/or fabric and home care products and/or agrochemical formulations. They lead to an at least comparable and preferably even improved anti redeposition and cleaning performance within such compositions or products, for example in respect of redeposition of soils and removing of stains, avoiding or reducing re-soiling or greying or depositioning of solids, dispersion of actives in agrochemical formulations, inhibiting crystal growth etc. compared to corresponding polymers or graft polymers according to the prior art. They may be also advantageously being used—partly also depending on the monomer(s) B employed for grafting and thus adjusted in their performance to the specific needs of the specific applications; such monomer substitution pattern as possibly also derivable from the prior art of analogous graft polymers based on simple PEGs and polyalkylene glycols—for inhibiting gas hydrate formation, improve pigment dispersion stability, hydrophobisation of surfaces, reduction of growth of microbes on such surfaces, and/or odor control.

Beyond the performance in a certain type of application, the graft polymers according to the present invention lead to an improved biodegradability when being employed within such compositions or products, compared to the previously known graft polymers.

Graft polymers with enhanced biodegradation according to the current invention can be used advantageously in washing and cleaning compositions, where they support the removal of hydrophobic soils from textile or hard surfaces by the surfactants and thus improve the washing and cleaning performances of the formulations. Moreover, they bring about better dispersion of the removed soil in the washing or cleaning liquor and prevent its redeposition onto the surfaces of the washed or cleaned materials.

In another embodiment they can be employed in agrochemical formulations containing agrochemical active ingredients; in such agrochemical formulations the graft polymers serve to disperse, avoid sedimentation, emulsify and/or stabilize such formulations, by e-g- acting on the agrochemical active such as avoiding or reducing the crystal growth of (semi-)crystalline actives and/or the formulation ingredients as such or in the formulation as such.

The terms “polymer (backbone)”, “graft base” and “PAG-ester” and “PAG-ester polymer” are used herein interchangingly all meaning the polyalkylene oxide ester as disclosed in the parallel-filed EP-application number EP21180239.2 and detailed herein as well, which serve as graft base for the graft polymers of this present invention.

### PAG-Ester Properties

The polyalkylene oxide ester (PAG-ester) used as polymer backbone of the invention is characterized by its weight average molecular weight M_{w}, its polydispersity PD, its number of ether groups, its number of ester groups, and the existence of at least one structural element (I).

The weight average molecular weight M_{w} of the polyalkylene oxide ester polymer used as polymer backbone of the invention is 500 to 50 000 g/mol. M_{w} includes the mass of individual chains, which contributes to the overall molecular weight of the polymer and considers that bigger molecules contain more mass than smaller molecules. It is determined by size exclusion chromatography (SEC) in a liquid-solid phase and detection by differential light refraction against a reference cell, whereas the unit is calibrated with a polymer of known molecular weight. M_{w} is then calculated by computational methods based on the course of the curve of the chromatogram. Such a method is well known in the art. The weight average molecular weight M_{w} is preferably ≥750 g/mol, more preferably ≥1000 g/mol, particularly preferably ≥2000 g/mol, very particularly preferably ≥3000 g/mol and most preferably ≥4000 g/mol, and preferably ≤45 000 g/mol, more preferably ≤40 000 g/mol, particularly preferably ≤35 000 g/mol, very particularly preferably ≤25 000 g/mol and most preferably ≤15 000 g/mol.

Since the weight average molecular weight M_{w} is only a mean value of the molecular weight without information on the distribution of the molar weights of the individual molecules, the polyalkylene oxide ester polymer used as polymer backbone of the invention is further specified by the polydispersity PD. The polydispersity PD is defined as M_{w}/Mₙ, whereas Mₙ is the number average molecular weight specifying the ordinary arithmetic mean or average of the molecular weights of the individual molecules. The polyalkylene oxide ester polymer of the invention has a polydispersity PD of 2 to 6, preferably ≥2.5 and more preferably ≥3, and preferably ≤5.

The number average molecular weight Mₙ is preferably 250 to 20 000 g/mol, more preferably ≥500 g/mol and particularly preferably ≥1000 g/mol, and more preferably ≤15 000 g/mol and particularly preferably ≤10 000 g/mol.

Biodegradability is the ability of organic substances to be broken down into simpler substances through the action of enzymes from microorganisms. The degradation process consumes oxygen and produces carbon dioxide. Both can be measured by certain tests. Worldwide accepted tests have been published in the OECD 301 guideline for testing of chemicals. Depending on the specific test method, dissolved organic carbon (DOC), carbon dioxide evolution or the oxygen consumption is measured over time during the degradation under standardized conditions.

Based on OECD measurements, the polyalkylene oxide ester polymer as used in this invention as polymer backbone A of the invention can usually be biodegraded by 70 to 90% within one month, even with a weight average molecular weight Mw of 20 000 g/mol, whereas conventional polyalkylene oxide polymers only reach values of less than 20% or even less than 10%.

### Detailed Structure of PAG-Ester

The polymer backbone (A) of the inventive graft polymer is a polyalkylene oxide ester polymer (“PAG-ester” polymer) comprising as building blocks i) diols of polyalkylene oxides (“PAG”), ii) di-carbonic acids of PAG and/or iii) mono-carbonic acid-mono-ols of PAG (all mentioned hydroxy-groups and carboxyl groups of the compounds i), ii) and iii) mentioned before being the end-groups of PAG), wherein when only i) and iii) are present at least two internal ester groups in the PAG-ester polymer are present, such polymer backbone preferably having a weight average molecular weight Mw of 500 to 50 000 g/mol and a polydispersity PD of 2 to 6, comprising 10 to 560 ether groups and 2 to 51 ester groups, which are interconnected with alkylene groups, and wherein further low molecular weight di-carbonic acids may be contained in addition to the compounds iii).

The polymer backbone (A) may also be described as polyalkylene oxide ester polymer (“PAG-ester” polymer) comprising as building blocks i) diols of polyalkylene oxides (“PAG”), ii) di-carbonic acids of PAG and/or iii) mono-carbonic acid-mono-ols of PAG (all mentioned hydroxy-groups and carboxyl groups of the compounds i), ii) and iii) mentioned before being the end-groups of PAG), wherein when only i) and iii) are present at least two internal ester groups in the PAG-ester polymer are present, wherein further low molecular weight di-carbonic acids may be contained in addition to the compounds ii).

The PAG-ester comprises at least one, preferably at least two, and more preferably and least three different polymer subunits linked by covalent ester-bonds, such structure formed by any of the following three options:

In one embodiment, the two compounds forming the ester-bond by condensation are
i) mono-ol mono-carbonic acid of poly alkylene oxide (PAG) with
ii) itself, respectively;
   in another embodiment, the two compounds forming the ester-bond by condensation are
i) di-ol of poly alkylene oxide (PAG) with
ii) PAG containing two carbonic acids as end-group, i.e. a carbonic acid-group at both ends of the PEG (or PAG, respectively);
   in a third embodiment, a mixture of both embodiments before is employed, i.e. a condensation of
i) mono-ol mono-carbonic acid of poly alkylene oxide (PAG) with
ii) di-ol of poly alkylene oxide (PAG) and
iii) PAG containing two carbonic acids as end-group, i.e. a carbonic acid-group at both ends of the PEG (or PAG, respectively).

The third embodiment can be exemplified by using mixtures containing all three required compounds, i.e. mono-ol mono-carbonic acid of polyalkylene oxide (PAG), di-ol of poly alkylene oxide (PAG) and PAG-di carbonic acid. Such mixtures can be prepared e.g in one step by partial oxidation of PAG-Diols by incomplete oxidation, i.e. the oxidation is stopped after some hours to obtain directly a mixture containing those three components.

On the other hand, it is of course also possible to obtain the PAG-ester from mixtures which are specifically composed by adding the required starting materials.

Preferably, the synthetic route of the third embodiment is the preferred option for obtaining the PAG-ester for use as backbone to obtain the graft polymers of the present invention.

Further, it is of course possible and encompassed by this present invention, that more than one specific PAG-polymer is employed in the reactions of the previous three embodiments: Hence it is also possible in all three embodiments defined before that for example PEG and another PAG are employed as the “PAG”-part, such that for example diols of PEG are combined with di-acids of a PAG other than PEG, or monol-mono-acids of PEG with monool-mono-acids of PAG other than PEG, or mixtures of monool-mono-acid of PEG with monol-mono-acid of PAG other than PEG and di-acid of PAG other than PEG and/or PEG etc. All thinkable combinations are of course possible and are meant to be encompassed by the concept of the present invention, thus enabling to tune the hydrophilicity/hydrophobicity of the resulting polymer backbone and thus providing a further variable to tune the properties of the desired graft polymers using such PAG-ester polymer backbones.

“PAG” as used herein are polyakylene oxide-polymers of any type as defined herein; however, only when “PAG” is used in direct comparison to “PEG” (i.e. a PAG prepared solely from ethylene oxide, which is commonly known as “PEG”, denoting pure ethylene oxide-homo polymers) only than “PAG” is intended to mean another PAG not being PEG.

Such PAGs not being PEG could be derived from propylene oxide, butylene oxide or higher alkylene oxides up to C10, or mixtures of two or more of C2- to C10-alkylene oxides, such as for example mixtures of ethylene oxide and propylene oxide.

PAG could consists of just one alkylene oxide-monomer-type, or of two, three, four or more different alkylene oxide-monomer-types, and thus could be for example of block copolymers of two or more alkylene oxides, e.g. polymers of ethylene oxide and propylene oxide, either as block polymers or random polymers, or polymers comprising mixed structures of block units (with each block being a homo-block or a random block itself) and statistical/random parts.

A “two-block” PAG has two distinct blocks (polymer subunits), whereas “triblock” PAG have, by consequence, three distinct blocks (polymer subunits) and so on. The number of individual blocks within such block copolymers is not limited, by consequence, a “n-block copolymer” comprises n distinct blocks (polymer subunits). Within the individual blocks (polymer subunits) the size/length of such a block may vary. The smallest length/size of a block is based on two individual monomers (as a minimum).

In case the PAG employed for the oxidation to produce the PAG-Ester polymers to be used as graft base is made up from more than one different monomer (single monomeric unit), then the polymer chain of PAG may be in the form of blocks with a block of a first single monomeric unit attached to a block of a second single monomeric unit which is different to the first monomeric unit; such polymeric chains may contain more than two blocks such as three, four, five or more blocks, all such blocks and block structures being obtainable by standard means. Instead of blocks of a single monomeric unit each block may also be made up from more than one monomeric unit with the monomers being statistically distributed within one such specific block; of course it is also possible to obtain by standard means combinations of blocks made up from single monomeric units with blocks being made up by more than one single monomeric unit; all such thinkable combinations of the before mentioned possibilities are in principle possible and obtainable by standard means; preferred structures are—due to their ease of obtaining them—a PAG being made up from a single monomeric unit, or prepared by a statistical mixture of more than one monomeric unit, or prepared as two or three blocks or more—with up to three blocks being preferred and only up to two blocks being even more preferred—preferably with the two or more blocks being made up each from just one single monomeric unit per block.

Although the intention during producing for example a PAG being made up of a two-block-structure with two different single monomeric units and each block intended to be a homo-polymeric block of a different single monomeric unit, such blocks may nevertheless contain “dirty structures”: it is understood by a person of skill that due to residing unreacted monomers used for polymerizing the first block there will be no sharp border between the two blocks, but the beginning of the second block my contain a “dirty” structure, i.e. may contain a few of the monomeric units used for the first block which did not react during the time allowed for such first block-polymerisation, but reacting only when the second monomeric unit to be polymerized has been added to the reaction zone. Such dirty structures are obtained when the reaction of the first monomeric unit leading to the first block of the shell is not stopped and the reaction vessel is not emptied from unreacted first monomeric unit, but the reaction is continued after (only almost) “completion” of the polymerization of the first block by adding the second monomeric unit and continue polymerization without a break and without a cleaning in between. For commercial reasons, it is preferred to continue polymerization without break/cleaning, and thus preferable structures will contain such dirty structures. In another embodiment it is preferred that no dirty structures are contained.

Overall, it is important that the PAG employed for the oxidation has HO—CH₂— end-groups or, as only those are oxidizable to carboxylic groups.

A preferred embodiment of the present invention relates to a graft polymer comprising
(A) a PAG-ester polymer backbone as a graft base, wherein said PAG-ester polymer backbone (A) is obtainable by condensation of
   i) diols of poly alkylene oxide (PAG-Diol or PAG-DO) with
   ii) PAG containing di-carbonic acids as end-group (PAG-DC);
   the PAG can individually be obtained by polymerization of at least one monomer selected from the group of ethylene oxide, 1,2-propylene oxide, 1,2-butylene oxide, 2,3-butylene oxide, 1,2-pentene oxide, 2,3-pentene oxide or C5- to C10-alkylene oxides, preferably C2 to C4, more preferably C2 and C3, and most preferably only C2; and
(B) polymeric sidechains grafted onto the PAG-ester polymer backbone, wherein said polymeric sidechains (B) are obtainable by polymerization of at least one monomer being selected from i) vinyl ester monomer (B1) and ii) further monomers (B2).

Another preferred embodiment of the present invention relates to a graft polymer comprising
(A) a PAG-ester polymer backbone as a graft base, wherein said PAG-ester polymer backbone (A) is obtainable by condensation of
   i) mono-ol mono-carbonic acid of poly alkylene oxide (PAG-MC), with
   ii) diols of poly alkylene oxide (PAG-DO) and
   iii) PAG containing di-carbonic acids as end-group (PAG-DC); each PAG can individually be obtained by polymerization of at least one monomer selected from the group of ethylene oxide, 1,2-propylene oxide, 1,2-butylene oxide, 2,3-butylene oxide, 1,2-pentene oxide, 2,3-pentene oxide or C5- to C10-alkylene oxides, preferably C2 to C4, more preferably C2 and C3, and most preferably only C2; and
(B) polymeric sidechains grafted onto the PAG-ester polymer backbone, wherein said polymeric sidechains (B) are obtainable by polymerization of at least one monomer being selected from i) vinyl ester monomer (B1) and ii) further monomers (B2).

Another preferred embodiment of the present invention relates to a graft polymer comprising
(A) a PAG-ester polymer backbone as a graft base, wherein said PAG-ester polymer backbone (A) is obtainable by condensation of
   i) mono-ol mono-carbonic acid of poly alkylene oxide (PAG-MC);
   the PAG can individually be obtained by polymerization of at least one monomer selected from the group of ethylene oxide, 1,2-propylene oxide, 1,2-butylene oxide, 2,3-butylene oxide, 1,2-pentene oxide, 2,3-pentene oxide or C5- to C10-alkylene oxides, preferably C2 to C4, more preferably C2 and C3, and most preferably only C2; and
(B) polymeric sidechains grafted onto the PAG-ester polymer backbone, wherein said polymeric sidechains (B) are obtainable by polymerization of at least one monomer being selected from i) vinyl ester monomer, preferably vinyl acetate, (B1) and ii) further monomers (B2).

For the three preferred embodiments before is also possible and thus encompassed in the present invention to obtain structures of the polymer backbone by choosing among the following starting material: PAG(1)-diols, PAG(2)-di carbonic acids, PAG(3)-mono-ol-mono-carbonic acids wherein PAG(1), PAG(2) and PAG(3) are different poly alkylene oxides; e.g. PAG(1) may be a pure PEG, PAG(2) may be an EO-PO-polymer and PAG(3) may be an EO-butylene oxide-polymer. Of course, each PAG may be chosen individually as being composed by any amount and—if more than one alkylene oxide is chosen for a PAG—of any ratio of C2- to C12-alkylene oxides.

Generally, and specifically for the previously defined three preferred embodiments, the ratio of the polymer backbone (A) versus the polymeric side chains (B) within the graft polymers according to the present invention is not limited to specific values. Any ratio known to a person skilled in the art can be employed. However, it is understood that the graft polymers comprise more than 0.2% by weight of the polymeric sidechains (B) (in relation to the total weight of the graft polymer). Preferably the graft polymers comprise more than 1% by weight of the polymeric sidechains (B) (in relation to the total weight of the graft polymer). More preferably, graft polymers comprise 20 to 95% by weight of the block copolymer backbone (A) and 5 to 80% by weight of the polymeric sidechains (B) (in relation to the total weight of the graft polymer).

Preferably, and specifically for the previously defined three preferred embodiments, the graft polymer comprises 40 to 85% by weight, more preferably 50 to 80% by weight, even more preferably 55 to 75% by weight of the PAG-polymer backbone, preferably the PEG-polymer (A), and preferably 15 to 60% by weight, more preferably 20 to 50% by weight, even more preferably-20 to 50% by weight, even more preferably 25 to 45% by weight of the polymeric sidechains (B) (in relation to the total weight of the graft polymer).

In a preferred embodiment, the PAGs employed based essentially on ethylene oxide, such as PEGs. In a more preferred embodiment, the PAG as employed for preparing the PAG-ester polymer (via the three compounds mono-ol-mono-carbonic acid of PAG, the PAG-di carbonic acid and the PAG-diol) are based on more than 70 weight percent, even more preferably on more than 90 weight percent of ethylene oxide, and most preferably is a homo-poly ethylene oxide, i.e. is “PEG”, such that the PAG-ester polymer employed for preparing the graft polymer of the invention is purely based on PEG only and no other alkylene oxide within PAG.

The graft polymer according to the present invention may have any molecular weight known to a person skilled in the art. However, it is preferred that the graft polymer has a weight average molecular weight M_{w} of from 1 000 to 500 000 g/mol, preferably from 2 000 to 200 000 g/mol, more preferably from 5 000 to 100 000 g/mol, even more preferably from 7 500 to 50 000 g/mol, with the various lower ends of course being possible to combine also with the various upper ends, such as 1000 to 50000 g/mol, 5000 to 50000 g/mol, 2000 to 50000 g/mol etc, all such combination being encompassed by this present invention.

The graft polymers according to the present invention preferably have a low polydispersity of not more than 6. It is preferred that the graft polymer has a polydispersity M_{w}/Mn of <4, preferably <3.5, more preferably <3, and most preferably in the range from 1.2 to 2.5 (with M_{w}=weight average molecular weight and Mₙ=number average molecular weight; with polydispersity being without unit [g/mol/g/mol]). The respective values of M_{w} and/or Mₙ can be determined as described within the experimental section below.

The polymer backbone (A) contained within the graft polymer according to the present invention may either be capped or not capped (uncapped) at the respective end-groups of the backbone. By consequence, within the present invention, it is possible that the polymer backbone (A) is optionally capped at one or both end-groups, preferably the polymer backbone (A) is not capped at both end-groups or, if the polymer backbone (A) is capped, the capping is done preferably by C₁-C₂₅-alkyl groups, which are linked to the backbone chain as ether group or within an ester-group, depending on the actual end-group pf the PAG employed. Such capping may be done using known means, and typically is done before the grafting polymerization is performed.

The PAG-polymers can contain different levels of the hydrophilic ethylene glycol-unit which influences the overall properties of the graft polymer, especially the solubility in water.

Generally, it is observed that higher EO-contents lead to a higher hydrophilicity and thus to a higher solubility in water.

Also, higher EO-contents also lead to higher biodegradation.

Hence, it is preferred in this invention to have medium to high, and more preferably high EO-contents in case a high hydrophilicity is desired.

In another embodiment, the structure of the PAG-ester for use as graft base for the graft polymers of the invention is a polyalkylene oxide ester polymer with a weight average molecular weight Mw of 500 to 50 000 g/mol and a polydispersity PD of 2 to 6, comprising 10 to 560 ether groups and 2 to 51 ester groups, which are interconnected with alkylene groups, which contains 1 to 51 structural elements of the general formula (I)

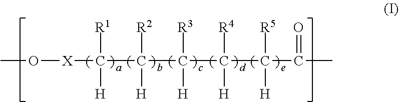

in which
   the —O— unit at the left side is bound to a —CO— unit of an adjacent unit of the polymer, forming an ester unit,
   the —CO— unit at the right side is bound to a —O— unit of an adjacent unit of the polymer, forming a further ester unit,
   R¹, R², R³, R⁴, R⁵ represent independent of each other a hydrogen atom or a C₁₋₁₂ alkyl group,
   a, b, c, d, e represent independent of each other an integer of 0 or 1, whereas the sum of a to e is 1 to 5, and
   X represents a polyalkylene oxide unit with 4 to 100 alkylene oxide units, whereby the alkylene oxide units contain independent of each other 2 to 6 carbon atoms in the direct chain between two —O— units, whereby each of the carbon atoms in the direct chain between two —O— units contain independent of each other either two hydrogen atoms, or one hydrogen atom and one C₁₋₁₂ alkyl group.

The crucial characteristic of the polyalkylene oxide ester polymer used in the invention, which surprisingly enables a high biodegradability, is the existence of ester groups within the polyalkylene oxide polymer chain. Polyalkylene oxide units, which as such are at least fairly biodegradable, and ester groups are linked to each other. Since the polyalkylene oxide units itself alternately contain ether groups and alkylene groups, the polyalkylene oxide ester polymer can also be described as a polymer containing ether groups and ester groups, which are interconnected with alkylene groups. For the sake of good order, it is pointed out that the term “polyalkylene oxide” does not embrace acetal or ketal units in which one carbon atom is interlinked with two ether groups, such as —O-CH2-O—. This is consistent with the common use of the term polyalkylene oxide and known by the person skilled in the art.

In the context of this document, the term “ether group” specifies a —O— unit, which is on both sides bound to carbon atoms which, independent of one other, have an oxidation state of −2, −1 or 0, and which are further bound to hydrogen atoms or other carbon atoms, such as for example −2 for a methyl group, −1 for an unsubstituted alkylene group or 0 for an alpha alkyl substituted alkylene group. In analogy to that, the term “ester group” specifies a —CO— unit which is at one side bound to a carbon atom, which has an oxidation state of −3, −2, −1 or 0, such as for example −3 for a methyl group, −2 for an unsubstituted alkylene group further bound to another carbon atom in the polymer chain, −1 for an alpha alkyl substituted alkylene group further bound to another carbon atom in the polymer chain or for an unsubstituted alkylene group further bound to an —O— group, or 0 for an alpha alkyl substituted alkylene group further bound to an —O— group, and at the other side to a —O— unit which in turn is at the opposite side bound to a carbon atom with an oxidation state of −2, −1 or 0.

Specifically, the polyalkylene oxide ester polymer as used in the invention contains 10 to 560 ether groups and 2 to 51 ester groups, which are interconnected with alkylene groups, wherein it contains 1 to 51 structural elements of the general formula (I)

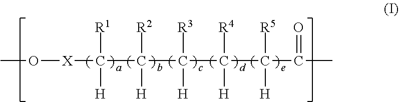

in which
the —O— unit at the left side is bound to a —CO— unit of an adjacent unit of the polymer, forming an ester unit,
the —CO— unit at the right side is bound to a —O— unit of an adjacent unit of the polymer, forming a further ester unit,
R¹, R², R³, R⁴, R⁵ represent independent of each other a hydrogen atom or a C₁₋₁₂ alkyl group,
a, b, c, d, e represent independent of each other an integer of 0 or 1, whereas the sum of a to e is 1 to 5, and
X represents a polyalkylene oxide unit with 4 to 100 alkylene oxide units, whereby the alkylene oxide units contain independent of each other 2 to 6 carbon atoms in the direct chain between two —O— units, whereby each of the carbon atoms in the direct chain between two —O— units contain independent of each other either two hydrogen atoms, or one hydrogen atom and one C₁₋₁₂ alkyl group.

The number of the ether groups specified above as 10 to 560 and the number of the ester groups specified above as 2 to 51 relate to the individual polyalkylene oxide ester polymer molecules. Due to the polydispersity PD, the number of the ether groups and ester groups of the specific polyalkylene oxide ester polymer molecules, which constitute the polyalkylene oxide ester polymer with the weight average molecular weight Mw of 500 to 50 000 g/mol and the polydispersity PD of 2 to 6, show an individual distribution. Consequently, the polyalkylene oxide ester polymer typically contains polyalkylene oxide ester polymer molecules with different numbers of ester groups and ether groups.

Based on the weight average molecular weight M_{w}, the polydispersity PD of the polyalkylene oxide ester polymer and the ratio of the ether groups and ester groups, which can be analytically determined with the knowledge of the person skilled in the art, the average numbers of ether groups and ester groups of the polyalkylene oxide ester polymer can be determined.

The adjacent units of the polymer bound to the —O— unit at the left, and the —CO— unit at the right side of formula (I) contain further alkylene, ether and ester groups, or form together with the mentioned —O— and —CO— units ester groups, respectively, in a number to form a polyalkylene oxide ester polymer, which contains a total number of ether groups and ester groups within the specified range.

The end groups of the polyalkylene oxide ester polymer can principally be any end groups which are suitable for forming the ends of such a polymer. Examples of suitable end groups are —OH, —COOH, primary, secondary or tertiary amine groups, branched or linear alkyl groups, aralkyl groups, aromatic groups, hydroxyalkyl groups, carbonyl groups, carboxyl groups, carboxylic acid ester groups, amide groups, urethan groups, carbamide groups, xanthogenate groups, dithiocarbamate groups or carbamate groups.

However, —OH, —COOH, carboxyl groups, hydroxyalkyl groups and alkyl groups are typically preferred, especially —OH and —COOH.

The radicals R¹, R², R³, R⁴, R⁵ in formula (I) represent independent of each other a hydrogen atom or a C₁₋₁₂ alkyl group. The alkyl groups can be linear or in case of C₃₋₁₂ alkyl be linear or branched. Preferred C₁₋₁₂ alkyl groups are methyl, ethyl, n-propyl, iso-propyl, n-butyl, iso-butyl, tert-butyl, n-pentyl, n-hexyl, n-heptyl, n-octyl, n-nonyl, n-decyl, n-undecyl and n-dodecyl. Preferably, R², R³, R⁴ and R⁵ represent a hydrogen atom, and R¹ a hydrogen atom or a C₁₋₁₂ alkyl group. More preferably, R¹ represents a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularty preferably a hydrogen atom, methyl, ethyl, n-propyl or n-decyl, very particularty preferably a hydrogen atom or methyl, and most preferably a hydrogen atom.

The indices a, b, c, d, e represent independent of each other an integer of 0 or 1, whereas the sum of a to e is 1 to 5. Preferably, a, b, care 1, and d, e are 0. More preferably, a is 1, and b, c, d, e are 0.

A specifically preferred structural element based on formula (I) is the element of the general formula (Ia)

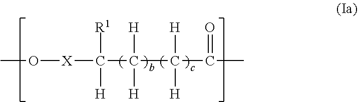

in which
R¹ represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
b, c represent an integer of 0 or 1, whereas the sum of b to c is 0 or 2.

A further specifically preferred structural element based on formula (Ia) is the element of the general formula (Ib)

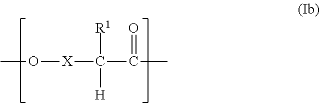

in which
R¹ represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The unit X in formula (I) represents a polyalkylene oxide unit with 4 to 100 alkylene oxide units, whereby the alkylene oxide units contain independent of each other 2 to 6 carbon atoms in the direct chain between two —O— units, which can also be denoted as a C₂₋₆ alkylene unit, whereby each of the carbon atoms in the direct chain between two —O— units contain independent of each other either two hydrogen atoms, or one hydrogen atom and one C₁₋₁₂ alkyl group. The preferred unit X can be expressed by the general formula (Ic)

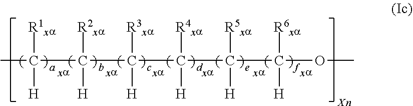

in which
α represents an index from 1 to Xn specifying the running count for each repeating unit,
R¹_{xα}, R²_{xα}, R³_{xα}, R⁴_{xα}, R⁵_{xα}, R⁶_{xα} represent independent of each other, and under consideration that a is a running count for each repeating unit, a hydrogen atom or a C₁₋₁₂ alkyl group,
a_{xα}, b_{xα}, c_{xα}, d_{xα}, e_{xα}, f_{xα} represent independent of each other, and under consideration that α is a running count for each repeating unit, an integer of 0 or 1, whereas the sum of a_{xα} to f_{xα} is 2 to 6, and
Xn represents an integer of 4 to 100.

The small letter x in the radicals, as for example in R¹_{aα}, and in the indices, as for example in a_{xα}, indicate that they refer to unit X. The same is indicated by the capital letter X in the number of the repeating units Xn. Furthermore, the small letter a in the radicals, as for example in R¹_{xα}, and in the indices, as for example in a_{xα}, specify that each of the radicals and indices have their own sub-number, indicating that the radicals and indices may vary from one alkylene oxide unit to the other within a polyalkylene oxide unit X. For example, radical R¹ₓ₁ of the alkylene oxide unit with the running count 1 may be a hydrogen atom, whereas R¹ₓ₂ of the alkylene oxide unit with the running count 2 may be methyl group, and so on. Similarly and also to be understood as an example, the index cₓ₁ of the alkylene oxide unit with the running count 1 may be 0, whereas cₓ₂ of the alkylene oxide unit with the running count 2 may be 1, and so on.

The C₁₋₁₂ alkyl group in the radicals R¹_{xα}, R²_{xα}, R³_{xα}, R⁴_{xα}, R⁵_{xα}, R⁶_{xα} in formula (Ic) can be linear or in case of C₃₋₁₂ alkyl be linear or branched. Preferred C₁₋₁₂ alkyl groups are methyl, ethyl, n-propyl, iso-propyl, n-butyl, iso-butyl, tert-butyl, n-pentyl, n-hexyl, n-heptyl, n-octyl, n-nonyl, n-decyl, n-undecyl and n-dodecyl. Preferably, R²_{xα}, R³_{xα}, R⁴_{xα}, R⁵_{xα} and R⁶_{xα} represent a hydrogen atom, and R¹_{xα} a hydrogen atom or a C₁₋₁₂ alkyl group. More preferably, R¹_{xα} represents a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The indices a_{xα}, b_{xα}, c_{xα}, d_{xα}, e_{xα}, f_{xα} represent independent of each other an integer of 0 or 1, whereas the sum of a_{xα} to f_{xα} is 2 to 6. Preferably, a_{xα}, b_{xα}, c_{xα}, f_{xα} are 1, and d_{xα}, e_{xα} are 0. More preferably, a_{xα}, f_{xα} are 1, and b_{xα}, c_{xα}, d_{xα}, e_{xα} are 0.

A specifically preferred unit X is the unit of the general formula (Id)

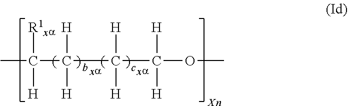

in which
R¹_{xα} represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
b_{xα}, c_{xα} represent an integer of 0 or 1, whereas the sum of b to c is 0 or 2, and
Xn represents an integer of 4 to 100.

A further specifically preferred unit X based on formula (Id) is the unit of the general formula (Ie)

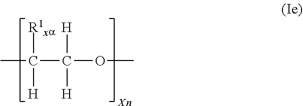

in which
R¹_{xα} represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
Xn represents an integer of 4 to 100.

The number of the repeating units Xn in formula (Ic) is an integer of 4 to 100. It is preferably ≥5, more preferably ≥8 and particularly preferably ≥10, and preferably ≤75 and more preferably ≤50.

It is explicitly emphasized that the alkylene oxide units in unit X can be the same with unit X, or differ from each other, in terms of different radicals R¹_{xα} to R⁶_{xα} and in terms of different indices a_{xα} to f_{xα}. This is already clearly indicated by the index xα in formula (Ic), specifying sub-numbers for each radical, as for example in R¹_{xα}, and for each index, as for example in a_{xα}, based on the running count for each repeating unit.

Moreover, it is explicitly emphasized that in the polyalkylene oxide ester polymer also each structural element (I), if more than one element (I) is present, may be identical to one or more of the others, or may differ from one or more of the others.

The different parts of the structural element (I) such as the radicals, indices and unit X including their general and preferred values have already been described above. The following paragraphs relate to preferred specific combinations of these parts.

Particularly preferred is a polyalkylene oxide ester polymer containing the structural element (I), in which
R¹ represents a hydrogen atom or a methyl group,
R², R³ represent a hydrogen atom,
d, e are 0,
a is 1,
b, c represent an integer of 0 or 1, whereas the sum of b to c is 0 or 2, and
X represents a polyalkylene oxide unit with 4 to 100 alkylene oxide units, whereby the alkylene oxide units are independent of each other and contain 2 or 4 carbon atoms in the direct chain between two ether groups, whereby in each alkylene oxide unit, and independent of each other, one of the carbon atoms in α-position to an —O— unit contain either two hydrogen atoms, or one hydrogen atom and one methyl group, and the other one or three carbon atoms contain two hydrogen atoms each, whereby each —O— unit carries not more than one methyl group carrying carbon atom in α-position.

This particularly relates to a respective polymer in which the structural element (I) is solely formed by C₂-units, solely formed by C₄-units or formed by a mixture thereof. Each C₂- and C₄-unit may either carry only hydrogen atoms, or hydrogen atoms and one methyl group. If a methyl group in a C₂- or C₄-unit is present, it is bound at a carbon atom in α-position to an —O— unit, whereby each —O— unit carries in its α-position not more than one methyl group carrying carbon atom.

Such elements are typically based on ethylene oxide monomers, propylene oxide monomers, tetrahydrofuran monomers or mixtures thereof. In case of propylene oxide based —CHCH₃—CH₂—O— and tetrahydrofuran based —CH₂—CH₂—CH₂—CH₂—O— units within formula (I), it may be advantageous if the structural element (I) contains at the border region of element (I) one or more C₂-based units without methyl groups. This can be easily achieved by firstly polymerizing propylene oxide or tetrahydrofuran and later stop the addition of propylene oxide and tetrahydrofuran, respectively, and supply ethylene oxide to finish the polymerization, leading to C₂-based units at both end sides. The obtained polyalkylene oxides can then be processed as described further down to form the structural unit (I) in the polyalkylene oxide ester polymer. Due to the mentioned production process, ethylene oxide is co-polymerized with propylene oxide and tetrahydrofuran, respectively, causing an irregular structure in the border region in which —CHCH₃—CH₂—O— and —CH₂—CH₂—CH₂—CH₂—O— units, respectively, alternate with —CH₂—CH₂—O— units, so that the transition from —CHCH₃—CH₂—O— and —CH₂—CH₂—CH₂—CH₂—O— units, respectively, to —CH₂—CH₂—O— units might not be sharp. Such effect is well known in the art and the respective alternating structure also often called “dirty structures”.

Another particularly preferred polyalkylene oxide ester polymer contains the structural element (I), in which

R¹ represents a hydrogen atom or a methyl group,
b, c, d, e are 0,
a is 1, and
X represents a polyalkylene oxide unit with 4 to 100 alkylene oxide units, whereby the alkylene oxide units are independent of each other and contain 2 carbon atoms in the direct chain between two ether groups, whereby in each alkylene oxide unit, and independent of each other, one of the carbon atoms in α-position to an —O— unit contain either two hydrogen atoms, or one hydrogen atom and one methyl group, and the other carbon atom two hydrogen atoms, whereby each —O— unit carries not more than one methyl group carrying carbon atom in α-position.

This particularly relates to a respective polymer in which the structural element (I) is solely formed by C₂-units based on ethylene oxide and propylene oxide.

Although particularly for a polyalkylene oxide ester polymer with a lower weight average molecular weight M_{w} such as below 1000 g/mol the polymer may alternatively have a cyclic structure, it generally has a non-cyclic structure.

Particularly preferred are the following non-cyclic polyalkylene oxide ester polymers, whereby their radicals and indices relate to the formulas (I) and (Ic):

| | | | |
|---|---|---|---|
| A) | M_{w} [g/mol] | is | 500 to 50 000 |
| | number of ether groups | is | 10 to 560 |
| | number of ester groups | is | 2 to 51 |
| | a | is | 1 |
| | R¹ | is | H |
| | b, c, d, e | are | 0 |
| | a_{xα}, f_{xα} | are | 1 |
| | R¹_{xα}, R⁶_{xα} | are | H |
| | b_{xα}, c_{xα}, d_{xα}, e_{xα} | are | 0 |
| | Xn | is | 4 to 100 |
| B) | M_{w} [g/mol] | is | 500 to 50 000 |
| | number of ether groups | is | 10 to 560 |
| | number of ester groups | is | 2 to 51 |
| | a | is | 1 |
| | R¹ | is | H |
| | b, c, d, e | are | 0 |
| | a_{xα}, f_{xα} | are | 1 |
| | R¹_{xα} | is | H or methyl |
| | R⁶_{xα} | is | H |
| | b_{xα}, c_{xα}, d_{xα}, e_{xα} | are | 0 |
| | Xn | is | 4 to 100 |
| C) | M_{w} [g/mol] | is | 500 to 50 000 |
| | number of ether groups | is | 10 to 560 |
| | number of ester groups | is | 2 to 51 |
| | a | is | 1 |
| | R¹ | is | H |
| | b, c, d, e | are | 0 |
| | a_{xα}, f_{xα} | are | 1 |
| | R¹_{xα} | is | H or ethyl |
| | R⁶_{xα} | is | H |
| | b_{xα}, c_{xα} | are | either both 0 or 1 |
| | R²_{xα}, R³_{xα} | are | H |
| | d_{xα}, e_{xα} | are | 0 |
| | Xn | is | 4 to 100 |
| D) | M_{w} [g/mol] | is | 500 to 50 000 |
| | number of ether groups | is | 10 to 560 |
| | number of ester groups | is | 2 to 51 |
| | a, b, c | are | 1 |
| | R¹, R², R³ | are | H |
| | d, e | are | 0 |
| | a_{xα}, b_{xα}, c_{xα}, f_{xα} | are | 1 |
| | R¹_{xα}, R²_{xα}, R³_{xα}, R⁶_{xα} | are | H |
| | d_{xα}, e_{xα} | are | 0 |
| | Xn | is | 4 to 100 |

Regarding the polyalkylene oxide ester polymers mentioned under B) above, the radicals R¹_{xα} at or near the two borders of the X unit are preferably H, whereas the radicals R¹_{xα} in the rest of unit X are preferably methyl. This is based on the preparation of such elements, which typically start with a polymerization of propylene oxide on which at the end ethylene oxide is co-polymerized.

Regarding the polyalkylene oxide ester polymers mentioned under C) above, the indices b_{xα} and c_{xα} at or near the two borders of the X unit are preferably 0, whereas the indices b_{xα} and c_{xα} in the rest of unit X are preferably 1. This is based on the preparation of such elements, which typically start with a polymerization of 1,2-butylen oxide on which at the end ethylene oxide is co-polymerized.

As already mentioned before, the polyalkylene oxide ester polymer of the invention comprises 10 to 560 ether groups and 2 to 51 ester groups, wherein it contains 1 to 51 structural elements of formula (I). For the avoidance of doubt, it is emphasized that the amount of the ether and ester groups mentioned above refer to the whole polyalkylene oxide ester polymer, including the respective groups present in the structural elements of formula (I). The polyalkylene oxide ester polymer contains preferably ≥15, more preferably ≥20 and particularly preferably ≥30, and preferably ≤500, more preferably ≤400 and particularly preferably ≤350 ether groups. It contains preferably ≥3, more preferably ≥4 and particularly preferably ≥5, and preferably ≤41, more preferably ≤31, particularly preferably ≤21 and very particularly preferably ≤15 ester groups.

The ratio of the number of the ether groups to the number of the ester groups is preferably 4 to 100, more preferably ≥5, particularly preferably ≥10 and very particularly preferably ≥15, and preferably ≤75, more preferably ≤50, particularly preferably ≤40 and very particularly preferably ≤35.

The number of the structural elements (I) in the polyalkylene oxide ester polymer is 1 to 51, preferably ≥2, more preferably ≥3, particularly preferably ≥4 and very particularly preferably ≥5, and preferably ≤41, more preferably ≤31, particularly preferably ≤21, very particularly preferably ≤15 and most preferably ≤9.

The polyalkylene oxide ester polymer can be completely formed by the structural elements (I) plus respective end groups at both ends, containing one or more further alkylene oxide elements, or one or more other structural elements. Preferably, the polyalkylene oxide ester polymer contains further alkylene oxide elements with —O— units and —CO— units at the edges of these elements, forming ester groups together with —CO— units and —O— units of other elements.

Structural elements which form ester groups together with structural element (I) or with other structural elements contain either at least one —O— unit or at least one —CO— unit at one end side of such a structural element. —O— unit and —CO— unit then formally form an ester unit. Structural elements of such further alkylene oxide units preferably contain either two —CO—units or two —O—units at the end of such structural elements. Since an ester group formally requires one —O—unit and one —CO—unit, the number of the structural elements with —O—unit and one —CO—unit at their end shall advantageously be balanced.

As already mentioned above, polyalkylene oxide ester polymer which additionally contain such further structural elements beside structural element (I) are preferred. Specifically, a polyalkylene oxide ester polymer is preferred, which, in addition to structural element (I), further contains 1 to 25 structural elements of the general formula (II)

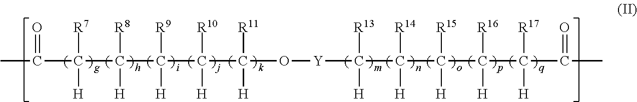

in which
the —CO—unit at the left side is bound to a —O—unit of an adjacent unit of the polymer, forming an ester unit,
the —CO—unit at the right side is bound to a —O—unit of an adjacent unit of the polymer, forming a further ester unit,
R⁷, R⁸, R⁹, R¹⁰, R¹¹, R¹³, R¹⁴, R¹⁵, R¹⁶, R¹⁷ represent independently of each other a hydrogen atom or a C₁₋₁₂ alkyl group,
g, h, i, j, k, m, n, o, p, q represent independent of each other an integer of 0 or 1, whereas the sum of g to k is 1 to 5, and the sum of m to q is 1 to 5, and
Y represents a polyalkylene oxide unit with 0 to 99 alkylene oxide units, whereby the alkylene oxide units contain independent of each other 2 to 6 carbon atoms in the direct chain between two ether groups, whereby each of the carbon atoms in the direct chain between two ether groups contain independent of each other either two hydrogen atoms, or one hydrogen atom and one C₁₋₁₂ alkyl group,
   and polyalkylene oxide units of the general formula (III) in a number suitable to form ester bonds with the —CO—units of the structural elements of the formulas (I) and (II)

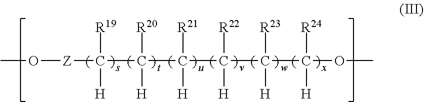

in which
the —O—unit at the left side is bound to a —CO—unit of an adjacent unit of the polymer, forming an ester unit,
the —O—unit at the right side is bound to a —CO—unit of an adjacent unit of the polymer, forming a further ester unit,
R¹⁹, R²⁰, R²¹, R²², R²³, R²⁴ represent independently of each other a hydrogen atom or a C₁₋₁₂ alkyl group,
s, t, u, v, w, x represent independent of each other an integer of 0 or 1, whereas the sum of s to x is 2 to 6, and
Z represents a polyalkylene oxide unit with 0 to 100 alkylene oxide units, whereby the alkylene oxide units are independent of each other and contain 2 to 6 carbon atoms in the direct chain between two ether groups, whereby each of the carbon atoms in the direct chain between two ether groups contain independent of each other either two hydrogen atoms, or one hydrogen atom and one C₁₋₁₂ alkyl group,
   with the proviso that, together with the structural elements of the formula (I), the total number of ester groups does not exceed the maximum number of ester groups specified for the polyalkylene oxide ester polymer.

Each side of the structural element (II) can, for example, be bound to the —O—unit containing side of the structural element (I), to the structural element (III), to an —O—unit containing side of any other polyalkylene oxide, or to another structural element of the polymer which is not represented by any of the structural elements (I), (II) or (III). It is, of course, also possible that one end side of (II) is bound to an end group of the polyalkylene oxide ester polymer. Likewise, each side of the structural element (III) can, for example, be bound to the —CO—unit containing side of the structural element (I), to the structural element (II), to an —CO—unit containing side of any other polyalkylene oxide, or to another structural element of the polymer which is not represented by any of the structural elements (I), (II) or (III). It is, of course, also possible that one end side of (III) is bound to an end group of the polyalkylene oxide ester polymer.

The radicals R⁷, R⁸, R⁹, R¹⁰, R¹¹, R¹³, R¹⁴, R¹⁵, R¹⁶, R¹⁷ in formula (II) represent independent of each other a hydrogen atom or a C₁₋₁₂ alkyl group. The alkyl groups can be linear or in case of C₃₋₁₂ alkyl be linear or branched. Preferred C₁₋₁₂ alkyl groups are methyl, ethyl, n-propyl, iso-propyl, n-butyl, iso-butyl, tert-butyl, n-pentyl, n-hexyl, n-heptyl, n-octyl, n-nonyl, n-decyl, n-undecyl and n-dodecyl. Preferably, R⁷, R⁸, R⁹, R¹⁰, R¹¹, R¹⁴, R¹⁵, R¹⁶, R¹⁷ represent a hydrogen atom, and R¹³ a hydrogen atom or a C₁₋₁₂ alkyl group. More preferably, R¹³ represents a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The indices g, h, i, j, k, m, n, o, p, q in formula (II) represent independent of each other an integer of 0 or 1, whereas the sum of g to k is 1 to 5 and the sum of m to q is 1 to 5. Preferably, i, j, k, m, n, o are 1, and g, h, p, q are 0. More preferably, k, m are 1, and g, h, i, j, n, o, p, q are 0.

A specifically preferred structural element based on formula (II) is the element of the general formula (IIa)

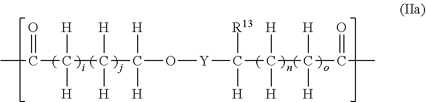

in which
R¹³ represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
i, j, n, o represent an integer of 0 or 1, whereas the sum of i to j is 0 or 2 and the sum of n to o is 0 or 2.

A further specifically preferred structural element based on formula (IIa) is the element of the general formula (IIb)

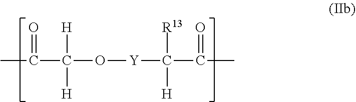

in which
R¹³ represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The unit Y in formula (II) represents a polyalkylene oxide unit with 0 to 99 alkylene oxide units, whereby the alkylene oxide units contain independent of each other 2 to 6 carbon atoms in the direct chain between two —O—units, which can also be denoted as a C₂₋₆ alkylene unit, whereby each of the carbon atoms in the direct chain between two —O—units contain independent of each other either two hydrogen atoms, or one hydrogen atom and one C₁₋₁₂ alkyl group.

The preferred unit Y can be expressed by the general formula (IIc)

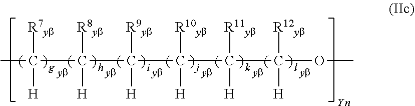

in which
β represents an index from 1 to Yn specifying the running count for each repeating unit,
R⁷_{yγ}, R⁸_{yβ}, R⁹_{yβ}, R¹⁰_{yβ}, R¹¹_{yβ}, R¹²_{yβ} represent independent of each other, and under consideration that β is a running count for each repeating unit, a hydrogen atom or a C₁₋₁₂ alkyl group,
g_{yβ}, h_{yβ}, i_{yβ}, j_{yβ}, k_{yβ}, l_{yβ} represent independent of each other, and under consideration that β is a running count for each repeating unit, an integer of 0 or 1, whereas the sum of g_{yβ} to l_{yβ} is 2 to 6, and
Yn represents an integer of 0 to 99.

The small letter y in the radicals in formula (IIc), as for example in R⁷_{yβ}, and in the indices, as for example in g_{yβ}, indicate that they refer to unit Y. The same is indicated by the capital letter Y in the number of the repeating units Yn. Furthermore, the small letter β in the radicals, as for example in R⁷_{yβ}, and in the indices, as for example in g_{yβ}, specify that each of the radicals and indices have their own sub-number, indicating that the radicals and indices may vary from one alkylene oxide unit to the other within a polyalkylene oxide unit Y. For example, radical R⁷_{y1} of the alkylene oxide unit with the running count 1 may be a hydrogen atom, whereas R⁷_{y2} of the alkylene oxide unit with the running count 2 may be methyl group, and so on. Similarly and also to be understood as an example, the index i_{y1} of the alkylene oxide unit with the running count 1 may be 0, whereas i_{y2} of the alkylene oxide unit with the running count 2 may be 1, and so on.

The C₁₋₁₂ alkyl group in the radicals R⁷_{yβ}, R⁸_{yβ}, R⁹_{yβ}, R¹⁰_{yβ}, R¹¹_{yβ}, R¹²_{yβ} in formula (IIc) can be linear or in case of C₃₋₁₂ alkyl be linear or branched. Preferred C₁₋₁₂ alkyl groups are methyl, ethyl, n-propyl, iso-propyl, n-butyl, iso-butyl, tert-butyl, n-pentyl, n-hexyl, n-heptyl, n-octyl, n-nonyl, n-decyl, n-undecyl and n-dodecyl. Preferably, R⁸_{yβ}, R⁹_{yβ}, R¹⁰_{yβ}, R¹¹_{yβ}, R¹²_{yβ} represent a hydrogen atom, and R⁷_{yβ} a hydrogen atom or a C₁₋₁₂ alkyl group. More preferably, R⁷_{yβ} represents a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The indices g_{yβ}, h_{yβ}, i_{yβ}, j_{yβ}, k_{yβ}, l_{yβ} in formula (IIc) represent independent of each other an integer of 0 or 1, whereas the sum of g_{yβ} to l_{yβ} is 2 to 6. Preferably, g_{yβ}, h_{yβ}, i_{yβ}, l_{yβ} are 1, and j_{yβ}, k_{yβ} are 0. More preferably, g_{yβ}, l_{yβ} are 1, and h_{yβ}, i_{yβ}, j_{yβ}, k_{yβ} are 0.

A specifically preferred unit Y is the unit of the general formula (IId)

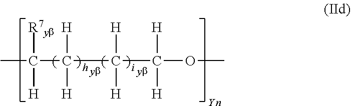

in which
R⁷_{yβ} represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
h_{yβ}, i_{yβ} represent an integer of 0 or 1, whereas the sum of h_{yβ} to i_{yβ} is 0 or 2, and
Yn represents an integer of 0 to 99.

A further specifically preferred unit Y based on formula (IId) is the unit of the general formula (IIe)

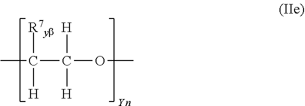

in which
R⁷_{yβ} represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
Yn represents an integer of 0 to 99.

The number of the repeating units Yn in formula (IIc) is an integer of 0 to 99. It is preferably ≥1, more preferably ≥3, particularly preferably ≥7 and very particularly preferably ≥9, and preferably ≤74 and more preferably ≤49.

It is explicitly emphasized that the alkylene oxide units in unit Y can be the same within unit Y or differ from each other, in terms of different radicals R⁷_{yβ} to R¹²_{yβ} and in terms of different indices gyp to lys. This is already clearly indicated by the indexes yβ in formula (IIc) specifying sub-numbers for each radical, as for example in R⁷_{yβ}, and for each index, as for example in g_{yβ}, based on the running count for each repeating unit.

Moreover, it is explicitly emphasized that in the polyalkylene oxide ester polymer also each structural element (II), if more than one element (II) is present, can be identical to one or more of the others, or can differ from one or more of the others.

The radicals R¹⁹, R²⁰, R²¹, R²², R²³, R²⁴ in formula (III) represent independent of each other a hydrogen atom or a C₁₋₁₂ alkyl group. The alkyl groups can be linear or in case of C₃₋₁₂ alkyl be linear or branched. Preferred C₁₋₁₂ alkyl groups are methyl, ethyl, n-propyl, iso-propyl, n-butyl, iso-butyl, tert-butyl, n-pentyl, n-hexyl, n-heptyl, n-octyl, n-nonyl, n-decyl, n-undecyl and n-dodecyl. Preferably, R¹⁹, R²⁰, R²¹, R²², R²³, R²⁴ in formula (III) represent a hydrogen atom, and R¹⁹ a hydrogen atom or a C₁₋₁₂ alkyl group. More preferably, R¹⁹ represents a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The indices s, t, u, v, w, x in formula (III) represent independent of each other an integer of 0 or 1, whereas the sum of s to x is 2 to 6. Preferably, s, t, u, x are 1, and v, w are 0. More preferably, s, x are 1, and t, u, v, w are 0.

A specifically preferred structural element based on formula (III) is the element of the general formula (IIIa)

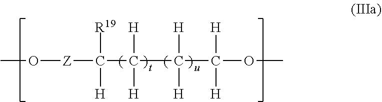

in which
R¹⁹ represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
t, u represent an integer of 0 or 1, whereas the sum of t to u is 0 or 2.

A further specifically preferred structural element based on formula (IIIa) is the element of the general formula (IIIb)

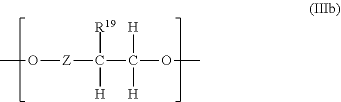

in which
R¹⁹ represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The unit Z in formula (III) represents a polyalkylene oxide unit with 0 to 100 alkylene oxide units, whereby the alkylene oxide units contain independent of each other 2 to 6 carbon atoms in the direct chain between two —O—units, which can also be denoted as a C₂₋₆ alkylene unit, whereby each of the carbon atoms in the direct chain between two —O—units contain independent of each other either two hydrogen atoms, or one hydrogen atom and one C₁₋₁₂ alkyl group.

The preferred unit Z can be expressed by the general formula (IIIc)

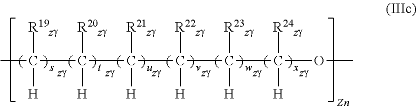

in which
γ represents an index from 1 to Zn specifying the running count for each repeating unit,
R¹⁹_{zγ}, R²⁰_{zγ}, R²¹_{zγ}, R²²_{zγ}, R²³_{zγ}, R²⁴_{zγ} represent independent of each other, and under consideration that γ is a running count for each repeating unit, a hydrogen atom or a C₁₋₁₂ alkyl group,
s_{zγ}, t_{zγ}, u_{zγ}, v_{zγ}, w_{zγ}, x_{zγ} represent independent of each other, and under consideration that γ is a running count for each repeating unit, an integer of 0 or 1, whereas the sum of s_{zγ} to x_{zγ} is 2 to 6, and
Zn represents an integer of 0 to 100.

The small letter z in the radicals in formula (IIIc), as for example in R¹⁹_{zγ}, and in the indices, as for example in s_{zγ}, indicate that they refer to unit Z. The same is indicated by the capital letter Z in the number of the repeating units Zn. Furthermore, the small letter y in the radicals, as for example in R¹⁹_{zγ}, and in the indices, as for example in s_{zγ}, specify that each of the radicals and indices have their own sub-number, indicating that the radicals and indices may vary from one alkylene oxide unit to the other within a polyalkylene oxide unit Z. For example, radical R¹⁹_{z1} of the alkylene oxide unit with the running count 1 may be a hydrogen atom, whereas R¹⁹_{z2} of the alkylene oxide unit with the running count 2 may be methyl group, and so on. Similarly and also to be understood as an example, the index u_{z1} of the alkylene oxide unit with the running count 1 may be 0, whereas u_{z2} of the alkylene oxide unit with the running count 2 may be 1, and so on.

The C₁₋₁₂ alkyl group in the radicals R¹⁹_{zγ}, R²⁰_{zγ}, R²¹_{zγ}, R²²_{zγ}, R²³_{zγ}, R²⁴_{zγ} in formula (IIIc) can be linear or in case of C₃₋₁₂ alkyl be linear or branched. Preferred C₁₋₁₂ alkyl groups are methyl, ethyl, n-propyl, iso-propyl, n-butyl, iso-butyl, tert-butyl, n-pentyl, n-hexyl, n-heptyl, n-octyl, n-nonyl, n-decyl, n-undecyl and n-dodecyl. Preferably, R²⁰_{zγ}, R²¹_{zγ}, R²²_{zγ}, R²³_{zγ}, R²⁴_{zγ} represent a hydrogen atom, and R¹⁹_{zγ} a hydrogen atom or a C₁₋₁₂ alkyl group. More preferably, R¹⁹_{zγ} represents a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom.

The indices s_{zγ}, t_{zγ}, u_{zγ}, v_{zγ}, w_{zγy}, x_{nγ} in formula (IIIc) represent independent of each other an integer of 0 or 1, whereas the sum of s_{zγ} to x_{zγ} is 2 to 6. Preferably, s_{zγ}, t_{zγ}, u_{zγ}, x_{zγ} are 1, and v_{zγ}, w_{zγ} are 0. More preferably, s_{zγ}, x_{zγ} are 1, and t_{zγ}, u_{zγ}, v_{zγ}, w_{zγ} are 0.

A specifically preferred unit Z is the unit of the general formula (IIId)

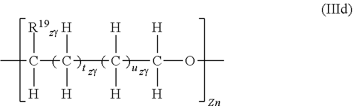

in which
R¹⁹_{zγ} represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
t_{zγ}, u_{zγ} represent an integer of 0 or 1, whereas the sum of t_{zγ} to u_{zγ} is 0 or 2, and
Zn represents an integer of 0 to 100.

A further specifically preferred unit Z based on formula (IIId) is the unit of the general formula (IIIe)

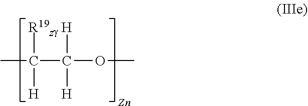

in which
R¹⁹_{zγ} represents a hydrogen atom or a C₁₋₁₂ alkyl group, more preferably a hydrogen atom, methyl, ethyl, n-propyl, iso-propyl, n-butyl, iso-butyl, tert-butyl, n-pentyl, n-hexyl, n-heptyl, n-octyl, n-nonyl, n-decyl, n-undecyl or n-dodecyl, particularly preferably a hydrogen atom, methyl, ethyl, n-propyl or n-decyl, very particularly preferably a hydrogen atom or methyl, and most preferably a hydrogen atom, and
Zn represents an integer of 0 to 100.

The number of the repeating units Zn in formula (IIIc) is an integer of 0 to 100. It is preferably ≥2, more preferably ≥4, particularly preferably ≥8 and very particularly preferably ≥10, and preferably ≤75 and more preferably ≤50.

It is explicitly emphasized that the alkylene oxide units in unit Z can be the same within unit Z or differ from each other, in terms of different radicals R¹⁹_{zγ} to R²⁴_{zγ} and in terms of different indices s_{zγ} to x_{zγ}. This is already clearly indicated by the indexes zγ in formula (IIIc) specifying sub-numbers for each radical, as for example in R¹⁹_{zγ}, and for each index, as for example in s_{zγ}, based on the running count for each repeating unit.

Moreover, it is explicitly emphasized that in the polyalkylene oxide ester polymer also each structural element (III), if more than one element (III) is present, can be identical to one or more of the others, or can differ from one or more of the others.

The different parts of the structural elements (II) and (III) such as the radicals, indices and the units X and Y including their general and preferred values have already been described above. The following paragraphs relate to preferred specific combinations of these parts.

Particularly preferred is a polyalkylene oxide ester polymer containing the structural elements (I), (II) and (III) in which
R¹³, R¹⁹ represent independent of each other a hydrogen atom or a methyl group,
R⁹, R¹⁰, R¹¹, R¹⁴, R¹⁵, R²⁰, R²¹, R²⁴ represent a hydrogen atom,
g, h, p, q, v, w are 0,
k, m, s, x are 1,
i, j, n, o, t, u represent independent of each other an integer of 0 or 1, whereas the sum of i to j is 0 or 2, the sum of n to o is 0 or 2, and the sum of t to u is 0 or 2, and
Y represents a polyalkylene oxide unit with 3 to 99 alkylene oxide units, and Z represents a polyalkylene oxide unit with 4 to 100 alkylene oxide units, whereby the alkylene oxide units are independent of each other and contain 2 or 4 carbon atoms in the direct chain between two ether groups, whereby in each alkylene oxide unit, and independent of each other, one of the carbon atoms in α-position to an —O—unit contain either two hydrogen atoms, or one hydrogen atom and one methyl group, and the remaining other one or three carbon atoms two hydrogen atoms each, whereby each —O—unit carries not more than one methyl group carrying carbon atom in α-position.

This particularly relates to a respective polymer in which the structural elements (II) and (III) are solely formed by C₂-units, solely formed by C₄-units or formed by a mixture thereof. Each C₂—and C₄-unit may either carry only hydrogen atoms, or hydrogen atoms and one methyl group. If a methyl group in a C₂—or C₄-unit is present, it is bound at a carbon atom in α-position to an —O—unit, whereby each —O—unit carries in its α-position not more than one methyl group carrying carbon atom.

Such elements are typically based on ethylene oxide monomers, propylene oxide monomers, tetrahydrofuran monomers or mixtures thereof. In case of propylene oxide based —CHCH₃—CH₂—O—and tetrahydrofuran based —CH₂—CH₂—CH₂—CH₂—O— units within formulas (II) and (III), it may be advantageous if the structural elements (II) and (III) contain at the border region of the respective element one or more C₂-based units without methyl groups. This can be easily achieved by firstly polymerizing propylene oxide or tetrahydrofuran and later stop the addition of propylene oxide and tetrahydrofuran, respectively, and supply ethylene oxide to finish the polymerization, leading to C₂-based units at both end sides. The obtained polyalkylene oxides can then be processed as described further down to form the structural units (II) and (III) in the polyalkylene oxide ester polymer.

Due to the mentioned production process, ethylene oxide is co-polymerized with propylene oxide and tetrahydrofuran, respectively, causing an irregular structure in the border region in which —CHCH₃—CH₂—O—and —CH₂—CH₂—CH₂—CH₂—O—units, respectively, alternate with —CH₂—CH₂—O—units, so that the transition from —CHCH₂—CH₂—O—and —CH₂—CH₂—CH₂—CH₂—O—units, respectively, to —CH₂—CH₂—O—units might not be sharp. Such effect is well known in the art and the respective alternating structure also often called “dirty structures”.

Another particularly preferred polyalkylene oxide ester polymer contains the structural elements (I), (II) and (III) in which
R¹³, R₁₉ represent independent of each other a hydrogen atom or a methyl group,
R⁹, R¹⁰, R¹¹, R¹⁴, R¹⁵, R²⁰, R²¹, R²⁴ represent a hydrogen atom,
g, h, i, j, n, o, p, q, t, u, v, w are 0,
k, m, s, x are 1, and
Y represents a polyalkylene oxide unit with 3 to 99 alkylene oxide units, and Z represents a polyalkylene oxide unit with 4 to 100 alkylene oxide units, whereby the alkylene oxide units are independent of each other and contain 2 carbon atoms in the direct chain between two ether groups, whereby in each alkylene oxide unit, and independent of each other, one of the carbon atoms in α-position to an —O—unit contain either two hydrogen atoms, or one hydrogen atom and one methyl group, and the remaining other carbon atom two hydrogen atoms, whereby each —O—unit carries not more than one methyl group carrying carbon atom in α-position.

This particularly relates to a respective polymer in which the structural elements (II) and (III) are solely formed by C₂-units based on ethylene oxide and propylene oxide.

The number of the structural elements (II) in the polyalkylene oxide ester polymer is 1 to 25, preferably ≥2, more preferably ≥3, particularly preferably ≥4, very particularly preferably ≥5 and most preferably ≥6, and preferably ≤23, more preferably ≤22, particularly preferably ≤20 and very particularly preferably ≤17. The total number of the elements (I) and (II) is adapted such that the total number of the ester groups does not exceed the maximum number of the ester groups specified for the polyalkylene oxide ester polymer.

Since the formation of ester groups by the elements (I), (II) and (III) require an —O—unit at the edge of one element and a —CO—unit at the edge of the other element, the total number of such —O—units and —CO— units in the elements from which the polyalkylene oxide ester polymer is formed is preferably adjusted such that the intended amount of ester groups are formed. A possible surplus of —O—units or —CO— units can, for example, be bound to end groups or to other structural element of the polymer. Since the numbers of —O—units and —CO— units in element (I) are already balanced and element (II) only provides —CO—units, element (III) is preferably present in a number suitable to form ester bonds with the —CO—units of the structural elements of the formulas (I) and (II). More preferably, the ratio between the number of elements (II) and the number of elements (III) is 0.8 to 1.2, particularly preferably ≥0.9 and very particularly preferably ≥0.95, and particularly preferably ≤1.1 and very particularly preferably ≥1.05, and most preferably 1. The surplus of —O—units or —CO— units can, for example, be bound to end groups or other structural elements.

Since the polyalkylene oxide ester polymers used in the invention are advantageously produced by esterification of monomers, it is favorable to use monomers which are easily available. Suitable monomers are particularly monomers which already comprise the structural element (I), in which, for example, the monomer of the element (I) contains a hydroxy group at the one edge, as a precursor for the —O—unit, and a carboxylic acid, carboxylic acid alkyl ester or carboxylate (such as for example —COONa) group at the one edge, as a precursor for the —CO—unit. Even though monomers of element (I) can be prepared in a high amount and high purity, it is easier to obtain a mixture of monomers of elements (I), (II) and (III). Consequently, polyalkylene oxide ester polymers based on such a mixture contain the elements (I), (II) and (III). Based on the composition of such monomer mixtures, a polyalkylene oxide ester polymer with a ratio of the number of the structural elements (I) to the number of the structural elements (II) of 0.5 to 8 is preferred, a ratio of 0.7 to 6 more preferred and a ratio of 0.85 to 4.7 particularly preferred.

As already mentioned before, the polyalkylene oxide ester polymer used in the invention can, beside end groups, also contain further polyalkylene oxide elements other than (I), (II) and (III), or even other structural elements. Further polyalkylene oxide elements other than (I), (II) and (III) may, for example, be elements with an alkylene unit which has more than six carbon atoms in the direct chain between two ether groups. Other structural elements can, for example, be based on diols other than structural element (III), on dicarboxylic acids other than structural element (II), or on alpha-hydroxy-omega carboxylic acids other than structural element (I), e.g. sebacic acid or terephthalic acid. The structural elements (I), (II) and (III) generally constitute 50 to 100%, preferably 70 to 100%, more preferably 80 to 100%, particularly preferably 90 to 100%, very particularly preferably 95 to 100%, and most preferably 98 to 100% of the number average molecular weight Mn of the polyalkylene oxide ester polymer. The nature of the structural elements, and thus the composition of the polyalkylene oxide ester polymer can, for example, be determined by hydrolyzing the ester bonds and analyzing the structural units with common analytical methods such as gas chromatography, HPLC, NMR and the like.

Due to the presence of end groups at both sides of the polymer, the amount of the structural elements (I), (II) and (III) is typically at least slightly below 100% of the number average molecular weight Mn of the polyalkylene oxide ester polymer, even if the polymer does not contain other elements than (I), (II) and (III). However, particularly in case of —OH groups as end groups, the numerical effect due to the very low molecular mass of the hydrogen atom compared with the molecular mass of the polyalkylene oxide ester polymer is so small that a 100% value can be achieved under consideration of the accuracy of the analytical measurement.

Although elements other than the elements (I), (II) and (III) may be present in the polyalkylene oxide ester polymer, it preferably contains only the elements (I), (II) and (III) plus two end groups.

Particularly preferred polyalkylene oxide ester polymers based on the structural elements (I), (II) and (III) are the following polymers, whereby their radicals and indices relate to the formulas (I), (Ic), (II), (IIc), (III) and (IIIc):

| | | | |
|---|---|---|---|
| A) | M_{w} [g/mol] | is | 500 to 50 000 |
| | number of ether groups | is | 10 to 560 |
| | number of ester groups | is | 2 to 51 |
| | a | is | 1 |
| | R¹ | is | H |
| | b, c, d, e | are | 0 |
| | a_{xα}, f_{xα} | are | 1 |
| | R¹_{xα}, R⁶_{xα} | are | H |
| | b_{xα}, c_{xα}, d_{xα}, e_{xα} | are | 0 |
| | Xn | is | 4 to 100 |
| | k, m | are | 1 |
| | R¹¹, R¹³ | are | H |
| | g, h, i, j, n, o, p, q | are | 0 |
| | g_{yβ}, l_{yβ} | are | 1 |
| | R⁷_{yβ}, R¹²_{yβ} | are | H |
| | h_{yβ}, i_{yβ}, j_{yβ}, k_{yβ} | are | 0 |
| | Yn | is | 3 to 99 |
| | s, x | are | 1 |
| | R¹⁹, R²⁴ | are | H |
| | t, u, v, w | are | 0 |
| | s_{zγ}, x_{zγ} | are | 1 |
| | R¹⁹_{zγ}, R²⁴_{zγ} | are | H |
| | t_{zγ}, u_{zγ}, v_{zγ}, w_{zγ} | are | 0 |
| | Zn | is | 4 to 100 |
| | ratio of number of (I)/number of | is | 0.5 to 8 |
| | (II) | | |
| | Percentage of molecular weight of | is | 95 to 100 |
| | (I) + (II) + (III) based on number | | |
| | average molecular weight Mₙ of | | |
| | polyalkylene oxide ester polymer | | |
| | End groups | | — OH or — COOH |
| B) | M_{w} [g/mol] | is | 500 to 50 000 |
| | number of ether groups | is | 10 to 560 |
| | number of ester groups | is | 2 to 51 |
| | a | is | 1 |
| | R¹ | is | H |
| | b, c, d, e | are | 0 |
| | a_{xα}, f_{xα} | are | 1 |
| | R¹_{xα} | are | H or methyl |
| | R⁶_{xα} | is | H |
| | b_{xα}, c_{xα}, d_{xα}, e_{xα} | are | 0 |
| | Xn | is | 4 to 100 |
| | k, m | are | 1 |
| | R¹¹, R¹³ | are | H |
| | g, h, i, j, n, o, p, q | are | 0 |
| | g_{yβ}, l_{yβ} | are | 1 |
| | R⁷_{yβ} | are | H or methyl |
| | R¹²_{yβ} | is | H |
| | h_{yβ}, i_{yβ}, j_{yβ}, k_{yβ} | are | 0 |
| | Yn | is | 3 to 99 |
| | s, x | are | 1 |
| | R¹⁹, R²⁴ | are | H |
| | t, u, v, w | are | 0 |
| | s_{zγ}, x_{zγ} | are | 1 |
| | R¹⁹_{zγ} | are | H or methyl |
| | R²⁴_{zγ} | is | H |
| | t_{zγ}, u_{zγ}, v_{zγ}, w_{zγ} | are | 0 |
| | Zn | is | 4 to 100 |
| | ratio of number of (I)/number of | is | 0.5 to 8 |
| | (II) | | |
| | Percentage of molecular weight of | is | 95 to 100 |
| | (I) + (II) + (III) based on number | | |
| | average molecular weight Mₙ of | | |
| | polyalkylene oxide ester polymer | | |
| | End groups | | —OH or —COOH |
| C) | M_{w} [g/mol] | is | 500 to 50 000 |
| | number of ether groups | is | 10 to 560 |
| | number of ester groups | is | 2 to 51 |
| | a | is | 1 |
| | R¹ | is | H |
| | b, c, d, e | are | 0 |
| | a_{xα}, f_{xα} | are | 1 |
| | R¹_{xα} | are | H or ethyl |
| | R⁶_{xα} | is | H |
| | b_{xα}, c_{xα}, d_{xα}, e_{xα} | are | 0 |
| | Xn | is | 4 to 100 |
| | k, m | are | 1 |
| | R¹¹, R¹³ | are | H |
| | g, h, i, j, n, o, p, q | are | 0 |
| | g_{yβ}, l_{yβ} | are | 1 |
| | R⁷_{yβ} | are | H or ethyl |
| | R¹²_{yβ} | is | H |
| | h_{yβ}, i_{yβ}, j_{yβ}, k_{yβ} | are | 0 |
| | Yn | is | 3 to 99 |
| | s, x | are | 1 |
| | R¹⁹, R²⁴ | are | H |
| | t, u, v, w | are | 0 |
| | s_{zγ}, x_{zγ} | are | 1 |
| | R¹⁹_{zγ} | are | H or ethyl |
| | R²⁴_{zγ} | is | H |
| | t_{zγ}, u_{zγ}, v_{zγ}, w_{zγ} | are | 0 |
| | Zn | is | 4 to 100 |
| | ratio of number of (I)/number of | is | 0.5 to 8 |
| | (II) | | |
| | Percentage of molecular weight of | is | 95 to 100 |
| | (I) + (II) + (III) based on number | | |
| | average molecular weight Mₙ of | | |
| | polyalkylene oxide ester polymer | | |
| | End groups | | —OH or —COOH |
| D) | M_{w} [g/mol] | is | 500 to 50 000 |
| | number of ether groups | is | 10 to 560 |
| | number of ester groups | is | 2 to 51 |
| | a, b, c | are | 1 |
| | R¹, R², R³ | are | H |
| | d, e | are | 0 |
| | a_{xα}, b_{xα}, c_{xα}, f_{xα} | are | 1 |
| | R¹_{xα}, R²_{xα}, R³_{xα}, R⁶_{xα} | are | H |
| | d_{xα}, e_{xα} | are | 0 |
| | Xn | is | 4 to 100 |
| | i, j, k, m, n, o | are | 1 |
| | R⁹, R¹⁰, R¹¹, R¹³, R¹⁴, R¹⁵ | are | H |
| | g, h, p, q | are | 0 |
| | g_{yβ}, h_{yβ}, i_{yβ}, l_{yβ} | are | 1 |
| | R⁷_{yβ}, R⁸_{yβ}, y⁹_{yβ}, R¹²_{yβ} | are | H |
| | j_{yβ}, k_{yβ} | are | 0 |
| | Yn | is | 3 to 99 |
| | s, t, u, x | are | 1 |
| | R¹⁹_{zγ}, R²⁰_{zγ}, R²¹_{zγ}, R²⁴ | are | H |
| | v, w | are | 0 |
| | s_{zγ}, t_{zγ}, u_{zγ}, x_{zγ} | are | 1 |
| | R¹⁹_{zγ}, R²⁰_{zγ}, R²¹_{zγ},R²⁴_{zγ} | are | H |
| | v_{zγ}, w_{zγ} | are | 0 |
| | Zn | is | 4 to 100 |
| | ratio of number of (I)/number of | is | 0.5 to 8 |
| | (II) | | |
| | Percentage of molecular weight of | is | 95 to 100 |
| | (I) + (II) + (III) based on number | | |
| | average molecular weight Mn of | | |
| | polyalkylene oxide ester polymer | | |
| | End groups | | —OH or —COOH |

Regarding the polyalkylene oxide ester polymers mentioned under B) above, the radicals R¹_{xα}, R⁷_{yβ} and R¹⁹_{zγ} at or near the two borders of the X, Y and Z units are preferably H, whereas the radicals R¹⁹_{xα}, R⁷_{yβ} and R¹⁹_{zγ} in the rest of the X, Y and Z units are preferably methyl. This is based on the preparation of such elements, which typically start with a polymerization of propylene oxide on which at the end ethylene oxide is co-polymerized.

Regarding the polyalkylene oxide ester polymers mentioned under C) above, the radicals R¹_{xα}, R⁷_{yβ} and R¹⁹_{zγ} at or near the two borders of the X, Y and Z units are preferably H, whereas the radicals R¹_{xα}, R⁷_{yβ} and R¹⁹_{zγ} in the rest of the X, Y and Z units are preferably ethyl. This is based on the preparation of such elements, which typically start with a polymerization of 1,2-butylene oxide on which at the end ethylene oxide is co-polymerized.

Both general structure definitions of PAG-ester/polyalkylene oxide ester polymers specified above as separate embodiments for use as the polymer backbone A for the graft polymers of this invention are an integral part of this invention; it is specifically pointed out that both definitions overlap to a large extent, with the second structure definition being more comprehensive and being defined in organic chemistry terms, whereas the first structure definition is more narrow and uses the language of polymer chemistry; this latter polymer language is also intended to serve for a clearer understanding of those readers more fluent in polymer chemistry terms than in organic chemistry terms. Nevertheless, graft polymers of the present invention include both structures, and it is by no means intended to limit the invention to either one of the structure definitions; moreover, both are embodiments of the polymer backbone (A) of this present invention.

The polyalkylene oxide ester polymer/PAG-ester polymer of the invention can easily be prepared by esterification of blocks of the respective structural elements of which the polymer is to be build, whereas the blocks to be esterified contain, if intended as end group in the polyalkylene oxide ester polymer, at least one esterifiable end group in the block, and, if intended as a middle group in the polyalkylene oxide ester polymer, two esterifiable end groups in the block. In principle, as esterifiable end groups, basically groups typically known as esterifiable groups can be used. However, esterifiable end groups forming the —O—part in the later —COO—ester group are, for example, —OH, and esterifiable end groups forming the —CO—part in the later —COO—ester group are, for example, —COOH, —COOR in which R is a hydrocarbon group with 1 to 12 C-atoms, as for example a —COOCH3 group, or carboxylates in which the cation are preferably alkali metals such as sodium or potassium, and preferably —COOH and —COONa.

For the sake of completeness, it is explicitly mentioned that in principal also polyalkylene oxide ester polymers with a weight average molecular weight Mw higher than 50 000 g/mol, such as 100 000 g/mol or even much higher, can easily be prepared by esterification of the respective blocks.

In that context, a preferred process for the preparation of a polyalkylene oxide ester polymer of the invention was found, in which polyalkylene oxide comprising the structural element (I) and having one primary OH and one COOH end group, or a mixture of such polyalkylene oxides, is esterified at a temperature of 50 to 250° C. and a pressure of 0.1 kPa abs to 1 MPa abs in the presence of an esterification catalyst.

Polyalkylene oxides comprising the structural element (I) and having one primary OH and one COOH end group can be synthesized in different ways. One possibility is to partially oxidize the respective polyalkylene oxide having two primary OH end groups and to separate the polyalkylene oxide component having one primary OH and one COOH end group (called “monoacid” also called “PAG-MC” for “PAG-mono carbonic acid”) for example by vacuum distillation from the unconverted polyalkylene oxide having two OH end groups (called “diol”, “PAG-DO” for “PAG-diol”) and the fully oxidized polyalkylene oxide having two COOH end groups (called “diacid”; also “PAG-DC” for “PAG-dicarbonic acid”). Another possibility is to specifically synthesize the polyalkylene oxide having one primary OH and one COOH end group, for example, by adding sodium metal and bromoacetic acid to polyalkylene oxide and processing the obtained sodium carboxylate end group to the respective carboxylic acid end group. However, both ways are elaborate in terms of their process steps, be it by a vacuum distillation or complex synthesis steps, but they may be justified if a polyalkylene oxide ester polymer with a high content of structural unit (I) is wanted.

Furthermore, another and more preferred process for the preparation of the polyalkylene oxide ester polymer of the invention was found, in which
a) a polyalkylene oxide comprising the structural element (I) and having one primary OH and one COOH end group, or a mixture of such polyalkylene oxides,
b) a polyalkylene oxide comprising the structural element (II) and having two COOH end groups, or a mixture of such polyalkylene oxides, and
c) a polyalkylene oxide comprising the structural element (III) and having two primary OH end groups, or a mixture of such polyalkylene oxides,
   are esterified at a temperature of 50 to 250° C. and a pressure of 0.1 kPa abs to 1 MPa abs in the presence of an esterification catalyst.

It was further recognized according to the invention that a mixture of the components a) to c) can easily be produced by partial oxidation of the respective polyalkylene oxide having two primary OH end groups. Such partial oxidation is described further below. For the sake of completeness, it is mentioned that the mixture of the components a) to c) can, of course, also be prepared by mixing of the individual components.

As the ester groups in the polyalkylene oxide ester polymer are typically formed by esterification of polyalkylene oxide blocks having esterifiable end groups, and each ester group requires one —O—containing end group such as an —OH group, and one —CO—containing end group such as a —COOH group, it is favorable that their amounts are equal or approximately equal. A small surplus of one type can, however, be absorbed by elements other than (I), (II) and (III) which are able to link to the —O—or —CO— groups. Additionally, the two end groups of the polyalkylene oxide ester polymer may also bind two of such groups. Based on this, the ratio of the number of the OH end groups to the number of the COOH end groups is preferably 0.9 to 1.1, more preferably ≥0.95, particularly preferably ≥0.98 and more particularly preferably ≥0.99, and more preferably ≤1.05, particularly preferably ≤1.02 and more particularly preferably ≤1.01.

The esterification of the respective polyalkylene oxide blocks can generally be performed in a manner industrially known and, for example, described in U.S. Pat. No. 6,310,235 or 5,324,853. The educts are esterified in the presence of an esterification catalyst and the educts are preferably provided such that the ratio of the number of the OH end groups to the number of the COOH end groups is within the objected range.

Usually, different types of esterification catalysts can be used. They can roughly be divided into acidic, amphoteric and basic catalysts. As representatives of acidic catalysts, mineral acids such as sulfuric acid and phosphoric acids, and organic sulfonic acids such as methanesulfonic acid and p-toluenesulfonic acid, trifluormethansulfonsgure are mentioned. Further acidic catalysts can also be acidic solids such as zeolites, especially Ti-zeolites, diverse oxides, mixed metal oxides, sulfated oxides, acid ion exchange resins, protonic heteropolyoxoanions, salts of heteropolyoxoanions, acid clays and phosphates. Representatives of basic catalysts are, for example, ZnO, La₂O₃, ThO₂, ZrO₂, hydrotalcites, hydroxyapatites, alkali metal oxides, alkaline earth metal oxides, basic zeolites and solid superbases like Verkade bases or guanidines. As possible amphoteric catalysts oxides of zinc(II), tin (II) and tin(IV) are mentioned. Furthermore, lewis acidic catalysts derived from group 4 metal cations of the Periodic Table of the Elements, e.g. Ti and Zr compounds, such as Ti(VI) and Zr(IV), derived from group 3 metal cations of the Periodic Table of the Elements, e.g. Sc(III) compounds, or group 5 metal cations of the Periodic Table of the Elements, e.g. Al(III) compounds, are also useful. However, also catalysts containing metal cations of group 12 and 15 of the Periodic Table of the Elements, such as Sn(IV), Sn(II), Zn(II) and Bi(III) are mentioned. The corresponding anions can typically be chosen from alkoxylates such as isopropoxylates and isobutyrates alkanoate, aralkylcarboxylates, halogen, sulfate, organic sulfonates such as p-toluolsulfonates or methansulfonate, amidomethansulfonate, trifluormethansulfonate or triflourmethansulfonimide.

The esterification catalysts are typically used in a customary amount in the range of 0.02 to 10 wt.-%, preferably ≥0.05 wt.-% and more preferably ≥0.1 wt.-%, and preferably ≤5 wt.-% and more preferably ≤2 wt.-%, based on the sum of the compounds to be esterified.

The esterification can be carried out in the absence or in the presence of a solvent. If it is carried out in the presence of a solvent, an organic solvent that is inert under the reaction conditions is preferably used. These include, for example, aliphatic hydrocarbons, halogenated aliphatic hydrocarbons, aromatic and substituted aromatic hydrocarbons or ethers. Preferably, the solvent is selected from pentane, hexane, heptane, ligroin, petroleum ether, cyclohexane, benzene, toluene, xylene, chlorobenzene, dichlorobenzenes, dibutyl ether, tetrahydrofuran, dioxane and mixtures thereof. Suitable solvents forming an azeotrope with water are aromatic hydrocarbons, e.g. benzene, alkyl aromates, toluene or xylenes. Also halogenated compounds with suitable high boiling points are useful.

The esterification is effected at a temperature of 50 to 250° C., preferably ≥70° C. and more preferably ≥80° C., and preferably ≤220° C. and more preferably ≤200° C. If the esterification catalyst is an organic acid or mineral acid, the esterification is usually carried out at a temperature range of 50 to 160° C. If the esterification catalyst is a metal containing catalyst, the esterification is usually carried out at a temperature range of 80 to 250° C. With regard to the pressure, the esterification can be performed at a wide pressure range from vacuum to a pressure above atmospheric pressure, ranging from 0.1 kPa abs to 1 MPa abs. Preferably, it is performed at ≤0.5 MPa abs and more preferably at ≤0.2 MPa abs.

The esterification can take place in the absence or in the presence of an inert gas. An inert gas is generally understood as meaning a gas which, under the stated reaction conditions, does not enter into any reactions with the starting materials, reagents, solvents or the resulting products involved in the reaction.

Suitable reactors for performance of the process of the esterification are in principle all reactors suitable for esterification reactions. Examples include stirred tanks.

Mainly depending on the nature of the educt, the type and amount of the catalyst and the reaction temperature, the esterification typically requires a reaction time of 1 to 24 hours, more typically 2 to 12 hours.

The obtained polyalkylene oxide ester polymer is typically but not necessarily worked up, depending on the intended purity. In case of a workup, components other than the polyalkylene oxide ester polymer are usually removed, particularly the esterification catalysts. Polyalkylene oxide ester polymer free of esterification catalysts are generally important for the product quality. Heterogenous catalysts can typically be removed by physical methods like filtration or centrifugation. Homogenous catalysts can typically be removed by the use of stationary ionic exchanging units.

In a preferred embodiment the catalyst is not removed but left in the polyalkylene oxide polymer. This embodiment is favorable in case the amounts of the catalyst employed are at the low to very low end of the range disclosed herein.

The molecular mass of the polyalkylene oxide ester polymer can be easily adjusted by the ratio of the OH to COOH end groups of the compounds to be esterified. Simply spoken, the more the ratio deviates from an exact 1:1 ratio, the fewer ester groups are formed in polyalkylene oxide ester polymer. Practically, this can, for example, be done by addition of a diol component or a diacid component, ideally of the one of the educts to the reaction mixture. However, the addition of other monools or monocarboxylic acid fulfill also the purpose.

The end groups of the polyalkylene oxide compounds which are not esterified with other polyalkylene oxide compounds typically form the end groups of the polyalkylene oxide ester polymer of the invention. Thus, not esterified —OH end groups of the polyalkylene oxide compounds lead to —OH end groups of the polyalkylene oxide ester polymer and not esterified —COOH, —COOR or —COOM end groups of the polyalkylene oxide compounds to —COOH, —COOR or —COOM end groups of the polyalkylene oxide ester polymer, whereby R is typically a hydrocarbon group with 1 to 12 C-atoms such as a methyl group, and M typically an alkali metal such as sodium or potassium.

For the sake of completeness, it is mentioned that beside the blocks containing the structural elements (I), or the structural elements (I) to (III), also other elements with esterifiable end groups can be present, particularly if a polyalkylene oxide ester polymer is to be produced which also shall contain such other elements. Examples of such other elements are polyalkylene oxide elements with an alkylene unit which has more than six carbon atoms in the direct chain between two ether groups.

As already mentioned before, mixtures of the components a) to c) comprising the so called “monoacid”, “diacid” and “diol” can easily be produced by partial oxidation of the respective polyalkylene oxide having two primary OH end groups (“diol”). This also enables the way to obtain polyalkylene oxide comprising the structural element (I) and having one primary OH and one COOH end group (the so called “monoacid”), or a mixture of such polyalkylene oxides, in a higher concentration by separating off at least parts of the other components (the so called “diacid” and “diol”), if a polyalkylene oxide ester polymer with a higher content of the structural element (I) is sought and thus increases the flexibility.

In a preferred process, the polyalkylene oxide used in the esterification comprising the structural element (I) and having one primary OH and one COOH end group, or a mixture of such polyalkylene oxides, has been produced by partial oxidation of the respective polyalkylene oxide having two primary OH end groups, or a mixture of such polyalkylene oxides, with oxygen at a temperature of 20 to 100° C. and a partial oxygen pressure of 0.01 to 2 MPa abs in the presence of water and a heterogeneous catalyst comprising platinum, palladium or gold.

Depending on the intended composition and structure of the polyalkylene oxide ester polymer, the polyalkylene oxide comprising the structural element (I) and having one primary OH and one COOH end group, or a mixture of such polyalkylene oxides, which has been prepared by partial oxidation as described above, can, of course, be blended with other components such as polyalkylene oxide comprising the structural element (II) and having two COOH end groups, polyalkylene oxide comprising the structural element (III) and having two OH end groups, any other polyalkylene oxides having OH and/or COOH groups, or any other esterifiable structural elements.

In a more preferred process, the mixture of the components a) to c) used in the esterification has been produced by partial oxidation of the respective polyalkylene oxide having two primary OH end groups, or a mixture of such polyalkylene oxides, with oxygen at a temperature of 20 to 100° C. and a partial oxygen pressure of 0.01 to 2 MPa abs in the presence of water and a heterogeneous catalyst comprising platinum, palladium or gold, and the oxidation reaction been stopped after a ratio of the number of the OH end groups to the number of the COOH end groups in the range of 0.9 to 1.1 has been reached.

The respective polyalkylene oxide having two primary OH end groups to be used as starting material for the partial oxidation can easily be prepared by methods known in the art. The oxidation of a OH end group to a COOH end group requires the existence of a primary OH group. For example, polyethylene oxides can advantageously be prepared by polymerization of ethylene oxide. Similarly, polypropylene oxides and poly-1,2-butylene oxides can advantageously be prepared by polymerization of propylene oxide and 1,2-butylene oxide, respectively, but due to the existence of a secondary OH group at one end, ethylene oxide is usually copolymerized at the end of the polymerization to form ethylene oxide units at the outer region, whereas the inner region comprises propylene oxide and 1,2-butylene oxide units, respectively. Furthermore, polytetrahydrofuran can advantageously be prepared by polymerization of tetrahydrofuran.

The partial oxidation process is conducted in the presence of water. Water promotes the oxidation of the —OH end groups into —COOH end groups in various ways. For instance, water, in the case of use of a suspension catalyst, improves the suspension thereof in the reaction mixture and additionally also lowers the viscosity of the reaction mixture. The concentration of water in the liquid phase is preferably kept at 50 to 95 wt.-%, preferably ≥60 wt.-%, and preferably ≤90 wt.-% and more preferably ≤80 wt.-%.

The catalyst used in the partial oxidation process is a heterogeneous catalyst comprising platinum, palladium or gold, and preferably platinum as active component. Typically, the active metals are fixed on a support. A wide variety of different materials may be used as support. Examples include inorganic oxides, for instance aluminum oxide, zirconium oxide, titanium dioxide, silicon oxide, inorganic silicates, for instance aluminum silicate, or charcoal. It is of course also possible to use mixtures of different support materials. Preference is given to the use of charcoal as support.

The preferred catalyst with platinum as active component comprises generally 0.1% to 10% by weight, preferably ≥0.5% by weight, more preferably ≥1% by weight and even more preferably ≥4% by weight, and preferably ≤8% by weight and more preferably ≤6% by weight, of platinum, based in each case on the total mass of the heterogeneous catalyst. More preferably, a heterogeneous catalyst comprising 1 to 10 wt.-% and particularly 4 to 10 wt.-% of platinum on charcoal is used.

The catalyst to be used may also comprise further metals as well as platinum, palladium or gold. The term “further metals” is understood to mean metals from the fourth to sixth periods of groups 3 to 16 of the Periodic Table of the Elements, beginning with scandium (atomic number 21) and ending with polonium (atomic number 84). Preferably, the total content of further metals is 0 to 100 wt.-%, preferably 0 to 30 wt.-%, more preferably 0 to 10 wt.-%, even more preferably 0 to 1 wt.-% and especially 0 to 0.1 wt.-%, based on the mass of platinum. In particular, the total content of cadmium, lead and bismuth is preferably 0 to 1 wt.-%, more preferably 0 to 0.5 wt.-%, especially preferably 0 to 0.1 wt.-%, even more preferably 0 to 0.05 wt.-% and especially 0 to 0.01 wt.-%, based on the mass of platinum. The catalyst is thus preferably prepared without deliberate addition of further metals.

The heterogeneous supported catalyst can be used in various geometric shapes and sizes, for instance as powder or shaped bodies. Pulverulent catalysts may be operated, for example, in suspension mode. In the case of a fixed bed mode, preference is given to using shaped bodies, for example pellets, cylinders, hollow cylinders, spheres or extrudates or tablets. The shaped bodies in that case are typically fixed in the reactor by the known methods. In the case of shaped catalyst bodies, these preferably have an average particle size of 1 to 10 mm.

However, preference is given to using the catalyst in the form of a powder. In that case, the pulverulent catalyst is in suspension in the reactor. In order to prevent discharge from the reaction system, a filter is typically used here to retain the suspension catalyst. One example of a customarily used filter is the crossflow filter.

Irrespective of the geometric shape and size of the catalyst particles, the platinum is generally in the form of particles having an average diameter of 0.1 to 50 nm, measured via x-ray diffraction. However, there may also be smaller or larger particles.

In the production of the heterogeneous supported catalyst, the platinum is generally applied to the support by suitable methods such as those described in US 2020/017,745.

The heterogeneous supported catalyst generally has a BET surface area of ≥1 m2/g and ≤10 000 m2/g, determined to DIN ISO 9277:2014-01. When carbon is used as support, the BET surface area is preferably in the range of ≥500 m2/g and ≤10 000 m2/g.

The preferred platinum based catalyst is typically applied in an amount of 0.1 to 50 mg platinum per g of the polyalkylene oxide to be partially oxidized, preferably ≥1 mg and preferably ≤20 mg.

Since the polyalkylene oxide feedstock is pH-neutral in aqueous solution, the pH on commencement of the oxidation is typically at or close to 7. As a result of the formation of the COOH groups, there is a gradual fall in the pH, and so there is generally a value of 1 or 3 toward the end of the oxidation.

However, the partial oxidation can also be performed in the presence of a base, such as sodium or potassium hydroxide. A basic condition increases the oxidation potential and leads to the formation of carboxylic acid salts (carboxylates) instead of carboxylic acids. As already mentioned before in the description of the esterification process, also carboxylates can be directly used in the esterification.

In case of the absence of basic compounds, there is direct formation of the carboxylic acids. This saves (i) the use of additional chemicals (base and extraneous acid), and (ii) the disposal of the salt formed from the base and the extraneous acid.

The oxidation medium used in the partial oxidation process is molecular oxygen. Oxygen is added either in pure form or diluted with other gases, for example in the form of air or an O2/N2 mixture. Preferably an oxygen gas having a content of ≥90% by volume, more preferably of ≥95% by volume, even more preferably of ≥99% by volume and especially of ≥99.5% by volume is used. The use of very highly concentrated or pure oxygen makes it possible to keep the amount of offgas relatively small.

In order to promote the distribution of the oxygen in the reactor, it may be advantageous to meter it in in the form of fine bubbles, for example through a frit.

The partial oxygen pressure present in the oxidation is 0.01 to 2 MPa, preferably ≥0.02 MPa and more preferably ≥0.05 MPa, and preferably ≤1 MPa and more preferably ≤0.3 MPa.

The oxidation is effected at a temperature of 20 to 100° C., preferably ≥30° C. and more preferably ≥40° C., and preferably ≤80° C. and more preferably ≤70° C.

Suitable reactors for performance of the partial oxidation process are in principle all reactors suitable for the performance of exothermic gas/liquid reactions. Examples include stirred tanks, trickle bed reactors and bubble column reactors. In order to remove the heat of reaction, the reactors are typically equipped with a cooling device. According to the reactor type and nature of the catalyst, the cooling device advantageously comprises cooling elements within the reactor or cooling elements in an external circuit outside the reactor. For example, a stirred tank preferably comprises internal cooling elements, whereas it is more advantageous in a bubble column, for example, to integrate the cooling elements in the external circuit.

If the catalyst is in the form of a shaped body, this is typically fixed in the reactor in the form of a fixed bed. For this purpose, the trickle bed reactor is a particularly useful option, in which the catalyst can be introduced in the form of a bed. But it is possible to use shaped catalyst bodies in a stirred tank reactor. In this case, it is then advantageous to fix the shaped catalyst bodies in a compartment, for example a wire basket.

In the case of the preferred use of a pulverulent catalyst, it is advantageously in suspended form in the reaction mixture. Preferred reactors for the purpose are, for example, stirred tanks or bubble columns. In order to prevent the pulverulent catalyst from settling out, corresponding mixing of the liquid reaction mixture is required. In a stirred tank, this is typically achieved by use of a stirrer. In the case of a bubble column, the mixing is usually achieved via an external circuit with a conveying pump.

In principle, the bubble column, with regard to the liquid circuit, can be operated either in upward direction or in downward direction, but operation in downward direction is typically more advantageous.

In the partial oxidation process, either semicontinuous or continuous operation is possible. In both cases, the oxygen, for assurance of the desired partial pressure, is fed to the reactor continuously or at least intermittently, but preferably continuously.

In the semicontinuous mode of operation, prior to commencement of the reaction, the reactor is initially charged with the complete amount of aqueous reactant mixture together with catalyst and, during the oxidation reaction, no fresh reactant is supplied, nor is any liquid reaction mixture withdrawn. The reactor is not emptied until after the oxidation reaction has ended.

In the continuous mode of operation, there is likewise liquid reaction mixture together with catalyst present in the reactor, but there is constant withdrawal of a small amount of liquid reaction and supply of corresponding amount of aqueous reactant. If a suspension catalyst has been used here, the liquid reaction mixture is advantageously removed from the reactor via a filter device, for example a crossflow filter.

As the partially oxidized polyalkylene oxide shall in any event contain polyalkylene oxide having one primary OH and one COOH end group, the oxidation reaction has to be conducted in a way that a portion of the primary OH groups remains unoxidized whereas others are already oxidized to COOH groups. This can easily be achieved by stopping the oxidation reaction at a degree of oxidation in which the desired amount of the partially oxidized polyalkylene oxide is present. Except for low molecular weight polyalkylene oxides with a molecular weight of only a very few hundred g/mol, the probability that a primary OH group is oxidized to a COOH group is independent of whether the other end group of the polyalkylene oxide has already been oxidized or not. At the beginning of the oxidation, polyalkylene oxide having one primary OH and one COOH end group (called “monoacid”) is primarily formed. With an increasing amount of it, the probability that also the other OH group is oxidized increases, so that then also polyalkylene oxide having two COOH end groups (called “diacid”) is formed.

In case of a semicontinuous mode of operation, the easiest way to stop a further oxidation is to timely stop the supply of oxygen. The oxygen present in the reactor would at least be consumed. Additional actions which can be combined with such a stop of the oxygen supply are, for example, the additional supply of an inert gas in order to replace a part of the oxygen in the reactor, the lowering of the total pressure in the reactor which automatically causes a decrease of the partial oxygen pressure, or the cooling of the reaction liquid, e.g. by withdrawing it from the reactor through a cooling unit. However, the most effective measure is to stop the oxygen supply.

Such an interference in the progress of the oxidation reaction is very easily controllable since the oxidation reaction takes many hours to proceed, so that there is a sufficiently long time frame in which the “monoacid” is present in a large concentration. The time frame in which the partial oxidation reaction is to be stopped, can be determined in various ways. Firstly, the required time for the partial oxidation under specified conditions, such as temperature, oxygen partial pressure, nature and amount of catalyst, etc., can be determined in preliminary tests in which the oxidation is stopped at different times and the reaction product analyzed for its composition. The oxidation time which is required for obtaining the intended composition can then be estimated. Another possibility to control the partial oxidation is to measure the amount of oxygen provided to the reactor as a measure of the oxygen absorbed by the oxidation. The degree of oxidation can then be calculated by the amount of OH groups present in the educt polyalkylene oxide and the stoichiometry of their oxidation to COOH groups. A further way is to take samples over time and to analyze them, e.g. by determining the acid number by titration as a measure of the COOH groups already formed. And last but not least, also physical in-situ measurements like the measurement of the electrical conductivity, of the dielectric constant or of the impedance are mentioned, which, of course, have to be calibrated in advance.

In case of a continuous mode of operation, the degree of oxidation can easily be adjusted by the residence time of the mixture in the reactor under reaction conditions.

As already mentioned above, oxidation reaction takes many hours. For a partial oxidation of around 50% of the OH groups, the typical reaction time is around 3 to 20 hours, preferably ≥4 hours and more preferably ≥5 hours, and preferably ≤18 hours and more preferably ≤15 hours.

For the sake of completeness, it is to mention that beside the oxidation of the OH groups to COOH groups as the main reaction also an oxidative degradation takes place in a small degree. In such an oxidative degradation a small amount of alkylene oxide units may be totally oxidized. This inevitably causes a small decrease of the medium chain length of the partially oxidized alkylene oxide compared with the educt alkylene oxide before the partial oxidation.

After the end of the reaction, the reaction mixture is typically removed from the reactor and separated from the catalyst. In the case of use of a suspension catalyst, the removal is sensibly by filtration. Alternatively, it is also possible to allow the suspension catalyst to settle out at the reactor base after the end of the reaction and to remove the supernatant liquid. It is also possible to separate the catalyst by use of a centrifuge. The catalyst removed can generally be reused without further workup. The water or at least a major portion of the water is typically removed by distillation, for example over a wiped film evaporator. The partially oxidized polyalkylene oxide can then be used in the esterification step.

The polyalkylene oxide ester polymer of the invention can broadly substitute conventional polyalkylene oxide polymers in their applications.

The products containing such polyalkylene oxide ester polymers or produced with such polyalkylene oxide ester polymers are significantly better biodegradable than similar products containing, or produced with conventional polyalkylene oxide polymers.

The polyalkylene oxide ester polymer as employed as polymer backbone A in the invention is a new class of polymers which are able to substitute polyalkylene oxides, particularly polyethylene oxides, polypropylene oxides, poly-1,2-butylene oxides and polytetrahydrofuran, in their typical applications such as for the encapsulation of fragrances, and in the preparation of graft polymers for its use in homecare and laundering applications. Some of the application properties of the polyalkylene oxide ester polymers are even better than those of the conventional polyalkylene oxides. The greatest advantage of the new type of polymers is their significantly better biodegradability which can be an important contribution in protecting the environment, particularly since the polyalkylene oxide ester polymers can be easily substitute the conventional polyalkylene oxides in homecare and laundering applications. The polyalkylene oxide ester polymer are safe and durable in their applications.

Moreover, the polyalkylene oxide ester polymer of the invention can be easily produced in high yields from easily available starting materials in a two-step process.

The same applies to the graft polymers based on such polyalkylene oxide ester polymers, the graft polymers being detailed in the following:

### Graft Polymers

The graft polymer of the invention includes a polymer backbone (A) as defined before by the two structure definitions of PAG-ester/polyalkylene oxide ester polymers, and polymeric side chains (B) attached to the polymer backbone, with the polymeric sidechains (B) comprising at least one vinyl ester monomer (B1) and optionally at least one olefinically unsaturated monomer (B2) other than the monomer (B1), wherein preferably at least 10 weight percent of the total amount of vinyl ester monomer (B1) is preferably selected from vinyl acetate, vinyl propionate and vinyl laurate, more preferably from vinyl acetate and vinyl laurate, and most preferably vinyl acetate, and wherein the remaining amount of vinyl ester may be any other known vinyl ester, but most preferably such other vinyl ester is not present.

In respect of the polymeric sidechains (B) contained within the graft polymer according to the present invention, it is preferred that the polymeric sidechains (B) are obtained by radical polymerization of at least one vinyl ester monomer (B1) and optionally at least one olefinically unsaturated monomer (B2) other than the monomer (B1), wherein preferably at least 10 weight percent of the total amount of vinyl ester monomer (B1) is chosen from vinyl acetate and vinyl propionate, more preferably vinyl acetate, and wherein the remaining amount of vinyl ester may be any other known vinyl ester.

It is preferred, that as vinyl ester only vinyl acetate and/or vinyl propionate, and more preferably at least 80 weight percent, even more preferably at least 90 weight percent, and most preferably essentially only (i.e. about 100 wt. % or even 100 wt. %) vinyl acetate is employed as vinyl ester.

As vinyl ester monomer (B1) any further vinyl ester besides vinyl acetate or vinyl propionate may be employed which are known to a person skilled in the art, such as vinyl valerate, vinyl pivalate, vinyl laurate, vinyl neodecanoate (such as VEOVA9 and VEOVA 10), vinyl decanoate or vinyl benzoate. The vinyl ester is preferably selected from vinyl acetate, vinyl propionate and vinyl laurate, more preferably from vinyl acetate and vinyl laurate, and most preferably is vinyl acetate.

As the optionally at least one olefinically unsaturated monomer (B2) other than the monomer (B1) in principle any monomer polymerizable with monomer (B1) can be employed, such as those defined as B2a) and B2b) below-Monomers B2a) are chosen from
N-vinyllactams, such as N-vinylpyrrolidone, N-vinylpiperidone, N-vinylcaprolactam, derivatives thereof substituted with C1- to C8-alkyl groups, such as 3-methyl-, 4-methyl- or 5-methyl-N-vinylpyrrolidone;
N-Vinylamides, such as N-vinylformamide and the N-vinylamine thereof obtainable following polymerization by hydrolysis, N-vinyl-N-methylacetamide, and derivatives thereof.

Preferred monomers B2a) are vinyllactams such as N-vinylpyrrolidone, 3-methyl-N-vinylpyrrolidone, 4-methyl-N-vinylpyrrolidone, 5-methyl-N-vinylpyrrolidone, N-vinylpiperidone and N-vinylcaprolactam. More preferred monomers B2a) are N-vinylpyrrolidone, N-vinylcaprolactam.

Particularly preferred monomer B2a) is N-vinylpyrrolidone.

Suitable monomers B2b) are:

Salts, esters and amides of carboxylic acids such as acrylic acids and derivatives thereof, such as substituted acrylic acids, where the substituents are on the carbon atoms in the 2- or 3-position of the acrylic acid and are selected independently of one another from the group consisting of C1-C4-alkyl, —CN and —COOH. Preferred salts are salts of those acids with alkanolamines such as ethanolamine.

These monomers B2b) include, for example:
acrylic acids such as acrylic acid itself or anhydride thereof, methacrylic acid, ethylacrylic acid, 3-cyanoacrylic acid, maleic acid, fumaric acid, crotonic acid, maleic anhydride or half-ester thereof, itaconic acid or half-ester thereof;
acrylamides such as acrylamide itself, N-methylacrylamide, N,N-dimethylacrylamide, N-ethylacrylamide, N-1-propylacrylamide, N-2-propylacrylamide, N-butylacrylamide, N-2-butylacrylamide, N-t-butylacrylamide, N-octylacrylamide, N-t-octylacrylamide, N-octadecylacrylamide, N-phenylacrylamide, N-dodecylacrylamide, laurylacrylamide, stearylacrylamide, N-2-hydroxyethylacrylamide, N-3-hydroxypropylacrylamide, N-2-hydroxypropylacrylamide;
methacrylamides such as methacrylamide itself, N-methylmethacrylamide, N,N-dimethylmethacrylamide, N-ethylmethacrylamide, N-1-propylmethacrylamide, N-2-propylmethacrylamide, N-butylmethacrylamide, N-2-butylmethacrylamide, N-t-butylmethacrylamide, N-octylmethacrylamide, N-t-octylmethacrylamide, N-octadecylmethacrylamide, N-phenylmethacrylamide, N-dodecylmethacrylamide, N-laurylmethacrylamide, stearyl(meth)acrylamide, N-2-hydroxyethyl(meth)acrylamide, N-3-hydroxypropyl(meth)acrylamide, N-2-hydroxypropyl(meth)acrylamide;
further amides such as ethacrylamide, maleimide, fumaric acid monoamide, fumaric diimide;
aminoalkyl(meth)acrylamides such as (dimethylamino)methyl(meth)acrylamide, 2-(dimethylamino)ethyl(meth)acrylamide, 2-(dimethylamino)propyl(meth)acrylamide, 2-(diethylamino)propyl(meth)acrylamide, 3-(dimethylamino)propyl(meth)acrylamide, 3-(diethylamino)propyl(meth)acrylamide, 3-(dimethylamino)butyl(meth)acrylamide, 4-(dimethylamino)butyl(meth)acrylamide, 8-(dimethylamino)octyl(meth)acrylamide, 12-(dimethylamino)dodecyl(meth)acrylamide, or analogs thereof quaternized on the amine with, for example, methyl chloride, ethyl chloride, dimethyl sulfate or diethyl sulfate, such as, for example, 3-(trimethylammonium)propyl(meth)acrylamide chloride;
acrylates such as C1-C18-alkyl acrylates such as methyl acrylate, ethyl acrylate, n-propyl acrylate, isopropyl acrylate, n-butyl acrylate, isobutyl acrylate, t-butyl acrylate, 2-ethylhexyl acrylate, decyl acrylate, dodecyl acrylate, lauryl acrylate, stearyl acrylate, 2,3-dihydroxypropyl acrylate, 2-hydroxyethyl acrylate, 2-hydroxypropyl acrylate, 3-hydroxypropyl acrylate, 2,3-dihydroxypropyl acrylate, 2-methoxyethyl acrylate, 2-methoxypropyl acrylate, 3-methoxypropyl acrylate, 2-ethoxyethyl acrylate, 2-ethoxypropyl acrylate, 3-ethoxypropyl acrylate, glyceryl monoacrylate, alkylene glycol acrylates or polyalkylene glycol acrylates having in total 2 to 200 EO and/or PO units and/or EO/PO units with hydroxy, amino, carboxylic acid, sulfonic acid or alkoxy group such as methoxy or ethoxy groups on the end of the chain, where “EO” means “ethylene oxide” and “PO” means propylene oxide;
methacrylates such as C1-C18-alkyl methacrylates, such as methyl methacrylate, ethyl methacrylate, n-propyl methacrylate, isopropyl methacrylate, n-butyl methacrylate, isobutyl methacrylate, t-butyl methacrylate, 2-ethylhexyl methacrylate, decyl methacrylate, dodecyl methacrylate, stearyl methacrylate, 2,3-dihydroxypropyl methacrylate, 2-hydroxyethyl methacrylate, 2-hydroxypropyl methacrylate, 3-hydroxypropyl methacrylate, 2,3-dihydroxypropyl methacrylate, 2-methoxyethyl methacrylate, 2-methoxypropyl methacrylate, 3-methoxypropyl methacrylate, 2-ethoxyethyl methacrylate, 2-ethoxypropyl methacrylate, 3-ethoxypropyl methacrylate, glyceryl monomethacrylate, and also alkylene glycol methacrylates or polyalkylene glycol methacrylates having in total 2 to 200 EO and/or PO units and/or EO/PO units with hydroxy, amino, carboxylic acid, sulfonic acid or alkoxy group such as methoxy or ethoxy groups on the end of the chain;
ethacrylates such as C1-C18-alkyl ethacrylates, such as methyl ethacrylate, ethyl ethacrylate, n-butyl ethacrylate, isobutyl ethacrylate, t-butyl ethacrylate, 2-ethylhexyl ethacrylate, decyl ethacrylate, 2-hydroxyethyl ethacrylate, 2-methoxyethyl acrylate, 2-methoxyethyl ethacrylate, 2-ethoxyethyl ethacrylate;
amino-C1-C18-alkyl (meth)acrylates such as N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate or analogs thereof quatemized on the amine with for example methyl chloride, ethyl chloride, dimethyl sulfate or diethyl sulfate;
alkyl esters such as uniform or mixed diesters of maleic acid with methanol, ethanol, 1-propanol, 2-propanol, n-butanol, 2-butanol, tert-butanol, alkylene glycol or polyalkylene glycol having in total 2 to 200 EO and/or PO units and/or EO/PO units with hydroxy, amino, carboxylic acid, sulfonic acid or alkoxy group such as methoxy or ethoxy groups on the end of the chain;
alkyl esters of C1-C40 linear, C3-C40 branched-chain or C3-C40 carbocyclic carboxylic acids;
vinyl ethers, such as methyl, ethyl, butyl or dodecyl vinylethers;
ethers of allyl alcohol and polyethylene oxide and/or propylene oxide and/or poly(ethylene oxide-co-propylene oxide) having in total 2 to 200 EO and/or PO units and/or EO/PO units with hydroxy, amino, carboxylic acid, sulfonic acid or alkoxy groups such as methoxy or ethoxy groups on the end of the chain;
N-vinyloxazolines such as N-vinyloxazoline, N-vinylmethyloxazoline, N-vinylethyloxazoline, N-vinylpropyloxazoline, N-vinylbutyloxazoline, N-vinylphenyloxazoline;
halides such as vinyl halides or allyl halides, such as vinyl chloride, allyl chloride, vinylidene chloride;
olefinically unsaturated hydrocarbons such as hydrocarbons having at least one carbon-carbon double bond, such as styrene, alpha-methylstyrene, tert-butyistyrene, butadiene, isoprene, cyclohexadiene, ethylene, propylene, 1-butene, 2-butene, isobutene, vinyttoluene;
sulfonic acids such as unsaturated sulfonic acids, such as, for example, acrylamidopropanesulfonic acid, more preferably their salts, for example styrene sulfonate;
methyl vinyl ketone, vinylfuran, allyl alcohol.

Preferred monomers B2b) are salts and esters of those acids, particularly preferred of acrylic acid and methacrylic acid.

Very particularly preferred monomers B2b) are esters of those acids, particularly preferred of acrylic acid and methacrylic acid, and more particularly preferred esters with C1 to C10-alkanols, preferably C1 to C6-alkanols.

In a particularly preferred embodiment as monomers B2 only B2a-monomer(s) is(are) present; in a even more preferred embodiment only N-vinylpyrrolidone is present as monomer B2.

In case at least one optional further monomer (B2), preferably a B2a-monomer only, more preferably a vinyllactam and most preferably only N-vinylpyrrolidone, is employed for preparing the polymeric sidechains (B) within the graft polymers according to the present invention, the ratio of the mandatory vinyl ester monomer (B1) versus said further monomer (B2) may have any value known to a person skilled in the art; however, the amount of vinyl ester monomer (B1) is usually not smaller than 1% by weight (in relation to the sum of (B1) and (B2)). By consequence, the polymeric sidechains (B) may be obtained by, preferably, radical polymerization of 1 to 100% by weight, more preferably 30 to 100 wt. %, of monomer (B1), which is most preferably vinyl acetate, and 0 to 99% by weight of that B2-monomer, most preferably N-vinylpyrrolidone, and more preferably of 0 to 70 weight percent, as optional further monomer (B2).

It is preferred within the context of the present invention that the polymeric sidechains (B) are obtained by free radical polymerization of
(B1) 30 to 100% by weight (in relation to the sum of (B1) and (B2)) of at least one vinyl ester monomer (B1), preferably 60 to 100% by weight, more preferably 80 to 100% by weight, which is preferably selected from vinyl acetate, vinyl propionate and vinyl laurate, more preferably from vinyl acetate and vinyl laurate, and most preferably vinyl acetate, and
(B2) 0 to 70% by weight (in relation to the sum of (B1) and (B2)) of at least optional further monomer (B2), preferably a B2a-monomer only, more preferably a vinyllactam and most preferably only N-vinylpyrrolidone, as further monomer (B2), preferably 0 to 40% by weight, more preferably 0 to 20% by weight.

The graft polymer of the invention therefore consists of a polymer backbone (A) as defined before by the two structure definitions of PAG-ester/polyalkylene oxide ester polymers as described in detail above, and polymeric side chains (B) attached to the polymer backbone, with the polymeric sidechains (B) being
(B1) 30 to 100% by weight (in relation to the sum of (B1) and (B2)) of at least one vinyl ester monomer (B1), preferably 60 to 100% by weight, more preferably 80 to 100% by weight, which is preferably selected from vinyl acetate, vinyl propionate and vinyl laurate, more preferably from vinyl acetate and vinyl laurate, and most preferably vinyl acetate, and
(B2) 0 to 70% by weight (in relation to the sum of (B1) and (B2)) of at least optional further monomer (B2), preferably a B2a-monomer only, more preferably a vinyllactam and most preferably only N-vinylpyrrolidone, as further monomer (B2), preferably 0 to 40% by weight, more preferably 0 to 20% by weight.

The graft polymers of the invention may contain a certain amount of ungrafted polymers (“ungrafted side chains”) made of vinyl ester(s), e.g. polyvinylacetate in case only vinyl acetate is employed, and/or—when further monomers are employed—homo- and copolymers of vinyl ester(s) and the other monomers. The amount of such ungrafted vinylacetate-homo- and copolymers may be high or low, depending on the reaction conditions, but is preferably to be lowered and thus low. By this lowering, the amount of grafted side chains is preferably increased. Such lowering can be achieved by suitable reaction conditions, such as dosing of vinyl ester and radical initiator and their relative amounts and also in relation to the amount of backbone being present. This is generally known to a person of skill in the present field. Keeping such amounts of vinyl ester-polymers not being grafted low, also improves the clarity of the polymer solution, as especially homopolymers of vinyl esters such as vinyl acetate are known to impart cloudiness in aqueous solutions when present in amounts as low as 500 ppm.

The inventive graft polymers maybe characterized by their degree of grafting (number of graft sites of the polymeric sidechains (B) on the polymer backbone (A)). The degree of graft may be high or low, depending on the reaction conditions. Preferably, the degree of grafting is low.

This adjustment of the degree of grafting and the amount of ungrafted polymers can be used to optimize the performance in areas of specific interest, e.g. certain (e.g. detergent-) formulations, application areas or desired cleaning etc. performance.

It is even more preferred within the context of the present invention that the polymeric sidechains (B) are obtained by radical polymerization of 100% by weight (in relation to the total amount of monomers employed) of at least one vinyl ester monomer (B1), which is preferably selected from vinyl acetate, vinyl propionate and vinyl laurate, more preferably from vinyl acetate and vinyl laurate, and most preferably vinyl acetate.

Within the context of the present invention, it is more preferred that no other monomers besides the at least one vinyl ester monomer (B1) and the optionally present at least optional further monomer (B2), preferably a B2a-monomer only, more preferably a vinyllactam and most preferably only N-vinylpyrrolidone, as optional further monomer (B2) are employed within the respective polymerization process for obtaining the polymeric sidechains (B).

However, in a preferred embodiment if any further polymeric monomers besides the monomers according to (B1) and the optionally preferred N-vinyllactame (B2a) are present, such monomers (other than B1 and the at least one N-vinyllactame as B2a) are present in an amount of less than 1% of the total amount of monomers employed for obtaining the polymeric sidechains (B). Most preferably, the amount of said additional monomers is less than 0.5% by weight, even more preferably less than 0.01% by weight, most preferably, there is no additional monomer present besides the monomers (B1) and the optionally at least optional further monomer (B2), preferably a B2a-monomer only, more preferably a vinyllactam and most preferably only N-vinylpyrrolidone, as (B2).

Inventive graft polymers have at least one of the following properties, preferably two or more, to be successfully employed in the various fields of applications targeted with this present invention:
a) Biodegradation of a certain level, such biodegradation being tested as defined elsewhere within this specification. To exhibit a commercially useful biodegradation the percentage of biodegradation should be at least 25 percent, preferably at least 30%, more preferably at least 40%, and even more preferably at least 60%, such as 35, 45, 55, 60, 65, 75, 80, 85 or more up to 100% (all percentages in weight % based on the total solid content) within 56 days, and most preferably any of the before percentages and preferred, more preferred etc. percentages within 28 days.
b) Water-solubility of the polymers should be present to a certain extent, to be able to employ the polymers within the aqueous environment typically present in the fields of applications as generally targeted with this present invention. Preferably, inventive polymers should exhibit a medium to good, more preferably a good solubility in the environment of an aqueous formulation as typically employed in such fields for the various kinds of formulations, e.g. dish washing, automatic dish-washing, hard surface cleaning, fabric cleaning, fabric care, agrochemical formulations etc.
c) Viscosities of the polymer solutions should be such that at reasonably high solid concentrations of the polymer as to be handled in and after production and to be provided to the user, which could be e.g. as a “pure” (then typically liquid) product, dissolved in a solvent, typically an aqueous solution containing water and organic solvents, only water or only organic solvents, the viscosity of such polymer or polymer solution being in a range that allows typical technical process steps such as pouring, pumping, dosing etc. Hence, the viscosities should be preferably in a range of about up to less than 4000 mPas, more preferably up to 3500 mPas, even more preferably up to 3000 mPas, such as up to 4500, 3750, 3250, 2750 or even 2600 or below such as 2500, 2000, 1750, 1500, 1250, 1000, 750, 500, 250, 200, 150, or 100 mPas, at concentrations of the polymer (based on the total solid content of the polymer in solution, as defined by weight percent of the dry polymer within the total weight of the polymer solution) of preferably at least 10 wt. %, more preferably at least 20, and even more preferably at least 40 wt. %, and most preferably at least 50 wt. %, such as at least 60, 70, 80 or even 90 wt. %. The viscosity may be measured at either 25° C. or at elevated temperature, e.g. temperatures of 50 or even 60° C. By this a suitable handling of the polymer solutions in commercial scales is possible. It is of course evident that depending on the amount of solvent being added the viscosity is lower when the amount of solvent increases and vice versa, thus allowing for adjustment in case desired. It is also evident that the viscosity being measured depends on the temperature at which it is being measured, e.g. the viscosity of a given polymer with a given solid content of e.g. 80 wt. % will be higher when measured at lower temperature and lower when measured at a higher temperature. In a preferred embodiment the solid content is in between 70 and 99 wt. %, more preferably in between 75 and 85 wt. %, with no additional solvent being added but the polymer as prepared. In a more preferred embodiment, the solid content is in between 70 and 99 wt. %, more preferably in between 75 and 95 wt. %, with no additional solvent being added but the polymer as prepared, and the viscosity is lower than 3000 mPas, more preferably 3250, or even below 2750, 2600, 2500, 2000, 1750, 1500, 1250, 1000, 750, 500 or even 250 mPas, when measured at 60° C.

To achieve these requirements, the following guidance can be given on how to achieve such properties of the inventive graft polymers:

Biodegradability increases generally with at least one of the following conditions:
lower molecular weight of the polymer backbone (A) compared to higher molecular weight;
lower weight percentage of polymeric side chains (monomers B) being grafted onto the backbone compared to higher weight percentages;

As further criteria of course the individual performance of a specific graft polymer needs to be evaluated and thus ranked for each individual formulation in a specific field of application. Due to the broad usefulness of the graft polymers an exhaustive overview is not possible, but the present specification and examples provide a guidance on how to prepare and select useful graft polymers of specifically desired properties and how to tune the properties to the desired needs.

One such criteria for the area of home care and especially fabric care of course it the performance upon washing, e.g. subjecting a certain material exhibiting stains of certain materials to a defined washing procedure.

The examples give some guidance for the application for washing of fabrics, i.e. the general area of fabric care.

The examples on agrochemical formulation also give a guidance on how such graft polymers can be employed to obtain useful stable formulations of agrochemical actives.

Likewise, other active ingredients from other field could be formulated in similar and analogous ways following the overall guidance given herein. The straight-forward approach for such use of the inventive graft polymers is of course the initial replacement of conventional graft polymers of similar composition but being based on conventional polyalkylene glycols, and then fine-tune the properties of the inventive graft polymers to the specific needs following the teachings given herein.

Depending on the individual needs for a polymer exhibiting a defined degree of biodegradation, water solubility and viscosity (i.e. handling properties) the general and specific teachings herein—without being intended to be limited to the specific examples being given—will guide on how to obtain such polymer.

### Process of Production of Graft Polymer

Another subject-matter of the present invention is a process for preparing the inventive graft polymers as described above. Within this process for obtaining at least one graft polymer according to the present invention, at least one monomer (B1) and optionally at least one further monomer (B2), preferably a B2a-monomer only, more preferably a vinyllactam and most preferably only N-vinylpyrrolidone, (B2) are polymerized in the presence of at least one block copolymer backbone (A).

The graft polymer of the invention therefore if prepared from using a polymer backbone (A) as defined before by the two structure definitions of PAG-ester/polyalkylene oxide ester polymers, and attaching polymeric side chains (B) to the polymer backbone by radical polymerization, by using the following monomers in the given amounts:
(B1) 30 to 100% by weight (in relation to the sum of (B1) and (B2)) of at least one vinyl ester monomer (B1), preferably 60 to 100% by weight, more preferably 80 to 100% by weight, and
(B2) 0 to 70% by weight (in relation to the sum of (B1) and (B2)) of at least one optionally at least one further monomer (B2), preferably a B2a-monomer only, more preferably a vinyllactam and most preferably only N-vinylpyrrolidone, as further monomer (B2), preferably 0 to 40% by weight, more preferably 0 to 20% by weight.

It has to be noted that the grafting process as such, wherein a polymeric backbone, such as a polyethylene glycol-polymer backbone, is grafted with polymeric sidechains, is in principle known to a person skilled in the art. Any process known to the skilled person in this respect can in principle be employed within the present invention.

Within the process of the present invention, it is preferred that the polymeric sidechains (B) are obtained by radical polymerization.

The radical polymerization as such is also known to a skilled person. The person skilled in the art also knows that the inventive process can be carried out in the presence of a radical-forming initial (C) and/or at least one solvent (D). The skilled person knows the respective components as such.

The term “radical polymerization” as used within the context of the present invention comprises besides the free radical polymerization also variants thereof, such as controlled radical polymerization. Suitable control mechanisms are RAFT, NMP or ATRP, which are each known to the skilled person, including suitable control agents.

It is even more preferred that a process according to the present invention is carried out by a method comprising the polymerization of at least one monomer (B1) selected from vinyl esters, preferably from vinyl acetate, vinyl propionate, vinyl laurate, more preferably vinyl acetate and vinyl laurate, most preferably vinyl acetate, and optionally one or more further monomer (B2) selected from olefinically unsaturated monomers polymerizable with the monomers B1, preferably selected from monomers B2a, more preferably N-vinyllactams, more preferably only N-vinylpyrrolidone, in order to obtain the polymer sidechains (B) in the presence of at least one PAG-ester backbone (A), a free radical-forming initiator (C) and, if desired, up to 50% by weight, based on the sum of components (A), (B1), optionally (B2), and (C) of at least one organic solvent (D), at a mean polymerization temperature at which the initiator (C) has a decomposition half-life of from 40 to 500 min, in such a way that the fraction of unconverted graft monomers (B1) and optionally (B2) and initiator (C) in the reaction mixture is constantly kept in a quantitative deficiency relative to the PAG-ester backbone (A).

The amount of ((free) radical-forming) initiator (C) may be any amount generally known, but is preferably from 0.1 to 5% by weight, in particular from 0.3 to 3.5% by weight, based in each case on the polymeric sidechains (B).

For the process according to the invention, it is preferred that the steady-state concentration of radicals present at the mean polymerization temperature is substantially constant and the graft monomers (B1) or (B2) are present in the reaction mixture constantly only in low concentration (for example of not more than 5% by weight). This allows the reaction to be controlled, and graft polymers can be prepared in a controlled manner with the desired low polydispersity.

To assure a safe temperature control although a large or all amounts of the monomers are present from the start of the polymerization temperature, it is advisable, and thus preferred, to use an additional and efficient measure to control the temperature. This can be done by external or internal cooling; such cooling can be done by internal or external coolers, such as heat exchangers, or using reflux condensors when working at the boiling temperature of the solvent or the solvent mixture.

When a constantly low concentration of radicals and monomers is maintained, such temperature control may not be a crucial issue—however depending on the scale the polymerization is performed, with much higher criticality at larger scales—, as the temperature is at least partially controlled also by the propagation of the polymerization reaction by controlling the radical concentration and the available amount of polymerizable monomers.

Of course, depending on the scale of the polymerisation reaction, such additional cooling as described before may become necessary when the scale gets large enough that the ratio from volume to surface of the polymerization mixture becomes very large. This however is generally known to a person of skill in the art of commercial scale polymerisations, and thus can be adapted to the needs.

The term “mean polymerization temperature” is intended to mean here that, although the process is substantially isothermal, there may, owing to the exothermicity of the reaction, be temperature variations which are preferably kept within the range of +/−10° C., more preferably in the range of +/−5° C.

According to the invention, the (radical-forming) initiator (C) at the mean polymerization temperature should have a decomposition half-life of from 40 to 500 min, preferably from 50 to 400 min and more preferably from 60 to 300 min.

According to the invention, the initiator (C) and the graft monomers (B2) and/or (B2) are advantageously added in such a way that a low and substantially constant concentration of undecomposed initiator and graft monomers (B1) and/or (B2) is present in the reaction mixture. The proportion of undecomposed initiator in the overall reaction mixture is preferably <15% by weight, in particular <10% by weight, based on the total amount of initiator metered in during the monomer addition.

The mean polymerization temperature is appropriately in the range from 50 to 140° C., preferably from 60 to 120° C. and more preferably from 65 to 110° C.

Examples of suitable initiators (C) whose decomposition half-life in the temperature range from 50 to 140° C. is from 20 to 500 min are:
O—C₂-C₁₂-acylated derivatives of tert-C₄-C₁₂-alkyl hydroperoxides and tert-(C₉-C₁₂-aralkyl) hydroperoxides, such as tert-butyl peroxyacetate, tert-butyl monoperoxymaleate, tert-butyl peroxyisobutyrate, tert-butyl peroxypivalate, tert-butyl peroxyneoheptanoate, tert-butyl peroxy-2-ethylhexanoate, tert-butyl peroxy-3,5,5-trimethylhexanoate, tert-butyl peroxyneodecanoate, tert-amyl peroxypivalate, tert-amyl peroxy-2-ethylhexanoate, tert-amyl peroxyneodecanoate, 1,1,3,3-tetramethylbutyl peroxyneodecanoate, cumyl peroxyneodecanoate, tert-butyl peroxybenzoate, tert-amyl peroxybenzoate and di-tert-butyl diperoxyphthalate;
di-O—C₄-C₁₂-acylated derivatives of tert-C₈-C₁₄-alkylene bisperoxides, such as 2,5-dimethyl-2,5-di(2-ethylhexanoylperoxy)hexane, 2,5-dimethyl-2,5-di(benzoylperoxy)hexane and 1,3-di(2-neodecanoylperoxyisopropyl)benzene;
di(C₂-C₁₂-alkanoyl) and dibenzoyl peroxides, such as diacetyl peroxide, dipropionyl peroxide, disuccinyl peroxide, dicapryloyl peroxide, di(3,5,5-trimethylhexanoyl) peroxide, didecanoyl peroxide, dilauroyl peroxide, dibenzoyl peroxide, di(4-methylbenzoyl) peroxide, di(4-chlorobenzoyl) peroxide and di(2,4-dichlorobenzoyl) peroxide;
tert-C₄-C₅-alkyl peroxy(C₄-C₁₂-alkyl)carbonates, such as tert-amyl peroxy(2-ethyl-hexyl)carbonate;
di(C₂-C₁₂-alkyl) peroxydicarbonates, such as di(n-butyl) peroxydicarbonate and di(2-ethylhexyl) peroxydicarbonate.

Depending on the mean polymerization temperature, examples of particularly suitable initiators (C) are:
at a mean polymerization temperature of from 50 to 60° C.: tert-butyl peroxyneoheptanoate, tert-butyl peroxyneodecanoate, tert-amyl peroxypivalate, tert-amyl peroxyneodecanoate, 1,1,3,3-tetramethylbutyl peroxyneodecanoate, cumyl peroxyneodecanoate, 1,3-di(2-neodecanoyl peroxyisopropyl)benzene, di(n-butyl) peroxydicarbonate and di(2-ethylhexyl) peroxydicarbonate;
at a mean polymerization temperature of from 60 to 70° C.: tert-butyl peroxypivalate, tert-butyl peroxyneoheptanoate, tert-butyl peroxyneodecanoate, tert-amyl peroxypivalate and di(2,4-dichlorobenzoyl) peroxide;
at a mean polymerization temperature of from 70 to 80° C.: tert-butyl peroxypivalate, tert-butyl peroxyneoheptanoate, tert-amyl peroxypivalate, dipropionyl peroxide, dicapryloyl peroxide, didecanoyl peroxide, dilauroyl peroxide, di(2,4-dichlorobenzoyl) peroxide and 2,5-dimethyl-2,5-di(2-ethylhexanoylperoxy)hexane;
at a mean polymerization temperature of from 80 to 90° C.: tert-butyl peroxyisobutyrate, tert-butyl peroxy-2-ethylhexanoate, tert-amyl peroxy-2-ethylhexanoate, dipropionyl peroxide, dicapryloyl peroxide, didecanoyl peroxide, dilauroyl peroxide, di(3,5,5-trimethylhexanoyl) peroxide, dibenzoyl peroxide and di(4-methylbenzoyl) peroxide;
at a mean polymerization temperature of from 90 to 100° C.: tert-butyl peroxyisobutyrate, tert-butyl peroxy-2-ethylhexanoate, tert-butyl monoperoxymaleate, tert-amyl peroxy-2-ethylhexanoate, dibenzoyl peroxide and di(4-methylbenzoyl) peroxide;
at a mean polymerization temperature of from 100 to 110° C.: tert-butyl monoperoxymaleate, tert-butyl peroxyisobutyrate and tert-amyl peroxy(2-ethylhexyl)carbonate;
at a mean polymerization temperature of from 110 to 120° C.: tert-butyl monoperoxymaleate, tert-butyl peroxy-3,5,5-trimethylhexanoate and tert-amyl peroxy(2-ethylhexyl)carbonate.

Preferred initiators (C) are O—C₄-C₁₂-acylated derivatives of tert-C₄-C₅-alkyl hydroperoxides, particular preference being given to tert-butyl peroxypivalate and tert-butyl peroxy-2-ethylhexanoate.

Particularly advantageous polymerization conditions can be established effortlessly by precise adjustment of initiator (C) and polymerization temperature. For instance, the preferred mean polymerization temperature in the case of use of tert-butyl peroxypivalate is from 60 to 80° C., and, in the case of tert-butyl peroxy-2-ethylhexanoate, from 80 to 100° C.

The inventive polymerization reaction can be carried out in the presence of, preferably small, amounts of an organic solvent (D). It is of course also possible to use mixtures of different solvents (D). Preference is given to using water-soluble or water-miscible solvents.

When a solvent (D) is used as a diluent, generally from 1 to 40% by weight, preferably from 1 to 35% by weight, more preferably from 1.5 to 30% by weight, most preferably from 2 to 25% by weight, based in each case on the sum of the components (A), (B1), optionally (B2), and (C), are used.

Examples of suitable solvents (D) include:
monohydric alcohols, preferably aliphatic C₁-C₁₆-alcohols, more preferably aliphatic C₂-C₁₂-alcohols, most preferably C₂-C₄-alcohols, such as ethanol, propanol, isopropanol, butanol, sec-butanol and tert-butanol;
polyhydric alcohols, preferably C₂-C₁₀-diols, more preferably C₂-C₆-diols, most preferably C₂-C₄-alkylene glycols, such as ethylene glycol, 1,2-propylene glycol and 1,3-propylene glycol;
alkylene glycol ethers, preferably alkylene glycol mono(C₁-C₁₂-alkyl) ethers and alkylene glycol di(C₁-C₆-alkyl) ethers, more preferably alkylene glycol mono- and di(C₁-C₂-alkyl) ethers, most preferably alkylene glycol mono(C₁-C₂-alkyl) ethers, such as ethylene glycol monomethyl and -ethyl ether and propylene glycol monomethyl and -ethyl ether;
polyalkylene glycols, preferably poly(C₂-C₄-alkylene) glycols having 2-20 C₂-C₄-alkylene glycol units, more preferably polyethylene glycols having 2-20 ethylene glycol units and polypropylene glycols having 2-10 propylene glycol units, most preferably polyethylene glycols having 2-15 ethylene glycol units and polypropylene glycols having 2-4 propylene glycol units, such as diethylene glycol, triethylene glycol, dipropylene glycol and tripropylene glycol;
polyalkylene glycol monoethers, preferably poly(C₂-C₄-alkylene) glycol mono(C₁-C₂₅-alkyl) ethers having 2-20 alkylene glycol units, more preferably poly(C₂-C₄-alkylene) glycol mono(C₁-C₂₀-alkyl) ethers having 2-20 alkylene glycol units, most preferably poly(C₂-C₃-alkylene) glycol mono(C₁-C₁₆-alkyl) ethers having 3-20 alkylene glycol units;
carboxylic esters, preferably C₁-C₈-alkyl esters of C₁-C₆-carboxylic acids, more preferably C₁-C₄-alkyl esters of C₁-C₃-carboxylic acids, most preferably C₂-C₄-alkyl esters of C₂-C₃-carboxylic acids, such as ethyl acetate and ethyl propionate;
aliphatic ketones which preferably have from 3 to 10 carbon atoms, such as acetone, methyl ethyl ketone, diethyl ketone and cyclohexanone;
cyclic ethers, in particular tetrahydrofuran and dioxane.

The solvents (D) are advantageously those solvents, which are also used to formulate the inventive graft polymers for use (for example in washing and cleaning compositions) and can therefore remain in the polymerization product.

Preferred examples of these solvents are polyethylene glycols having 2-15 ethylene glycol units, polypropylene glycols having 2-6 propylene glycol units and in particular alkoxylation products of C₆-C₈-alcohols (alkylene glycol monoalkyl ethers and polyalkylene glycol monoalkyl ethers).

Particular preference is given here to alkoxylation products of C₈-C₁₆-alcohols with a high degree of branching, which allow the formulation of polymer mixtures which are free-flowing at 40-70° C. and have a very low polymer content at comparatively low viscosity. The branching may be present in the alkyl chain of the alcohol and/or in the polyalkoxylate moiety (copolymerization of at least one propylene oxide, butylene oxide or isobutylene oxide unit). Particularly suitable examples of these alkoxylation products are 2-ethylhexanol or 2-propylheptanol alkoxylated with 1-15 mol of ethylene oxide, C₁₃/C₁₅ oxo alcohol or C₁₂/C₁₄ or C₁₆/C₁₈ fatty alcohol alkoxylated with 1-15 mol of ethylene oxide and 1-3 mol of propylene oxide, preference being given to 2-propylheptanol alkoxylated with 1-15 mol of ethylene oxide and 1-3 mol of propylene oxide.

In one embodiment, the solvent (D) used is water only, with the radical initiator being dissolved in small amounts of organic solvents as disclosed hereinafter.

In another embodiment, polymerization is carried out without the use of a solvent (D) but only with the solvents needed for introducing the radical initiator as disclosed hereinafter.

Small amounts of organic solvents may be used, and preferably are used, for introducing for example the radical initiator as well as the graft monomers (B1) and/or (B2) which might be soluble to a reasonable extent only in such organic solvents but not in water. Suitable organic solvents may be isopropanol, ethanol, 1,2-propandiol and/or tripropylene glycol, and/or other suitable alcohols or organic solvents like 1-methoxy-2-propanol which are considerably inexpensive and available for large-scale uses, or solvents like ethyl acetate, methyl ethyl ketone, and the like, with isopropanol, 1,2-propandiol, 1-Methoxy-2-propanol, ethyl acetate and/or tripropylene glycol being preferred co-solvents, with ethyl acetate and tripropylene glycol being even more preferred, preferably only introduced in the reaction as solvents for the radical initiator and/or the graft monomers (B1) and/or (B2) in as low amounts as possible, preferably only for the radical initiator(s).

In such cases of low overall amounts of alcohols or other organic solvents compared to water, such organic solvents may be left in the final polymer, preferably may be left when the overall amount based on total solvents is less than 1, preferably less than 0,5, more preferably less than 0.1 weight percent. For solvents having a boiling point of less than 110° C. at atmospheric pressure, such solvents may be removed partially or essentially complete by thermal or vacuum distillation or stripping with a gas such as steam or nitrogen, preferably stripping with steam made from water, all at ambient or reduced pressure, whereas higher boiling solvents will usually stay in the polymer products obtained. Hence, solvents like 1-methoxy-2-propanol, 1,2-propandiol and tripropylene glycol will stay in the polymer product, and thus their amounts should be minimized as far as possible by using as high as possible concentrations of the radical initiator.

The radical initiator is preferably employed in the form of a concentrated solution in one of the solvents mentioned before. The concentration of course depends on the solubility of the radical initiator. It is preferred, that the concentration is as high as possible to allow to introduce as little as possible of the organic solvent into the polymerization reaction.

In a most preferred embodiment, the solvent (D) used is water only, with the radical initiator being dissolved in small amounts of organic solvents as disclosed hereinafter. In the process according to the invention, PAG-ester backbone (A), graft monomer (B1) and optional (B2), initiator (C) and, if appropriate, solvent (D) are usually heated to the selected mean polymerization temperature in a reactor.

According to the invention, the polymerization is carried out in such a way that an excess of polymer backbone (A) and formed graft polymer (B)) is constantly present in the reactor. The quantitative ratio of polymer to ungrafted monomer and initiator is generally ≥10:1, preferably ≥15:1 and more preferably ≥20:1.

The polymerization process according to the invention can in principle be carried out in various reactor types.

The reactor used is preferably a stirred tank in which the polymer backbone (A), if appropriate together with portions, of generally up to 15% by weight of the particular total amount, of graft monomers (B1) and (B2), initiator (C) and solvent (D), are initially charged fully or partly and heated to the polymerization temperature, and the remaining amounts of (B), (C) and, if appropriate, (D) are metered in, preferably separately. The remaining amounts of (B), (C) and, if appropriate, (D) are metered in preferably over a period of ≥2 h, more preferably of ≥4 h and most preferably of ≥5 h.

In case more than monomer B1 and/or more than one monomer B2 is employed, such monomers may be added either as one or more mixtures of any of the monomers, such as all vinyl esters in one mixture and all monomers B2 in another mixture; such different mixtures may be added—preferably in parallel—within the same or different time frames.

In the case of the particularly preferred, substantially solvent-free process variant, the entire amount of polymer backbone (A) is initially charged as a melt and the graft monomers (B1) and, if appropriate, (B2), and also the initiator (C) present preferably in the form of a from 10 to 50% by weight solution in one of the solvents (D), are metered in, the temperature being controlled such that the selected polymerization temperature, on average during the polymerization, is maintained with a range of especially +/−10° C., in particular +/−5° C.

In a further particularly preferred, low-solvent process variant, the procedure is as described above, except that solvent (D) is metered in during the polymerization in order to limit the viscosity of the reaction mixture. It is also possible to commence with the metered addition of the solvent only at a later time with advanced polymerization, or to add it in portions.

The polymerization can be affected under standard pressure or at reduced or elevated pressure. When the boiling point of the monomers (B1) or (B2) or of any diluent (D) used is exceeded at the selected pressure, the polymerization is carried out with reflux cooling.

A post-polymerization process step may be added after the main polymerization reaction. For that a further amount of initiator (dissolved in the solvent(s)) can be added over a period of 0.5 hour and up to 3 hours, preferably about 1 to 2 hours, more preferably about 1 hour, with the radical initiator and the solvent(s) for the initiator typically—and preferred—being the same as the ones for the main polymerization reaction. Of course, a different radical initiator and/or different solvent(s) may be employed as well.

In between the post-polymerisation and the main polymerization a certain period of time may be waited, where the main polymerization reaction is left to proceed, before the post-polymerisation reaction is started by starting the addition of further radical initiator.

The temperature of the post-polymerisation process step may be the same as in the main polymerization reaction (which is preferred in this invention), or may be increased. In case increased, it may be typically higher by about 5 to 40° C., preferably 10 to 20° C.

For solvents having a boiling point of approximately less than 110° C. at atmospheric pressure, such solvents may be removed partially or essentially complete by thermal or vacuum distillation or stripping with a gas such as steam or nitrogen, preferably stripping with steam made from water, all at ambient or reduced pressure, whereas higher boiling solvents will usually stay in the polymer products obtained. Hence, such high-boiling solvents will typically stay in the polymer product, and thus their amounts should be minimized as far as possible by e.g. using as high as possible concentrations of the radical initiator, longer polymerization times, post-polymerisation reaction step etc.

The graft polymer of this invention may be subjected to a means of concentration or drying. The graft polymer solution obtained may be concentrated by removing part of the solvent(s) to increase the solid polymer concentration. This may be achieved by distillation processes such as thermal or vacuum distillation, which is performed until the desired solid content is achieved. Such process can be combined with the purification step wherein the graft polymer solution obtained is purified by removing part or all of the volatile components such as volatile solvents and/or unreacted, volatile monomers, by removing the desired amount of solvent.

The graft polymer solution may be also after the main and the optional post-polymerization step and the optional purification step concentrated or dried by subjecting the graft polymer solution to a means of drying such as roller-drum drying, spray-drying, vacuum drying or freeze-drying, preferably—mainly for cost-reasons—spray-drying. Such drying process may be also combined with an agglomeration process such as spray-agglomeration or drying in a fluidized-bed dryer.

### Applications of the Graft Polymers Based on PAG-Ester/Polyalkylene Oxide Polymers as Polymer Backbone

In principle the graft polymers of this invention can be employed in any application to replace conventional graft polymers of the same or very similar composition (in terms of relative amounts of polymer backbone and grafted monomers especially when the type and amounts of grafted monomers is similar or comparable or even almost identical to that of the conventional graft polymers based on PEG or PAGs other than PEG). Such applications are for example:

### Cosmetics, Personal Care

Such compositions and formulations include shampoos, lotions, gels, sprays, soap, make-up powder, lipsticks, hairspray.

### Technical Applications

Such compositions and formulations include glues of any kind, non-water and—preferably—water-based liquid formulations or solid formulations, the use as dispersant in dispersions of any kind, such as in oilfield applications, automotive applications, typically where a solid or a liquid is to be dispersed within another liquid or solid.

### Lacquer, Paints and Colorants Formulations

Such compositions and formulations include non-water- and—preferably—water-based lacquer and colourants, paints, finishings.

### Agricultural Formulations

Such compositions and formulations include formulations and compositions containing agrochemical actives within a liquid or solid environment.

### Aroma Chemical-Formulations

Such compositions and formulations include formulations which dissolve or disperse aroma chemicals in liquid or solid compositions, to evenly disperse and/or retain their stability, so as to retain their aroma profile over extended periods of time; encompassed are also compositions that show a release of aroma chemicals over time, such as extended release or retarded release formulations.

Hence, another subject matter of the present invention is the use of the graft polymers in fabric and home care products, in cosmetic and personal care formulations, as crude oil emulsion breaker, in technical applications including in pigment dispersions for ink jet inks, in formulations for electro plating, in cementitious compositions, in agrochemical formulations as e.g. dispersants, crystal growth inhibitor and/or solubilizer, in lacquer and colorants formulations, preferably in agrochemical compositions and cleaning compositions and in fabric and home care products, in particular cleaning compositions for improved oily and fatty stain removal, removal of solid dirt such as clay, prevention of greying of fabric surfaces, and/or anti-scale agents, wherein the cleaning composition is preferably a laundry detergent formulation and/or a dish wash detergent formulation, more preferably a liquid laundry detergent formulation and/or a liquid manual dish wash detergent formulation, or alternatively in particular in an agrochemical composition, for use as dispersants, crystal growth inhibitor and/or solubilizer.

Another subject-matter of the present invention is, therefore, a cleaning composition, fabric and home care product, industrial and institutional cleaning product, cosmetic or personal care product, oil field-formulation such as crude oil emulsion breaker, pigment dispersion for ink jet inks and inks containing the graft polymer, electro plating product, cementitious composition, a lacquer or paint, and dispersant for agrochemical formulations, comprising at least one graft polymer as defined above.

In a preferred embodiment, it is a cleaning composition and/or fabric and home care product and/or industrial and institutional cleaning product, comprising at least one graft polymer as defined above. In particular, it is a cleaning composition for improved oily and fatty stain removal, preferably a laundry detergent formulation and/or a manual dish wash detergent formulation, more preferably a liquid laundry detergent formulation and/or a liquid manual dish wash detergent formulation.

In one embodiment it is also preferred in the present invention that the cleaning composition comprises (besides at least one graft polymer as described above) additionally at least one enzyme, preferably selected from one or more lipases, hydrolases, amylases, proteases, cellulases, hemicellulases, phospholipases, esterases, pectinases, lactases and peroxidases, and combinations of at least two of the foregoing types.

Another subject-matter of the present invention is, therefore, a cleaning composition such as a fabric and home care product and an industrial and institutional (I&I) cleaning product, comprising at least one graft polymer as defined above, and in particular a cleaning composition for removal of oily and fatty stains.

At least one graft polymer as described herein is present in said inventive cleaning compositions at a concentration of 0.1 to 10, preferably from about 0.25% to 5%, more preferably from about 0.5% to about 3%, and most preferably from about 1% to about 3%, in relation to the total weight of such composition; such cleaning composition may—and preferably does—further comprise a from about 1% to about 70% by weight of a surfactant system.

Preferably, such inventive cleaning composition is a fabric and home care product or an industrial and institutional (I&I) cleaning product, preferably a fabric and home care product, more preferably a laundry detergent or manual dish washing detergent, comprising at least one inventive graft polymer, and optionally further comprising at least one surfactant or a surfactant system, providing improved removal, dispersion and/or emulsification of soils and/or modification of treated surfaces and/or whiteness maintenance of treated surfaces.

Even more preferably, the cleaning compositions of the present invention comprising at least one inventive graft polymer, and optionally further comprising at least one surfactant or a surfactant system, are those for primary cleaning (i.e. removal of stains) within laundry and manual dish wash applications, even more specifically, for removal of oily and fatty stains such as those on fabrics and dishware, and may additionally comprise at least one enzyme selected from the list consisting of lipases, hydrolases, amylases, proteases, cellulases, hemicellulases, phospholipases, esterases, pectinases, lactases and peroxidases, and combinations of at least two of the foregoing types of enzymes.

At least one graft polymer as described herein and/or the at least one graft polymer obtained or obtainable by the inventive process as detailed before is present in said inventive compositions and products at a concentration of from about 0.05% to about 20%, preferably 0.05 to 10%, more preferably from about 0.1% to 8%, even more preferably from about 0.2% to about 6%, and further more preferably from about 0.2% to about 4%, and most preferably in amounts of up to 2%, each in weight % in relation to the total weight of such composition or product, and further including all ranges resulting from selecting any of the lower limits and any of the upper limits and all numbers in between those mentioned; such composition or product may—and preferably does—further comprise from about 1% to about 70% by weight of the composition or product of a surfactant system.

Even more preferably, the compositions or products of the present invention as detailed herein before comprising at least one inventive graft polymer as detailed before and/or at least one graft polymer obtained or obtainable by the inventive process as detailed before and in the amounts as specified in the previous paragraph, optionally further comprising at least one surfactant or a surfactant system in amounts from about 1% to about 70% by weight of the composition or product, are those for primary cleaning (i.e. removal of stains) within laundry applications, and may additionally comprise at least one enzyme selected from lipases, hydrolases, amylases, proteases, cellulases, mannanases, hemicellulases, phospholipases, esterases, xylanases, DNases, dispersins, pectinases, oxidoreductases, cutinases, lactases and peroxidases, more preferably at least two of the aforementioned types.

In one embodiment of the present invention, the inventive graft polymer may be used for soil removal of particulate stains and/or oily and fatty stains, and additionally for whiteness maintenance, preferably in laundry care. In another preferred embodiment the inventive graft polymer may be used for reducing the greying of fabric (anti-greying).

In a preferred embodiment, the cleaning composition of the present invention is a liquid or solid laundry detergent composition.

In another preferred embodiment, the cleaning composition of the present invention is a liquid or solid (e.g. powder or tab/unit dose) detergent composition for manual or automatic dish wash, preferably a liquid manual dish wash detergent composition.

In another embodiment, the cleaning composition of the present invention is a hard surface cleaning composition that may be used for cleaning various surfaces such as hard wood, tile, ceramic, plastic, leather, metal, glass.

In another embodiment, the cleaning composition is designed to be used in cosmetic products, personal care and pet care compositions such as shampoo compositions, body wash formulations, liquid or solid soaps.

In one embodiment, the inventive graft polymers may be utilized in cleaning compositions comprising a surfactant system comprising C10-C15 alkyl benzene sulfonates (LAS) as the primary surfactant and one or more additional surfactants selected from non-ionic, cationic, amphoteric, zwitterionic or other anionic surfactants, or mixtures thereof.

In a further embodiment, the inventive graft polymers may be utilized in cleaning compositions, such as laundry detergents of any kind, and the like, comprising C8-C18 linear or branched alkyl ethersulfates with 1-5 ethoxy-units as the primary surfactant and one or more additional surfactants selected from non-ionic, cationic, amphoteric, zwitterionic or other anionic surfactants, or mixtures thereof.

In a further embodiment the inventive graft polymers may be utilized in cleaning compositions, such as laundry detergents of any kind, and the like, comprising C12-C18 alkyl ethoxylate surfactants with 5-10 ethoxy-units as the primary surfactant and one or more additional surfactants selected from anionic, cationic, amphoteric, zwitterionic or other non-ionic surfactants, or mixtures thereof.

In one embodiment of the present invention, the graft polymer is a component of a cleaning composition, such as preferably a laundry or a dish wash formulation, more preferably a liquid laundry or manual dish wash formulation, that each additionally comprise at least one surfactant, preferably at least one anionic surfactant.

In a further embodiment, this invention also encompasses a composition comprising a graft polymer as described herein before, such composition being preferably a detergent composition, such composition further comprising an antimicrobial agent as disclosed hereinafter, preferably selected from the group consisting of 2-phenoxyethanol, more preferably comprising said antimicrobial agent in an amount ranging from 2 ppm to 5% by weight of the composition; even more preferably comprising 0.1 to 2% of phenoxyethanol.

In a further embodiment, this invention also encompasses a method of preserving an aqueous composition against microbial contamination or growth, such composition comprising a graft polymer as described herein before, such composition being preferably a detergent composition, such method comprising adding at least one antimicrobial agent selected from the disclosed antimicrobial agents as disclosed hereinafter, such antimicrobial agent preferably being 2-phenoxyethanol.

In a further embodiment, this invention also encompasses a composition, preferably a cleaning composition, more preferably a liquid laundry detergent composition or a liquid hand dish composition, even more preferably a liquid laundry detergent composition, or a liquid softener composition for use in laundry, such composition comprising a graft polymer and/or a polymer backbone each as described herein before, such composition further comprising 4,4′-dichoro 2-hydroxydiphenylether in a concentration from 0.001 to 3%, preferably 0.002 to 1%, more preferably 0.01 to 0.6%, each by weight of the composition.

In a further embodiment, this invention also encompasses a method of laundering fabric or of cleaning hard surfaces, which method comprises treating a fabric or a hard surface with a cleaning composition, more preferably a liquid laundry detergent composition or a liquid hand dish composition, even more preferably a liquid laundry detergent composition, or a liquid softener composition for use in laundry, such composition comprising a graft polymer and/or a polymer backbone each as described herein before, such composition further comprising 4,4′-dichoro 2-hydroxydiphenylether.

The selection of the additional surfactants and further ingredients in these embodiments may be dependent upon the application and the desired benefit.

Description of Cleaning Compositions, Formulations and their Ingredients

The phrase “cleaning composition” as used herein includes compositions and formulations designed for cleaning soiled material. Such compositions and formulations include those designed for cleaning soiled material or surfaces of any kind.

Compositions for “industrial and institutional cleaning” includes such cleaning compositions being designed for use in industrial and institutional cleaning, such as those for use of cleaning soiled material or surfaces of any kind, such as hard surface cleaners for surfaces of any kind, including tiles, carpets, PVC-surfaces, wooden surfaces, metal surfaces, lacquered surfaces.

“Compositions for Fabric and Home Care” include cleaning compositions including but not limited to laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, dish washing compositions, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation, preferably during the wash cycle of the laundering or dish washing operation.

The cleaning compositions of the invention may be in any form, namely, in the form of a liquid; a solid such as a powder, granules, agglomerate, paste, tablet, pouches, bar, gel; an emulsion; types delivered in dual- or multi-compartment containers; single-phase or multi-phase unit dose; a spray or foam detergent; premoistened wipes (i.e., the cleaning composition in combination with a nonwoven material such as that discussed in U.S. Pat. No. 6,121,165, Mackey, et al.); dry wipes (i.e., the cleaning composition in combination with a nonwoven materials, such as that discussed in U.S. Pat. No. 5,980,931, Fowler, et al.) activated with water by a user or consumer; and other homogeneous, non-homogeneous or single-phase or multiphase cleaning product forms.

The liquid cleaning compositions of the present invention preferably have a viscosity of from 50 to 10000 mPa*s; liquid manual dish wash cleaning compositions (also liquid manual “dish wash compositions”) have a viscosity of preferably from 100 to 10000 mPa*s, more preferably from 200 to 5000 mPa*s and most preferably from 500 to 3000 mPa*s at 20 1/s and 20° C.; liquid laundry cleaning compositions have a viscosity of preferably from 50 to 3000 mPa*s, more preferably from 100 to 1500 mPa*s and most preferably from 200 to 1000 mPa*s at 20 1/s and 20° C.

The liquid cleaning compositions of the present invention may have any suitable pH-value. Preferably the pH of the composition is adjusted to between 4 and 14. More preferably the composition has a pH of from 6 to 13, even more preferably from 6 to 10, most preferably from 7 to 9. The pH of the composition can be adjusted using pH modifying ingredients known in the art and is measured as a 10% product concentration in demineralized water at 25° C. For example, NaOH may be used and the actual weight % of NaOH may be varied and trimmed up to the desired pH such as pH 8.0. In one embodiment of the present invention, a pH >7 is adjusted by using amines, preferably alkanolamines, more preferably triethanolamine.

Cleaning compositions such as fabric and home care products and formulations for industrial and institutional cleaning, more specifically such as laundry and manual dish wash detergents, are known to a person skilled in the art. Any composition etc. known to a person skilled in the art, in connection with the respective use, can be employed within the context of the present invention by including at least one inventive polymer, preferably at least one polymer in amounts suitable for expressing a certain property within such a composition, especially when such a composition is used in its area of use.

One aspect of the present invention is also the use of the inventive polymers as additives for detergent formulations, particularly for liquid detergent formulations, preferably concentrated liquid detergent formulations, or single mono doses for laundry.

The cleaning compositions of the invention may—and preferably do—contain adjunct cleaning additives (also abbreviated herein as “adjuncts”), such adjuncts being preferably in addition to a surfactant system as defined before.

Suitable adjunct cleaning additives include builders, cobuilders, structurants or thickeners, clay soil removal/anti-redeposition agents, polymeric soil release agents, dispersants such as polymeric dispersing agents, polymeric grease cleaning agents, solubilizing agents, chelating agents, enzymes, enzyme stabilizing systems, bleaching compounds, bleaching agents, bleach activators, bleach catalysts, brighteners, malodor control agents, pigments, dyes, opacifiers, hueing agents, dye transfer inhibiting agents, chelating agents, suds boosters, suds suppressors (antifoams), color speckles, silver care, anti-tarnish and/or anti-corrosion agents, alkalinity sources, pH adjusters, pH-buffer agents, hydrotropes, scrubbing particles, antibacterial agents, anti-oxidants, softeners, carriers, processing aids, pro-perfumes, and perfumes.

Liquid cleaning compositions additionally may comprise—and preferably do comprise at least one of—rheology control/modifying agents, emollients, humectants, skin rejuvenating actives, and solvents.

Solid compositions additionally may comprise—and preferably do comprise at least one of—fillers, bleaches, bleach activators and catalytic materials.

Suitable examples of such cleaning adjuncts and levels of use are found in WO 99/05242, U.S. Pat. Nos. 5,576,282, 6,306,812 B1 and 6,326,348 B1.

Those of ordinary skill in the art will understand that a detersive surfactant encompasses any surfactant or mixture of surfactants that provide cleaning, stain removing, or laundering benefit to soiled material.

Hence, the cleaning compositions of the invention such as fabric and home care products, and formulations for industrial and institutional cleaning, more specifically such as laundry and manual dish wash detergents, preferably additionally comprise a surfactant system and, more preferably, also further adjuncts, as the one described above and below in more detail.

The surfactant system may be composed from one surfactant or from a combination of surfactants selected from anionic surfactants, non-ionic surfactants, cationic surfactants, zwitterionic surfactants, amphoteric surfactants, and mixtures thereof. Those of ordinary skill in the art will understand that a surfactant system for detergents encompasses any surfactant or mixture of surfactants that provide cleaning, stain removing, or laundering benefit to soiled material.

The cleaning compositions of the invention preferably comprise a surfactant system in an amount sufficient to provide desired cleaning properties. In some embodiments, the cleaning composition comprises, by weight of the composition, from about 1% to about 70% of a surfactant system. In other embodiments, the liquid cleaning composition comprises, by weight of the composition, from about 2% to about 60% of the surfactant system. In further embodiments, the cleaning composition comprises, by weight of the composition, from about 5% to about 30% of the surfactant system. The surfactant system may comprise a detersive surfactant selected from anionic surfactants, non-ionic surfactants, cationic surfactants, zwitterionic surfactants, amphoteric surfactants, and mixtures thereof.

### (a) Laundry Compositions

In laundry formulations, anionic surfactants contribute usually by far the largest share of surfactants within such formulation. Hence, preferably, the inventive cleaning compositions for use in laundry comprise at least one anionic surfactant and optionally further surfactants selected from any of the surfactants classes described herein, preferably from non-ionic surfactants and/or amphoteric surfactants and/or zwitterionic surfactants and/or cationic surfactants.

Nonlimiting examples of anionic surfactants—which may be employed also in combinations of more than one surfactant—useful herein include C9-C20 linear alkylbenzenesulfonates (LAS), C10-C20 primary, branched chain and random alkyl sulfates (AS); C10-C18 secondary (2,3) alkyl sulfates; C10-C18 alkyl alkoxy sulfates (AExS) wherein x is from 1 to 30; C10-C18 alkyl alkoxy carboxylates comprising 1 to 5 ethoxy units; mid-chain branched alkyl sulfates as discussed in U.S. Pat. Nos. 6,020,303 and 6,060,443; mid-chain branched alkyl alkoxy sulfates as discussed in U.S. Pat. Nos. 6,008,181 and 6,020,303; modified alkylbenzene sulfonate (MLAS) as discussed in WO 99/05243, WO 99/05242 and WO 99/05244; methyl ester sulfonate (MES); and alpha-olefin sulfonate (AOS).

Preferred examples of suitable anionic surfactants are alkali metal and ammonium salts of C₈-C₁₂-alkyl sulfates, of C₁₂-C₁₈-fatty alcohol ether sulfates, of C₁₂-C₁₈-fatty alcohol polyether sulfates, of sulfuric acid half-esters of ethoxylated C₄-C₁₂-alkylphenols (ethoxylation: 3 to 50 mol of ethylene oxide/mol), of C₁₂-C₁₈-alkylsulfonic acids, of C₁₂-C₁₈ sulfo fatty acid alkyl esters, for example of C₁₂ —C₁₈ sulfo fatty acid methyl esters, of C₁₀-C₁₈-alkylarylsulfonic acids, preferably of n-C₁₀-C₁₈-alkylbenzene sulfonic acids, of C₁₀-C₁₈ alkyl alkoxy carboxylates and of soaps such as for example C₈-C₂₄-carboxylic acids. Preference is given to the alkali metal salts of the aforementioned compounds, particularly preferably the sodium salts.

In one embodiment of the present invention, anionic surfactants are selected from n-C₁₀-C₁₈-alkylbenzene sulfonic acids and from fatty alcohol polyether sulfates, which, within the context of the present invention, are in particular sulfuric acid half-esters of ethoxylated C₁₂-C₁₈-alkanols (ethoxylation: 1 to 50 mol of ethylene oxide/mol), preferably of n-C₁₂-C₁₈-alkanols.

In one embodiment of the present invention, also alcohol polyether sulfates derived from branched (i.e. synthetic) C₁₁-C₁₈-alkanols (ethoxylation: 1 to 50 mol of ethylene oxide/mol) may be employed.

Preferably, the alkoxylation group of both types of alkoxylated alkyl sulfates, based on C₁₂-C₁₈-fatty alcohols or based on branched (i.e. synthetic) C₁₁-C₁₈-alcohols, is an ethoxylation group and an average ethoxylation degree of any of the alkoxylated alkyl sulfates is 1 to 5, preferably 1 to 3.

Preferably, the laundry detergent formulation of the present invention comprises from at least 1 wt % to 50 wt %, preferably in the range from greater than or equal to about 2 wt % to equal to or less than about 30 wt %, more preferably in the range from greater than or equal to 3 wt % to less than or equal to 25 wt %, and most preferably in the range from greater than or equal to 5 wt % to less than or equal to 25 wt % of one or more anionic surfactants as described above, based on the particular overall composition, including other components and water and/or solvents.

In a preferred embodiment of the present invention, anionic surfactants are selected from C10-C15 linear alkylbenzenesulfonates, C10-C18 alkylethersulfates with 1-5 ethoxy units and C10-C18 alkylsulfates.

Non-limiting examples of non-Ionic surfactants—which may be employed also in combinations of more than one other surfactant—include: C8-C18 alkyl ethoxylates, such as, NEODOL® non-ionic surfactants from Shell; ethylenoxide/propylenoxide block alkoxylates as PLURONIC® from BASF; C14-C22 mid-chain branched alkyl alkoxylates, BAEx, wherein x is from 1 to 30, as discussed in U.S. Pat. Nos. 6,153,577, 6,020,303 and 6,093,856; alkylpolysaccharides as discussed in U.S. Pat. No. 4,565,647 Llenado, issued Jan. 26, 1986; specifically alkylpolyglycosides as discussed in U.S. Pat. Nos. 4,483,780 and 4,483,779; polyhydroxy fatty acid amides as discussed in U.S. Pat. No. 5,332,528; and ether capped poly(oxyalkylated) alcohol surfactants as discussed in U.S. Pat. No. 6,482,994 and WO 01/42408.

Preferred examples of non-ionic surfactants are in particular alkoxylated alcohols and alkoxylated fatty alcohols, di- and multiblock copolymers of ethylene oxide and propylene oxide and reaction products of sorbitan with ethylene oxide or propylene oxide, furthermore alkylphenol ethoxylates, alkyl glycosides, polyhydroxy fatty acid amides (glucamides). Examples of (additional) amphoteric surfactants are so-called amine oxides.

Preferred examples of alkoxylated alcohols and alkoxylated fatty alcohols are, for example, compounds of the general formula (A)

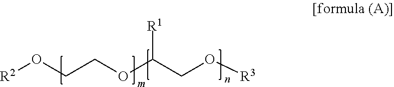

in which the variables are defined as follows:
R1 is selected from linear C1-C10-alkyl, preferably ethyl and particularly preferably methyl,
R2 is selected from C8-C22-alkyl, for example n-C8H17, n-C10H21, n-C12H25, n-C14H29, n-C16H33 or n-C18H37,
R3 is selected from C1-C10-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl,
m and n are in the range from zero to 300, where the sum of n and m is at least one. Preferably, m is in the range from 1 to 100 and n is in the range from 0 to 30.

Here, compounds of the general formula (A) may be block copolymers or random copolymers, preference being given to block copolymers.

Other preferred examples of alkoxylated alcohols and alkoxylated fatty alcohols are, for example, compounds of the general formula (B)

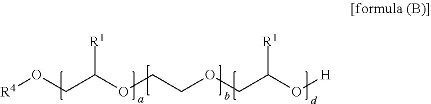

in which the variables are defined as follows:
R¹ is identical or different and selected from linear C1-C4-alkyl, preferably identical in each case and ethyl and particularly preferably methyl,
R⁴ is selected from C6-C20-alkyl, in particular n-C8H₁₇, n-C10H₂₁, n-C12H₂₅, n-C14H₂₉, n-C16H₃₃, n-C18H₃₇,
a is a number in the range from zero to 6, preferably 1 to 6,
b is a number in the range from zero to 20, preferably 4 to 20,
d is a number in the range from 4 to 25.

Preferably, at least one of a and b is greater than zero.

Here, compounds of the general formula (B) may be block copolymers or random copolymers, preference being given to block copolymers.

Further suitable non-ionic surfactants are selected from di- and multiblock copolymers, composed of ethylene oxide and propylene oxide. Further suitable non-ionic surfactants are selected from ethoxylated or propoxylated sorbitan esters. Alkylphenol ethoxylates or alkyl polyglycosides or polyhydroxy fatty acid amides (glucamides) are likewise suitable. An overview of suitable further non-ionic surfactants can be found in EP-A 0 851 023 and in DE-A 198 19 187.

Mixtures of two or more different non-ionic surfactants may of course also be present.

In a preferred embodiment of the present invention, non-ionic surfactants are selected from C12/14 and C16/18 fatty alkoholalkoxylates, C13/15 oxoalkoholalkoxylates, C13-alkoholalkoxylates, and 2-propylheptylalkoholalkoxylates,
each of them with 3-15 ethoxy units, preferably 5-10 ethoxy units, or with 1-3 propoxy- and 2-15 ethoxy units.

Non-limiting examples of amphoteric surfactants—which may be employed also in combinations of more than one other surfactant—include: water-soluble amine oxides containing one alkyl moiety of from about 8 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms. See WO 01/32816, U.S. Pat. Nos. 4,681,704, and 4,133,779. Suitable surfactants include thus so-called amine oxides, such as lauryl dimethyl amine oxide (“lauramine oxide”).

Preferred examples of amphoteric surfactants are amine oxides. Preferred amine oxides are alkyl dimethyl amine oxides or alkyl amido propyl dimethyl amine oxides, more preferably alkyl dimethyl amine oxides and especially coco dimethyl amino oxides. Amine oxides may have a linear or mid-branched alkyl moiety. Typical linear amine oxides include water-soluble amine oxides containing one R1=C8-18 alkyl moiety and two R2 and R3 moieties selected from the group consisting of C1-C3 alkyl groups and C1-C3 hydroxyalkyl groups. Preferably, the amine oxide is characterized by the formula

R1-N(R2)(R3)-O

wherein R1 is a C8-18 alkyl and R2 and R3 are selected from the group consisting of methyl, ethyl, propyl, isopropyl, 2-hydroxethyl, 2-hydroxypropyl and 3-hydroxypropyl. The linear amine oxide surfactants in particular may include linear C10-C18 alkyl dimethyl amine oxides and linear C8-C12 alkoxy ethyl dihydroxy ethyl amine oxides. Preferred amine oxides include linear C10, linear C10-C12, and linear C12-C14 alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the alpha carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2) such that the one alkyl moiety and the one alkyl branch are symmetric. As used herein “symmetric” means that (n1-n2) is less than or equal to 5, preferably 4, most preferably from 0 to 4 carbon atoms in at least 50 wt %, more preferably at least 75 wt % to 100 wt % of the mid-branched amine oxides for use herein. The amine oxide further comprises two moieties, independently selected from a C1-C3 alkyl, a C1-C3 hydroxyalkyl group, or a polyethylene oxide group containing an average of from about 1 to about 3 ethylene oxide groups.

Preferably the two moieties are selected from a C1-C3 alkyl, more preferably both are selected as a C1 alkyl.

In a preferred embodiment of the present invention, amphoteric surfactants are selected from C8-C18 alkyl-dimethyl aminoxides and C8-C18 alkyl-di(hydroxyethyl)aminoxide.

Cleaning compositions may also contain zwitterionic surfactants—which may be employed also in combinations of more than one other surfactant.

Suitable zwitterionic surfactants include betaines, such as alkyl betaines, alkylamidobetaine, amidazoliniumbetaine, sulfobetaine (INCI Sultaines) as well as the phosphobetaines. Examples of suitable betaines and sulfobetaines are the following (designated in accordance with INCI): Almond amidopropyl of betaines, Apricotamidopropyl betaines, Avocadamidopropyl of betaines, Babassuamidopropyl of betaines, Behenamidopropyl betaines, Behenyl of betaines, Canol amidopropyl betaines, Capryl/Capramidopropyl betaines, Camitine, Cetyl of betaines, Cocamidoethyl of betaines, Cocamidopropyl betaines, Cocamidopropyl Hydroxysultaine, Coco betaines, Coco Hydroxysultaine, Coco/Oleam idopropyl betaines, Coco Sultaine, Decyl of betaines, Dihydroxyethyl Oleyl Glycinate, Dihydroxyethyl Soy Glycinate, Dihydroxyethyl Stearyl Glycinate, Dihydroxyethyl Tallow Glycinate, Dimethicone Propyl of PG-betaines, Erucamidopropyl Hydroxysultaine, Hydrogenated Tallow of betaines, Isostearamidopropyl betaines, Lauramidopropyl betaines, Lauryl of betaines, Lauryl Hydroxysultaine, Lauryl Sultaine, Milkamidopropyl betaines, Minkamidopropyl of betaines, Myristamidopropyl betaines, Myristyl of betaines, Oleamidopropyl betaines, Oleamidopropyl Hydroxysultaine, Oleyl of betaines, Olivamidopropyl of betaines, Palmamidopropyl betaines, Palmitamidopropyl betaines, Palmitoyl Camitine, Palm Kemelamidopropyl betaines, Polytetrafluoroethylene Acetoxypropyl of betaines, Ricinoleam idopropyl betaines, Sesamidopropyl betaines, Soyamidopropyl betaines, Stearamidopropyl betaines, Stearyl of betaines, Tallowamidopropyl betaines, Tallowamidopropyl Hydroxysultaine, Tallow of betaines, Tallow Dihydroxyethyl of betaines, Undecylenamidopropyl betaines and Wheat Germamidopropyl betaines.

Preferred betaines are, for example, C₁₂-C₁₈-alkylbetaines and sulfobetaines. The zwitterionic surfactant preferably is a betaine surfactant, more preferable a Cocoamidopropylbetaine surfactant.

Non-limiting examples of cationic surfactants—which may be employed also in combinations of more than one other surfactant—include: the quaternary ammonium surfactants, which can have up to 26 carbon atoms include: alkoxylated quaternary ammonium (AQA) surfactants as discussed in U.S. Pat. No. 6,136,769; dimethyl hydroxyethyl quaternary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Compositions according to the invention may comprise at least one builder. In the context of the present invention, no distinction will be made between builders and such components elsewhere called “co-builders”. Examples of builders are complexing agents, hereinafter also referred to as complexing agents, ion exchange compounds, and precipitating agents. Builders are selected from citrate, phosphates, silicates, carbonates, phosphonates, amino carboxylates and polycarboxylates.

In the context of the present invention, the term citrate includes the mono- and the dialkali metal salts and in particular the mono- and preferably the trisodium salt of citric acid, ammonium or substituted ammonium salts of citric acid as well as citric acid. Citrate can be used as the anhydrous compound or as the hydrate, for example as sodium citrate dihydrate. Quantities of citrate are calculated referring to anhydrous trisodium citrate.

The term phosphate includes sodium metaphosphate, sodium orthophosphate, sodium hydrogenphosphate, sodium pyrophosphate and polyphosphates such as sodium tripolyphosphate. Preferably, however, the composition according to the invention is free from phosphates and polyphosphates, with hydrogenphosphates being subsumed, for example free from trisodium phosphate, pentasodium tripolyphosphate and hexasodium metaphosphate (“phosphate-free”). In connection with phosphates and polyphosphates, “free from” should be understood within the context of the present invention as meaning that the content of phosphate and polyphosphate is in total in the range from 10 ppm to 0.2% by weight of the respective composition, determined by gravimetry.

The term carbonates includes alkali metal carbonates and alkali metal hydrogen carbonates, preferred are the sodium salts. Particularly preferred is Na₂CO₃.

Examples of phosphonates are hydroxyalkanephosphonates and aminoalkanephosphonates. Among the hydroxyalkanephosphonates, the 1-hydroxyethane-1,1-diphosphonate (HEDP) is of particular importance as builder. It is preferably used as sodium salt, the disodium salt being neutral and the tetrasodium salt being alkaline (pH 9). Suitable aminoalkanephosphonates are preferably ethylene diaminetetramethylenephosphonate (EDTMP), diethylenetriaminepentamethylenephosphonate (DTPMP), and also their higher homologues. They are preferably used in the form of the neutrally reacting sodium salts, e.g. as hexasodium salt of EDTMP or as hepta- and octa-sodium salts of DTPMP.

Examples of amino carboxylates and polycarboxylates are nitrilotriacetates, ethylene diamine tetraacetate, diethylene triamine pentaacetate, triethylene tetraamine hexaacetate, propylene diamines tetraacetic acid, ethanol-diglycines, methyiglycine diacetate, and glutamine diacetate. The term amino carboxylates and polycarboxylates also include their respective non-substituted or substituted ammonium salts and the alkali metal salts such as the sodium salts, in particular of the respective fully neutralized compound.

Silicates in the context of the present invention include in particular sodium disilicate and sodium metasilicate, alumosilicates such as for example zeolites and sheet silicates, in particular those of the formula α-Na₂Si₂O₅, β-Na₂Si₂O₅, and δ-Na₂Si₂O₅.

Compositions according to the invention may contain one or more builder selected from materials not being mentioned above. Examples of builders are a-hydroxypropionic acid and oxidized starch.

In one embodiment of the present invention, builder is selected from polycarboxylates. The term “polycarboxylates” includes non-polymeric polycarboxylates such as succinic acid, C₂-C₁₆-alkyl disuccinates, C₂-C₁₆-alkenyl disuccinates, ethylene diamine N,N′-disuccinic acid, tartaric acid diacetate, alkali metal malonates, tartaric acid monoacetate, propanetricarboxylic acid, butanetetracarboxylic acid and cyclopentanetetracarboxylic acid.

Oligomeric or polymeric polycarboxylates are for example polyaspartic acid or in particular alkali metal salts of (meth)acrylic acid homopolymers or (meth)acrylic acid copolymers.

Suitable co-monomers are monoethylenically unsaturated dicarboxylic acids such as maleic acid, fumaric acid, maleic anhydride, itaconic acid and citraconic acid. A suitable polymer is in particular polyacrylic acid, which preferably has a weight-average molecular weight M_{w} in the range from 2000 to 40 000 g/mol, preferably 2000 to 10 000 g/mol, in particular 3000 to 8000 g/mol. Further suitable copolymeric polycarboxylates are in particular those of acrylic acid with methacrylic acid and of acrylic acid or methacrylic acid with maleic acid and/or fumaric acid.

It is also possible to use copolymers of at least one monomer from the group consisting of monoethylenically unsaturated C₃-C₁₀-mono- or C₄-C₁₀-dicarboxylic acids or anhydrides thereof, such as maleic acid, maleic anhydride, acrylic acid, methacrylic acid, fumaric acid, itaconic acid and citraconic acid, with at least one hydrophilically or hydrophobically modified co-monomer as listed below.

Suitable hydrophobic co-monomers are, for example, isobutene, diisobutene, butene, pentene, hexene and styrene, olefins with ten or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having on average 12 to 100 carbon atoms per molecule.

Suitable hydrophilic co-monomers are monomers with sulfonate or phosphonate groups, and also non-ionic monomers with hydroxyl function or alkylene oxide groups. Byway of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols here can comprise 3 to 50, in particular 5 to 40 and especially 10 to 30 alkylene oxide units per molecule.

Particularly preferred sulfonic-acid-group-containing monomers here are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Particularly preferred phosphonate-group-containing monomers are vinylphosphonic acid and its salts.

Moreover, amphoteric polymers can also be used as builders.

Compositions according to the invention can comprise, for example, in the range from in total 0.1 to 70% by weight, preferably 10 to 50% by weight, preferably up to 20% by weight, of builder(s), especially in the case of solid formulations. Liquid formulations according to the invention preferably comprise in the range of from 0.1 to 8% by weight of builder.

Formulations according to the invention can comprise one or more alkali carriers. Alkali carriers ensure, for example, a pH of at least 9 if an alkaline pH is desired. Of suitability are, for example, the alkali metal carbonates, the alkali metal hydrogen carbonates, and alkali metal metasilicates mentioned above, and, additionally, alkali metal hydroxides. A preferred alkali metal is in each case potassium, particular preference being given to sodium. In one embodiment of the present invention, a pH >7 is adjusted by using amines, preferably alkanolamines, more preferably triethanolamine.

In one embodiment of the present invention, the laundry formulation according to the invention comprises additionally at least one enzyme.

Useful enzymes are, for example, one or more hydrolases selected from lipases, amylases, proteases, cellulases, hemicellulases, phospholipases, esterases, pectinases, lactases and peroxidases, and combinations of at least two of the foregoing types.

Such enzyme(s) can be incorporated at levels sufficient to provide an effective amount for cleaning.

The preferred amount is in the range from 0.001% to 5% of active enzyme by weight in the detergent composition according to the invention. Together with enzymes also enzyme stabilizing systems may be used such as for example calcium ions, boric acid, boronic acid, propylene glycol and short chain carboxylic acids. In the context of the present invention, short chain carboxylic acids are selected from monocarboxylic acids with 1 to 3 carbon atoms per molecule and from dicarboxylic acids with 2 to 6 carbon atoms per molecule. Preferred examples are formic acid, acetic acid, propionic acid, oxalic acid, succinic acid, HOOC(CH₂)₃COOH, adipic acid and mixtures from at least two of the foregoing, as well as the respective sodium and potassium salts.

Preferably, the at least one enzyme is a detergent enzyme.

In one embodiment, the enzyme is classified as an oxidoreductase (EC 1), a transferase (EC 2), a hydrolase (EC 3), a lyase (EC 4), an isomerase (EC 5), or a ligase (EC 6). The EC-numbering is according to Enzyme Nomenclature, Recommendations (1992) of the Nomenclature Committee of the International Union of Biochemistry and Molecular Biology including its supplements published 1993-1999. Preferably, the enzyme is a hydrolase (EC 3).

In a preferred embodiment, the enzyme is selected from the group consisting of
proteases, amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the enzyme is selected from the group consisting of proteases, amylases, lipases, cellulases, mannanases, xylanases, DNases, dispersins, pectinases, oxidoreductases, and cutinases, and combinations of at least two of the foregoing types. Most preferably, the enzyme is a protease, preferably, a serine protease, more preferably, a subtilisin protease.

Preferably, the protease is a protease with at least 90% sequence identity to SEQ ID NO: 22 of EP1921147B1 and having the amino acid substitution R101E (according to BPN′ numbering).

Preferably, the amylase is an amylase with at least 90% sequence identity to SEQ ID NO: 54 of WO2021032881A1.

Such enzyme(s) can be incorporated into the composition at levels sufficient to provide an effective amount for achieving a beneficial effect, preferably for primary washing effects and/or secondary washing effects, like anti-greying or antipilling effects (e.g., in case of cellulases). Preferably, the enzyme is present in the composition at levels from about 0.00001% to about 5%, preferably from about 0.00001% to about 2%, more preferably from about 0.0001% to about 1%, or even more preferably from about 0.001% to about 0.5% enzyme protein by weight of the composition.

Preferably, the enzyme-containing composition further comprises an enzyme stabilizing system.

Preferably, the enzyme-containing composition described herein comprises from about 0.001% to about 10%, from about 0.005% to about 8%, or from about 0.01% to about 6%, by weight of the composition, of an enzyme stabilizing system. The enzyme stabilizing system can be any stabilizing system which is compatible with the enzyme.

Preferably, the enzyme stabilizing system comprises at least one compound selected from the group consisting of polyols (preferably, 1,3-propanediol, ethylene glycol, glycerol, 1,2-propanediol, or sorbitol), inorganic salts (preferably, CaCl2, MgCl2, or NaCl), short chain (preferably, C1-C3) carboxylic acids or salts thereof (preferably, formic acid, formate (preferably, sodium formate), acetic acid, acetate, or lactate), borate, boric acid, boronic acids (preferably, 4-formyl phenylboronic acid (4-FPBA)), peptide aldehydes, peptide acetals, and peptide aldehyde hydrosulfite adducts. Preferably, the enzyme stabilizing system comprises a combination of at least two of the compounds selected from the group consisting of salts, polyols, and short chain carboxylic acids and preferably one or more of the compounds selected from the group consisting of borate, boric acid, boronic acids (preferably, 4-formyl phenylboronic acid (4-FPBA)), peptide aldehydes, peptide acetals, and peptide aldehyde hydrosulfite adducts. In particular, if proteases are present in the composition, protease inhibitors may be added, preferably selected from borate, boric acid, boronic acids (preferably, 4-FPBA), peptide aldehydes (preferably, peptide aldehydes like Z-VAL-H or Z-GAY-H), peptide acetals, and peptide aldehyde hydrosulfite adducts.

Compositions according to the invention may comprise one or more bleaching agent (bleaches).

Preferred bleaches are selected from sodium perborate, anhydrous or, for example, as the monohydrate or as the tetrahydrate or so-called dihydrate, sodium percarbonate, anhydrous or, for example, as the monohydrate, and sodium persulfate, where the term “persulfate” in each case includes the salt of the peracid H₂SO₅ and also the peroxodisulfate.

In this connection, the alkali metal salts can in each case also be alkali metal hydrogen carbonate, alkali metal hydrogen perborate and alkali metal hydrogen persulfate. However, the dialkali metal salts are preferred in each case.

Formulations according to the invention can comprise one or more bleach catalysts. Bleach catalysts can be selected from oxaziridinium-based bleach catalysts, bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper- and ruthenium-amine complexes can also be used as bleach catalysts.

Formulations according to the invention can comprise one or more bleach activators, for example tetraacetyl ethylene diamine, tetraacetylmethylene diamine, tetraacetylglycoluril, tetraacetylhexylene diamine, acylated phenolsulfonates such as for example n-nonanoyl- or isononanoyloxybenzene sulfonates, N-methylmorpholinium-acetonitrile salts (“MMA salts”), trimethylammonium acetonitrile salts, N-acylimides such as, for example, N-nonanoylsuccinimide, 1,5-diacetyl-2,2-dioxohexahydro-1,3,5-triazine (“DADHT”) or nitrile quats (trimethylammonium acetonitrile salts).

Formulations according to the invention can comprise one or more corrosion inhibitors. In the present case, this is to be understood as including those compounds which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol or pyrogallol.

In one embodiment of the present invention, formulations according to the invention comprise in total in the range from 0.1 to 1.5% by weight of corrosion inhibitor.

Formulations according to the invention may also comprise further cleaning polymers and/or soil release polymers.

The additional cleaning polymers may include, without limitation, “multifunctional polyethylene imines” (for example BASF's Sokalan® HP20) and/or “multifunctional diamines” (for example BASF's Sokalan® HP96). Such multifunctional polyethylene imines are typically ethoxylated polyethylene imines with a weight-average molecular weight M_{w} in the range from 3000 to 250000, preferably 5000 to 200000, more preferably 8000 to 100000, more preferably 8000 to 50000, more preferably 10000 to 30000, and most preferably 10000 to 20000 g/mol. Suitable multifunctional polyethylene imines have 80 wt % to 99 wt %, preferably 85 wt % to 99 wt %, more preferably 90 wt % to 98 wt %, most preferably 93 wt % to 97 wt % or 94 wt % to 96 wt % ethylene oxide side chains, based on the total weight of the materials. Ethoxylated polyethylene imines are typically based on a polyethylene imine core and a polyethylene oxide shell. Suitable polyethylene imine core molecules are polyethylene imines with a weight-average molecular weight M_{w} in the range of 500 to 5000 g/mol. Preferably employed is a molecular weight from 500 to 1000 g/mol, even more preferred is a M_{w} of 600 to 800 g/mol. The ethoxylated polymer then has on average 5 to 50, preferably 10 to 35 and even more preferably 20 to 35 ethylene oxide (EO) units per NH-functional group.

Suitable multifunctional diamines are typically ethoxylated C2 to C12 alkylene diamines, preferably hexamethylene diamine, which are further quaternized and optionally sulfated. Typical multifunctional diamines have a weight-average molecular weight M_{w} in the range from 2000 to 10000, more preferably 3000 to 8000, and most preferably 4000 to 6000 g/mol. In a preferred embodiment of the invention, ethoxylated hexamethylene diamine, furthermore quaternized and sulfated, may be employed, which contains on average 10 to 50, preferably 15 to 40 and even more preferably 20 to 30 ethylene oxide (EO) groups per NH-functional group, and which preferably bears two cationic ammonium groups and two anionic sulfate groups.

In a preferred embodiment of the present invention, the cleaning compositions may contain at least one multifunctional polyethylene imine and/or at least one multifunctional diamine to improve the cleaning performance, such as preferably improve the stain removal ability, especially the primary detergency of particulate stains on polyester fabrics of laundry detergents. The multifunctional polyethylene imines or multifunctional diamines or mixtures thereof according to the descriptions above may be added to the laundry detergents and cleaning compositions in amounts of generally from 0.05 to 15 wt %, preferably from 0.1 to 10 wt % and more preferably from 0.25 to 5 wt % and even as low as up to 2 wt. %, based on the particular overall composition, including other components and water and/or solvents.

Thus, one aspect of the present invention is a laundry detergent composition, in particular a liquid laundry detergent, comprising (i) at least one inventive polymer and (ii) at least one compound selected from multifunctional polyethylene imines and multifunctional diamines and mixtures thereof.

In one embodiment of the present invention, the ratio of the at least one inventive polymer and (ii) the at least one compound selected from multifunctional polyethylene imines and multifunctional diamines and mixtures thereof, is from 10:1 to 1:10, preferably from 5:1 to 1:5 and more preferably from 3:1 to 1:3.

Laundry formulations comprising the inventive polymer may also comprise at least one antimicrobial agent.

An antimicrobial agent is a chemical compound that kills microorganisms or inhibits their growth or reproduction. Microorganisms can be bacteria, yeasts or molds. A preservative is an antimicrobial agent which may be added to aqueous products and compositions to maintain the original performance, characteristics and integrity of the products and compositions by killing contaminating microorganisms or inhibiting their growth.

The composition/formulation may contain one or more antimicrobial agents and/or preservatives as listed in patent WO2021/115912 A1 (“Formulations comprising a hydrophobically modified polyethyleneimine and one or more enzymes”) on pages 35 to 39.

Especially of interest for the cleaning compositions and fabric and home care products and specifically in the laundry formulations are any of the following antimicrobial agents and/or preservatives:

4,4′-dichloro 2-hydroxydiphenyl ether (further names: 5-chloro-2-(4-chlorophenoxy) phenol, Diclosan, DCPP), Tinosan® HP 100 (30 wt. % of DCPP in in 1,2-propylene glycol); 2-Phenoxyethanol (further names: Phenoxyethanol, Methylphenyiglycol, Phenoxetol, ethylene glycol phenyl ether, Ethylene glycol monophenyl ether, 2-(phenoxy) ethanol, 2-phenoxy-1-ethanol); 2-bromo-2-nitropropane-1,3-diol (further names: 2-bromo-2-nitro-1,3-propanediol, Bronopol); Glutaraldehyde (further names: 1-5-pentandial, pentane-1,5-dial, glutaral, glutardialdehyde); Glyoxal (further names: ethandial, oxylaldehyde, 1,2-ethandial); 5-bromo-5-nitro-1,3-dioxane (further names: 5-bromo-5-nitro-m-dioxane, Bronidox®); Phenoxypropanol (further names: propylene glycol phenyl ether, phenoxyisopropanol 1-phenoxy-2-propanol, 2-phenoxy-1-propanol); Glucoprotamine (chemical description: reaction product of glutamic acid and alkylpropylenediamine, further names: Glucoprotamine 50); Cyclohexyl hydroxyl diazenium-1-oxide, potassium salt (further names: N-cyclohexyl-diazenium dioxide, Potassium HDO, Xyligene,); Formic acid (further names: methanoic acid, Protectol FM, Protectol FM 75, Protectol® FM 85, Protectol® FM 99, Lutensol FM) and its salts, e.g. sodium formiate); Tetrahydro-3,5-dimethyl-1,3,5-thiadia-zine-2-thione (further names: 3,5-dimethyl-1,3-5-thiadiazinane-2-thione, Dazomet; 2,4-dichlorobenzyl alcohol (further names: dichlorobenzyl alcohol, 2,4-dichloro-benzenemethanol, (2,4-dichloro-phenyl)-methanol, DCBA); 1-propanol (further names: n-propanol, propan-1-ol, n-propyl alcohol; 1,3,5-Tris-(2-hydroxyethyl)-hexahydro-1,3,5-triazin (further names: Hexyhydrotriazine, Tris(hydroethyl)-hexyhydrotriazin, hexyhydro-1,3-5-tris(2-hydroxyethyl)-s-triazine, 2,2′,2″-(hexahydro-1,3,5-triazine-1,3,5-triyl)triethanol; 2-butyl-benzo[d]isothiazol-3-one (“BBIT”); 2-methyl-2H-isothiazol-3-one (“MIT”); 2-octyl-2H-isothiazol-3-one (“OIT”); 5-Chloro-2-methyl-2H-isothiazol-3-one (“CIT” or “CMIT”); Mixture of 5-chloro-2-methyl-2H-isothiazol-3-one (“CMIT”) and 2-methyl-2H-isothiazol-3-one (“MIT”) (Mixture of CMIT/MIT); 1,2-benzisothiazol-3(2H)-one (“BIT”); Hexa-2,4-dienoic acid (trivial name “sorbic acid”) and its salts, e.g., calcium sorbate, sodium sorbate; potassium (E,E)-hexa-2,4-dienoate (Potassium Sorbate); Lactic acid and its salts; L-(+)-lactic acid; especially sodium lactate; Benzoic acid and salts of benzoic acid, e.g., sodium benzoate, ammonium benzo-ate, calcium benzoate, magnesium benzoate, MEA-benzoate, potassium benzoate; Salicylic acid and its salts, e.g., calcium salicylate, magnesium salicylate, MEA salicylate, sodium salicylate, potassium salicylate, TEA salicylate; Benzalkonium chloride, benzalkonium bromide, benzalkonium saccharinate; Didecyldimethylammonium chloride (“DDAC”); N-(3-aminopropyl)-N-dodecylpropane-1,3-diamine (“Diamine”); Peracetic acid; Hydrogen peroxide.

At least one antimicrobial agent or preservative may be added to the inventive composition in a concentration of 0.001 to 10% relative to the total weight of the composition.

Preferably, the composition contains 2-phenoxyethanol in a concentration of 0.1 to 2% or 4,4′-dichoro 2-hydroxydiphenyl ether (DCPP) in a concentration of 0.005 to 0.6%.

The invention also encompasses a method of preserving an aqueous composition according to the invention against microbial contamination or growth, which method comprises addition of at least one antimicrobial agent or preservative, preferably 2-phenoxyethanol.

The invention also encompasses a method of providing an antimicrobial effect on textiles after treatment with a solid laundry detergent (e.g. powders, granulates, capsules, tablets, bars etc.), a liquid laundry detergent, a softener or an after-rinse containing 4,4′-dichloro 2-hydroxydiphenyl ether (DCPP).

Formulations according to the invention may also comprise water and/or additional organic solvents, e.g. ethanol or propylene glycol.

Further optional ingredients may be but are not limited to viscosity modifiers, cationic surfactants, foam boosting or foam reducing agents, perfumes, dyes, optical brighteners, and dye transfer inhibiting agents.

### (b) Dish Wash Compositions

Another aspect of the present invention is also a dish wash composition, comprising at least one inventive polymer as described above.

Thus, an aspect of the present invention is also the use of the inventive polymer as described above, in dish wash applications, such as manual or automated dish wash applications.

Dish wash compositions according to the invention can be in the form of a liquid, semi-liquid, cream, lotion, gel, or solid composition, solid embodiments encompassing, for example, powders and tablets.

Liquid compositions are typically preferred for manual dish wash applications, whereas solid formulations and pouch formulations (where the pouches may contain also solids in addition to liquid ingredients) are typically preferred for automated dish washing compositions; however, in some areas of the world also liquid automated dish wash compositions are used and are thus of course also encompassed by the term “dish wash composition”.

The dish wash compositions are intended for direct or indirect application onto dishware and metal and glass surfaces, such as drinking and other glasses, beakers, dish and cooking ware like pots and pans, and cutlery such as forks, spoons, knives and the like.

The inventive method of cleaning dishware, metal and/or glass surfaces comprises the step of applying the dish wash cleaning composition, preferably in liquid form, onto the surface, either directly or by means of a cleaning implement, i.e., in neat form. The composition is applied directly onto the surface to be treated and/or onto a cleaning device or implement such as a dish cloth, a sponge or a dish brush and the like without undergoing major dilution (immediately) prior to the application. The cleaning device or implement is preferably wet before or after the composition is delivered to it. In the method of the invention, the composition can also be applied in diluted form.

Both neat and dilute application give rise to superior cleaning performance, i.e. the formulations of the invention containing at least one inventive polymer exhibit excellent degreasing properties. The effort of removing fat and/or oily soils from the dishware, metal and/or glass surfaces is decreased due to the presence of the inventive polymer, even when the level of surfactant used is lower than in conventional compositions.

Preferably the composition is formulated to provide superior grease cleaning (degreasing) properties, long-lasting suds and/or improved viscosity control at decreased temperature exposures; preferably at least two, more preferably all three properties are present in the inventive dish wash composition. Optional—preferably present—further benefits of the inventive manual dish wash composition include soil removal, shine, and/or hand care; more preferably at least two and most preferably all three further benefits are present in the inventive dish wash composition.

In one embodiment of the present invention, the inventive polymer is one component of a manual dish wash formulation that additionally comprises at least one surfactant, preferably at least one anionic surfactant.

In another embodiment of the present invention, the inventive polymer is one component of a manual dish wash formulation that additionally comprises at least one anionic surfactant and at least one other surfactant, preferably selected from amphoteric surfactants and/or zwitterionic surfactants. In a preferred embodiment of the present invention, the manual dish wash formulations contain at least one amphoteric surfactant, preferably an amine oxide, or at least one zwitterionic surfactant, preferably a betaine, or mixtures thereof, to aid in the foaming, detergency, and/or mildness of the detergent composition.

Examples of suitable anionic surfactants are already mentioned above for laundry compositions. Preferred anionic surfactants for dish wash compositions are selected from C10-C15 linear alkylbenzenesulfonates, C10-C18 alkylethersulfates with 1-5 ethoxy units and C10-C18 alkylsulfates.

Preferably, the manual dish wash detergent formulation of the present invention comprises from at least 1 wt % to 50 wt %, preferably in the range from greater than or equal to about 3 wt % to equal to or less than about 35 wt %, more preferably in the range from greater than or equal to 5 wt % to less than or equal to 30 wt %, and most preferably in the range from greater than or equal to 5 wt % to less than or equal to 20 wt % of one or more anionic surfactants as described above, based on the particular overall composition, including other components and water and/or solvents.

Dish wash compositions according to the invention may comprise at least one amphoteric surfactant.

Examples of suitable amphoteric surfactants for dish wash compositions are already mentioned above for laundry compositions.

Preferred amphoteric surfactants for dish wash compositions are selected from C8-C18 alkyl-dimethyl aminoxides and C8-C18 alkyl-di(hydroxyethyl)aminoxide.

The manual dish wash detergent composition of the invention preferably comprises from 1 wt % to 15 wt %, preferably from 2 wt % to 12 wt %, more preferably from 3 wt % to 10 wt % of the composition of an amphoteric surfactant, preferably an amine oxide surfactant. Preferably the composition of the invention comprises a mixture of the anionic surfactants and alkyl dimethyl amine oxides in a weight ratio of less than about 10:1, more preferably less than about 8:1, more preferably from about 5:1 to about 2:1.

Addition of the amphoteric surfactant provides good foaming properties in the dish wash composition.

Dish wash compositions according to the invention may comprise at least one zwitterionic surfactant.

Examples of suitable zwitterionic surfactants for dish wash compositions are already mentioned above for laundry compositions.

Preferred zwitterionic surfactants for dish wash compositions are selected from betaine surfactants, more preferable from Cocoamidopropylbetaine surfactants.

In a preferred embodiment of the present invention, the zwitterionic surfactant is Cocamidopropylbetaine.

The manual dish wash detergent composition of the invention optionally comprises from 1 wt % to 15 wt %, preferably from 2 wt % to 12 wt %, more preferably from 3 wt % to 10 wt % of the composition of a zwitterionic surfactant, preferably a betaine surfactant.

Dish wash compositions according to the invention may comprise at least one cationic surfactant.

Examples of suitable cationic surfactants for dish wash compositions are already mentioned above for laundry compositions.

Cationic surfactants, when present in the composition, are present in an effective amount, more preferably from 0.1 wt % to 5 wt %, preferably 0.2 wt % to 2 wt % of the composition.

Dish wash compositions according to the invention may comprise at least one non-ionic surfactant.

Examples of suitable non-ionic surfactants for dish wash compositions are already mentioned above for laundry compositions.

Preferred non-ionic surfactants are the condensation products of Guerbet alcohols with from 2 to 18 moles, preferably 2 to 15, more preferably 5-12 of ethylene oxide per mole of alcohol. Other preferred non-ionic surfactants for use herein include fatty alcohol polyglycol ethers, alkylpolyglucosides and fatty acid glucamides.

The manual hand dish detergent composition of the present invention may comprise from 0.1 wt % to 10 wt %, preferably from 0.3 wt % to 5 wt %, more preferably from 0.4 wt % to 2 wt % of the composition, of a linear or branched C10 alkoxylated non-ionic surfactant having an average degree of alkoxylation of from 2 to 6, preferably from 3 to 5. Preferably, the linear or branched C10 alkoxylated non-ionic surfactant is a branched C10 ethoxylated non-ionic surfactant having an average degree of ethoxylation of from 2 to 6, preferably of from 3 to 5. Preferably, the composition comprises from 60 wt % to 100 wt %, preferably from 80 wt % to 100 wt %, more preferably 100 wt % of the total linear or branched C10 alkoxylated non-ionic surfactant of the branched C10 ethoxylated non-ionic surfactant.

The linear or branched C10 alkoxylated non-ionic surfactant preferably is a 2-propylheptyl ethoxylated non-ionic surfactant having an average degree of ethoxylation of from 3 to 5. A suitable 2-propylheptyl ethoxylated non-ionic surfactant having an average degree of ethoxylation of 4 is Lutensol® XP40, commercially available from BASF SE, Ludwigshafen, Germany. The use of a 2-propylheptyl ethoxylated non-ionic surfactant having an average degree of ethoxylation of from 3 to 5 leads to improved foam levels and long-lasting suds.

Thus, one aspect of the present invention is a manual dish wash detergent composition, in particular a liquid manual dish wash detergent composition, comprising (i) at least one inventive polymer, and (ii) at least one further 2-propylheptyl ethoxylated non-ionic surfactant having an average degree of ethoxylation of from 3 to 5.

Dish wash compositions according to the invention may comprise at least one hydrotrope in an effective amount, to ensure the compatibility of the liquid manual dish wash detergent compositions with water.

Suitable hydrotropes for use herein include anionic hydrotropes, particularly sodium, potassium, and ammonium xylene sulfonate, sodium, potassium and ammonium toluene sulfonate, sodium, potassium, and ammonium cumene sulfonate, and mixtures thereof, and related compounds, as disclosed in U.S. Pat. No. 3,915,903.

The liquid manual dish wash detergent compositions of the present invention typically comprise from 0.1 wt % to 15 wt % of the total liquid detergent composition of a hydrotrope, or mixtures thereof, preferably from 1 wt % to 10 wt %, most preferably from 2 wt % to 5 wt % of the total liquid manual dish wash composition.

Dish wash compositions according to the invention may comprise at least one organic solvent.

Examples of organic solvents are C4-C14 ethers and diethers, glycols, alkoxylated glycols, C6-C16 glycol ethers, alkoxylated aromatic alcohols, aromatic alcohols, aliphatic branched alcohols, alkoxylated aliphatic branched alcohols, alkoxylated linear C1-C5 alcohols, linear C1-C5 alcohols, amines, C8-C14 alkyl and cycloalkyl hydrocarbons and halohydrocarbons, and mixtures thereof.

When present, the liquid dish wash compositions will contain from 0.01 wt % to 20 wt %, preferably from 0.5 wt % to 15 wt %, more preferably from 1 wt % to 10 wt %, most preferably from 1 wt % to 5 wt % of the liquid detergent composition of a solvent. These solvents may be used in conjunction with an 35 aqueous liquid carrier, such as water, or they may be used without any aqueous liquid carrier being present. At higher solvent systems, the absolute values of the viscosity may drop but there is a local maximum point in the viscosity profile.

The dish wash compositions herein may further comprise from 30 wt % to 90 wt % of an aqueous liquid carrier, comprising water, in which the other essential and optional ingredients are dissolved, dispersed or suspended. More preferably the compositions of the present invention comprise from 45 wt % to 85 wt %, even more preferably from 60 wt % to 80 wt % of the aqueous liquid carrier. The aqueous liquid carrier, however, may contain other materials which are liquid, or which dissolve in the liquid carrier, at room temperature (25° C.) and which may also serve some other function besides that of an inert filler.

Dish wash compositions according to the invention may comprise at least one electrolyte.

Suitable electrolytes are preferably selected from inorganic salts, even more preferably selected from monovalent salts, most preferably sodium chloride.

The liquid manual dish wash compositions according to the invention may comprise from 0.1 wt % to 5 wt %, preferably from 0.2 wt % to 2 wt % of the composition of an electrolyte.

Manual dish wash formulations comprising the inventive polymer may also comprise at least one antimicrobial agent.

Examples of suitable antimicrobial agents for dish wash compositions are already mentioned above for laundry compositions.

The antimicrobial agent may be added to the inventive hand dish wash composition in a concentration of 0.0001 wt % to 10 wt % relative to the total weight of composition. Preferably, the formulation contains 2-phenoxyethanol in a concentration of 0.01 wt % to 5 wt %, more preferably 0.1 wt % to 2 wt % and/or 4,4′-dichloro 2-hydroxydiphenyl ether in a concentration of 0.001 wt % to 1 wt %, more preferably 0.002 wt % to 0.6 wt % (in all cases relative to the total weight of the composition).

Further additional ingredients are such as but not limited to conditioning polymers, cleaning polymers, surface modifying polymers, soil flocculating polymers, rheology modifying polymers, enzymes, structurants, builders, chelating agents, cyclic diamines, structurants, emollients, humectants, skin rejuvenating actives, carboxylic acids, scrubbing particles, bleach and bleach activators, perfumes, malodor control agents, pigments, dyes, opacifiers, beads, pearlescent particles, microcapsules, antibacterial agents, pH adjusters including NaOH and alkanolamines such as monoethanolamines and buffering means.

As the polymers of the invention are biodegradable, and especially the cleaning formulations typically have a pH of about 7 or higher, and additionally often contain also enzymes—which are included into such cleaning formulations to degrade biodegradable stuff such as grease, proteins, polysaccharides etc which are present in the stains and dirt which shall be removed by the cleaning compositions—some consideration is needed to be taken to formulate those bio-degradable polymers of the invention. Such formulations suitable are in principle known, and include the formulation in solids—where he enzymes and the polymers can be separated by coatings or adding them in separate particles which are mixed—and liquids and semi-liquids, where the polymers and the enzymes can be separated y formulating them in different compartments, such as different compartments of multi-chamber-pouches or bottles having different chambers, from which the liquids are poured out at the same time in a predefined amount to assure the application of the right amount per individual point of use of each component from each chamber. Such multi-compartment-pouches and bottles etc are known to a person of skill as well.

### (c) General Cleaning Compositions and Formulations

In a preferred embodiment the graft polymer according to the present invention is used in a laundry detergent.

Liquid laundry detergents according to the present invention are composed of:

| | |
|---|---|
| 0.05-20% | of at least one inventive polymer |
| 1-50% | of surfactants |
| 0.1-40% | of builders, cobuilders and/or chelating agents |
| 0.1-50% | other adjuncts |

water to add up 100%.

Preferred liquid laundry detergents according to the present invention are composed of:

| | |
|---|---|
| 0.2-6% | of at least one inventive polymer |
| 5-40% | of anionic surfactants selected from C10-C15- |
| | LAS and C10-C18 alkyl ethersulfates containing |
| | 1-5 ethoxy-units |
| 1.5-10% | of nonioic surfactants selected from C10-C18-alkyl |
| | ethoxylates containing 3-10 ethoxy-units |
| 2-20% | of soluble organic builders/cobuilders selected |
| | from C10-C18 fatty acids, di- and tricarboxylic acids, |
| | hydroxy-di- and hydroxytricaboxylic acids and |
| | polycarboxylic acids |
| 0.05-5% | of an enzyme system containing at least one enzyme |
| | suitable for detergent use and preferably also an |
| | enzyme stabilizing system |
| 0.5-20% | of mono- or diols selected from ethanol, isopropanol, |
| | ethylenglycol, or propylenglyclol |
| 0.1-20% | other adjuncts |

water to add up to 100%.

Solid laundry detergents (like e.g. powders, granules or tablets) according to the present invention are composed of:

| | |
|---|---|
| 0.05-20% | of at least one inventive polymer |
| 1-50% | of surfactants |
| 0.1-80% | of builders, cobuilders and/or chelating agents |
| 0-50% | fillers |
| 0-40% | bleach actives |
| 0.1-30% | other adjuncts and/or water |

wherein the sum of the ingredients adds up 100%.

Preferred solid laundry detergents according to the present invention are composed of:

| | |
|---|---|
| 0.2-6% | of at least one inventive polymer |
| 5-30% | of anionic surfactants selected from C10-C15- LAS, |
| | C10-C18 alkylsulfates and C10-C18 alkyl ethersulfates |
| | containing 1-5 ethoxy-units |
| 1.5-7.5% | of non-ionic surfactants selected from C10-C18-alkyl |
| | ethoxylates containing 3-10 ethoxy-units |
| 5-50% | of inorganic builders selected from sodium carbonate, |
| | sodiumbicarbonate, zeolites, soluble silicates, sodium |
| | sulfate |
| 0.5-15% | of cobuilders selected from C10-C18 fatty acids, di- |
| | and tricarboxylic acids, hydroxydi- and hydroxytricarboxylic |
| | acids and polycarboxylic acids |
| 0.1-5% | of an enzyme system containing at least one enzyme suitable |
| | for detergent use and preferably also an enzyme stabilizing |
| | system |
| 0.5-20% | of mono- or diols selected from ethanol, isopropanol, |
| | ethylenglycol, or propylenglyclol |
| 0.1-20% | other adjuncts |

water to ad up to 100%

In a preferred embodiment the polymer according to the present invention is used in a manual dish wash detergent.

Liquid manual dish wash detergents according to the present invention are composed of:

| | |
|---|---|
| 0.05-10% | of at least one inventive polymer |
| 1-50% | of surfactants |
| 0.1-50% | of other adjuncts |

water to add up 100%.

Preferred liquid manual dish wash detergents according to the present invention are composed of:

| | |
|---|---|
| 0.2-5% | of at least one inventive polymer |
| 5-40% | of anionic surfactants selected from C10-C15- LAS, |
| | C10-C18 alkyl ethersulfates containing 1-5 ethoxy- |
| | units, and C10-C18 alkylsulfate |
| 2 10% | of Cocamidopropylbetaine |
| 0-10% | of Lauramine oxide |
| 0-2% | of a non-ionic surfactant, preferably a C10-Guerbet |
| | alcohol alkoxylate |
| 0-5% | of an enzyme, preferably Amylase, and preferably also |
| | an enzyme stabilizing system |
| 0.5-20% | of mono- or diols selected from ethanol, isopropanol, |
| | ethylenglycol, or propylenglyclol |
| 0.1-20% | other adjuncts |

water to add up to 100%

The above and below disclosed liquid formulations may comprise 0 to 2% 2-phenoxyethanol, preferably about 1%, in addition to all other mentioned ingredients.

The above and below disclosed liquid formulations may comprise 0-0.2% 4,4′-dichoro 2-hydroxydiphenylethe, preferably about 0.15%, in addition to all other mentioned ingredients. The bleach-free solid laundry compositions may comprise 0-0.2% 4,4′-dichoro 2-hydroxydiphenylethe, preferably about 0.15%, in addition to all other mentioned ingredients.

The above and below disclosed formulations may—in addition to all other mentioned ingredients—comprise one or more enzymes selected from those disclosed herein above, more preferably a protease and/or an amylase, wherein even more preferably the protease is a protease with at least 90% sequence identity to SEQ ID NO: 22 of EP1921147B1 and having the amino acid substitution R101E (according to BPN′ numbering) and wherein the amylase is an amylase with at least 90% sequence identity to SEQ ID NO: 54 of WO2021032881A1, such enzyme(s) preferably being present in the formulations at levels from about 0.00001% to about 5%, preferably from about 0.00001% to about 2%, more preferably from about 0.0001% to about 1%, or even more preferably from about 0.001% to about 0.5% enzyme protein by weight of the composition.

The following tables show general cleaning compositions of certain types, which correspond to typical compositions correlating with typical washing conditions as typically employed in various regions and countries of the world. The at least one inventive polymer may be added to such formulation(s) in suitable amounts as outlined herein.

When the shown composition does not comprise an inventive graft polymer, such composition is a comparative composition. When it comprises an inventive graft polymer, especially in the amounts that are described herein as preferred, more preferred etc ranges, such compositions are considered to fall within the scope of the present invention.

General formula for laundry detergent compositions
according to the invention:

| | Ranges of Ingredient in Liquid |
|---|---|
| Ingredient | frame formulations |
| Linear alkyl benzene sulphonic acid | 0 to 30% |
| Coco fatty acid | 1 to 12% |
| Fatty alcohol ether sulphate | 0 to 25% |
| NaOH or mono or triethanol amine | Up to pH 7.5 to 9.0 |
| Alcohol ethoxylate | 3 to 10% |
| 1,2-Propylene glycol | 1 to 10% |
| Ethanol | 0 to 4% |
| Sodium citrate | 0 to 8% |
| water | Up to 100% |

Liquid laundry frame formulations according to the invention:

| active | | | | | | |
|---|---|---|---|---|---|---|
| (numbers: % active) | F1 | F2 | F3 | F4 | F5 | F6 |
| alcohol ethoxylat 7EO | 5.40 | 10.80 | 12.40 | 7.30 | 1.60 | 7.60 |
| Coco fatty acid K12-18 | 2.40 | 3.10 | 3.20 | 3.20 | 3.50 | 6.40 |
| Fatty alcohol ether | 5.40 | 8.80 | 7.10 | 7.10 | 5.40 | 14.00 |
| sulphate | | | | | | |
| Linear alkyl benzene | 5.50 | 0.00 | 14.50 | 15.50 | 10.70 | 0.00 |
| sulphonic acid | | | | | | |
| 1,2 Propandiol | 6.00 | 3.50 | 8.70 | 8.70 | 1.10 | 7.80 |
| Triethanolamine | 0 | 0 | 0 | 0 | 0 | 0 |
| Monoethanolamine | 0 | 0 | 4.00 | 4.30 | 0.30 | 0 |
| NaOH | 2.20 | 1.10 | 0 | 0 | 0 | 1.00 |
| Glycerol | 0 | 0.80 | 3.00 | 2.80 | 0 | 0 |
| Ethanol | 2.00 | 0 | 0 | 0 | 0.38 | 0.39 |
| Na citrate | 3.00 | 2.80 | 3.40 | 2.10 | 7.40 | 5.40 |
| Inventive Polymer (s) | 0-5 | 0-5 | 0-5 | 0-5 | 0-5 | 0-5 |
| (total) | | | | | | |
| Protease | 0-1 | 0-1 | 0-1 | 0-1 | 0-1 | 0-1 |
| Amylase | 0-0.5 | 0-0.5 | 0-0.5 | 0-0.5 | 0-0.5 | 0-0.5 |
| Cellulase | 0-0.3 | 0-0.3 | 0-0.3 | 0-0.3 | 0-0.3 | 0-0.3 |
| Lipase | 0-0.2 | 0-0.2 | 0-0.2 | 0-0.2 | 0-0.2 | 0-0.2 |
| Mannanase | 0-0.2 | 0-0.2 | 0-0.2 | 0-0.2 | 0-0.2 | 0-0.2 |
| Pectat Lyase | 0-0.3 | 0-0.3 | 0-0.3 | 0-0.3 | 0-0.3 | 0-0.3 |
| water | to 100 | to 100 | to 100 | to 100 | to 100 | to 100 |

| active | | | | | | | | |
|---|---|---|---|---|---|---|---|---|
| (numbers: % active) | F7 | F8 | F9 | F10 | F11 | F12 | F13 | F14 |
| alcohol ethoxylat 7EO | 3.80 | 0.30 | 13.30 | 8.00 | 5.70 | 20.00 | 9.20 | 29.00 |
| Coco fatty acid K12-18 | 2.80 | 3.00 | 1.70 | 1.80 | 2.50 | 5.00 | 8.60 | 10.40 |
| Fatty alcohol ether | 2.80 | 4.50 | 3.90 | 4.10 | 0 | 10.00 | 22.20 | 0 |
| sulphate | | | | | | | | |
| Linear alkyl benzene | 6.30 | 5.43 | 11.45 | 5.90 | 10.10 | 10.00 | 28.00 | 27.00 |
| sulphonic acid | | | | | | | | |
| 1,2 Propandiol | 0.50 | 0 | 2.50 | 0.40 | 6.00 | 10.00 | 7.00 | 7.00 |
| Triethanolamine | 0 | 0 | 0 | 8.10 | 0 | 0 | 0 | 0 |
| Monoethanolamine | 0.40 | 1.80 | 0 | 0 | 0 | 0 | 8.00 | 7.00 |
| NaOH | 0 | 0 | 2.20 | 0 | 3.30 | 1.50 | 0 | 0 |
| Glycerol | 0 | 0.60 | 0.20 | 1.90 | 0 | 0 | 7.00 | 10.00 |
| Ethanol | 0 | 0 | 1.84 | 0 | 0 | 0 | 0 | 0 |
| Na citrate | 4.60 | 3.30 | 3.30 | 1.40 | 0 | 1.50 | 0 | 0 |
| Inventive Polymer (s) | 0-5 | 0-5 | 0-5 | 0-5 | 0-5 | 0-5 | 0-5 | 0-5 |
| (total) | | | | | | | | |
| Protease | 0-1 | 0-1 | 0-1 | 0-1 | 0-1 | 0-3 | 0-3 | 0-3 |
| Amylase | 0-0.5 | 0-0.5 | 0-0.5 | 0-0.5 | 0-0.5 | 0-0.5 | 0-0.5 | 0-0.5 |
| Cellulase | 0-0.3 | 0-0.3 | 0-0.3 | 0-0.3 | 0-0.3 | 0-0.3 | 0-0.3 | 0-0.3 |
| Lipase | 0-0.2 | 0-0.2 | 0-0.2 | 0-0.2 | 0-0.2 | 0-0.2 | 0-0.2 | 0-0.2 |
| Mannanase | 0-0.2 | 0-0.2 | 0-0.2 | 0-0.2 | 0-0.2 | 0-0.2 | 0-0.2 | 0-0.2 |
| Pectat Lyase | 0-0.3 | 0-0.3 | 0-0.3 | 0-0.3 | 0-0.3 | 0-0.3 | 0-0.3 | 0-0.3 |
| water | to 100 | to 100 | to 100 | to 100 | to 100 | to 100 | to 100 | to 100 |

Laundry powder frame formulations according to the invention:

| | Bleach free Powder | | | | | |
|---|---|---|---|---|---|---|
| Alcohol ethoxylate 7EO | 0.6 | 0 | 1 | 0 | 0 | 5.2 |
| Coco fatty acid K12-18 | 1.2 | 0 | 0 | 0 | 0 | 0 |
| Fatty alcohol ether sulphate | 1.5 | 0 | 0 | 0 | 0 | 6 |
| Linear alkyl benzene sulphonic | 12.1 | 11.2 | 13.6 | 21.9 | 18.7 | 12.7 |
| acid | | | | | | |
| Bleach activator | 0 | 0 | 0 | 0 | 0 | 0 |
| Percarbonate | 0 | 0 | 0 | 0 | 0 | 0 |
| AcetateNa | 0 | 0 | 0 | 0.1 | 0 | 0.1 |
| CitrateNa | 0 | 0 | 0 | 0 | 0 | 14 |
| Na Silicate | 27.9 | 5.8 | 6.6 | 2 | 15 | 20.3 |
| Na Carbonate | 17.2 | 35 | 37.3 | 30.1 | 37 | 1 |
| Na Phospahte | 0 | 0 | 0 | 14 | 0.3 | 0 |
| Na Hydrogencarbonate | 0.7 | 0.9 | 0.5 | 2.7 | 0.4 | 10.5 |
| Zeolite4A | 4.2 | 0.1 | 5.1 | 10.2 | 1.8 | 11.6 |
| HEDP | 0 | 0 | 0 | 0 | 0 | 0.13 |
| MGDA | 0 | 1.1 | 0 | 0 | 0 | 0 |
| Cellulase | 0-0.5 | 0-0.5 | 0-0.5 | 0-0.5 | 0-0.5 | 0-0.5 |
| Lipase | 0-0.4 | 0-0.4 | 0-0.4 | 0-0.4 | 0-0.4 | 0-0.4 |
| Mannanase | 0-0.4 | 0-0.4 | 0-0.4 | 0-0.4 | 0-0.4 | 0-0.4 |
| Protease | 0-1.5 | 0-1.5 | 0-1.5 | 0-1.5 | 0-1.5 | 0-1.5 |
| Amylase | 0-0.5 | 0-0.5 | 0-0.5 | 0-0.5 | 0-0.5 | 0-0.5 |
| Na Sulfate | 30.8 | 1.3 | 33 | 11 | 22 | 3 |
| Na Chloride | 0.2 | 43 | 0.1 | 0 | 0.1 | 0.1 |
| optical brightener | 0.02 | 0 | | 0.1 | 0.06 | |
| Inventive Polymer(s) (total) | 1 | 0 | 0.2 | 2 | 0.5 | 3 |

| | Bleach containing Powder | | | | | |
|---|---|---|---|---|---|---|
| Alcohol ethoxylate 7EO | 1.2 | 5 | 4 | 0.5 | 0.5 | 0 |
| Coco fatty acid K12-18 | 0 | 0 | 0 | 0.3 | 0 | 0.6 |
| Fatty alcohol ether sulphate | 0 | 3.9 | 4.4 | 1.6 | 0 | 0 |
| Linear alkyl benzene | 7.6 | 12.1 | 11.5 | 12.2 | 6.5 | 10.4 |
| sulphonic acid | | | | | | |
| Bleach activator | 0.2 | 9.5 | 9.5 | 0.5 | 0.8 | 2.2 |
| Percarbonate | 3.6 | 19.4 | 16.6 | 2.2 | 11.5 | 5.8 |
| AcetateNa | 0 | 6.7 | 7.1 | 0.3 | 1 | 0.7 |
| CitrateNa | 0 | 1.6 | 8.2 | 0.3 | 0.9 | 1.7 |
| Na Silicate | 3.6 | 11.3 | 16.4 | 10.2 | 9.1 | 16.5 |
| Na Carbonate | 21.6 | 8.7 | 1.4 | 8 | 22.9 | 14.8 |
| Na Phospahte | 0 | 0 | 0 | 0 | 0 | 0 |
| Na Hydrogencarbonate | 0.2 | 2.8 | 1.6 | 0.8 | 0.5 | 0.5 |
| Zeolite4A | 1.6 | 1.4 | 2.4 | 1.6 | 1.8 | 2.3 |
| HEDP | 0 | 0.27 | 0.16 | 0 | 0 | 0.17 |
| MGDA | 0 | 0 | 0 | 0 | 0 | 0 |
| Cellulase | 0-0.5 | 0-0.5 | 0-0.5 | 0-0.5 | 0-0.5 | 0-0.5 |
| Lipase | 0-0.4 | 0-0.4 | 0-0.4 | 0-0.4 | 0-0.4 | 0-0.4 |
| Mannanase | 0-0.4 | 0-0.4 | 0-0.4 | 0-0.4 | 0-0.4 | 0-0.4 |
| Protease | 0-1.5 | 0-1.5 | 0-1.5 | 0-1.5 | 0-1.5 | 0-1.5 |
| Amylase | 0-0.5 | 0-0.5 | 0-0.5 | 0-0.5 | 0-0.5 | 0-0.5 |
| Na Sulfate | 51 | 4 | 6 | 57 | 38 | 37 |
| Na Chloride | 1 | 1 | 0.5 | 1.2 | 0.2 | 1 |
| optical brightener | | 0.29 | 0.1 | 0.23 | 0.13 | 0.19 |
| Inventive Polymer(s) (total) | 2.2 | 9.2 | 2.2 | 0.7 | | 0.4 |

Liquid manual dish wash frame formulations according to the invention:

| Ingredients | MDW.1 | MDW.2 | MDW.3 | MDW.4 | MDW.5 | MDW.6 | MDW.7 | MDW.8 |
|---|---|---|---|---|---|---|---|---|
| Linear C₁₂-C₁₄-alkyl- | 8 | 0 | 6 | 0 | 6 | 0 | 6 | 0 |
| benzenesulfonic acid | | | | | | | | |
| C₁₂-C₁₄-fatty alcohol × | 8 | 16 | 6 | 12 | 6 | 12 | 6 | 12 |
| 2 EO sulfate | | | | | | | | |
| Cocamidopropyl | 0 | 0 | 4 | 4 | 0 | 0 | 2 | 2 |
| betaine | | | | | | | | |
| Lauramine oxide | 0 | 0 | 0 | 0 | 4 | 4 | 2 | 2 |
| 2-Propylheptanol × | 0.5 | 0.5 | 0.5 | 0.5 | 0.5 | 0.5 | 0.5 | 0.5 |
| 4 EO | | | | | | | | |
| Inventive | 1 | 1 | 1 | 1 | 1 | 1 | 1 | 1 |
| Polymer (s) (total) | | | | | | | | |
| Ethanol | 2 | 2 | 2 | 2 | 2 | 2 | 2 | 2 |
| 2-Phenoxyethanol | 1 | 1 | 1 | 1 | 1 | 1 | 1 | 1 |
| (preservative) | | | | | | | | |
| Sodium chloride | 1 | 1 | 1 | 1 | 1 | 1 | 1 | 1 |
| Demin. water | add 100 | add 100 | add 100 | add 100 | add 100 | add 100 | add 100 | add 100 |
| Sodium hydroxide | add pH 8 | add pH 8 | add pH 8 | add pH 8 | add pH 8 | add pH 8 | add pH 8 | add pH 8 |

Further typical liquid detergent formulations LD1, LD2 and LD3 are shown in the following three tables:

Liquid detergent 1- LD1 excellent detergent

Liquid Detergent Formulation

| | |
|---|---|
| Sodium alkylbenzene sulfonic acid (C₁₀-C₁₃) LAS | 9.5% |
| C₁₃/C₁₅ -Oxoalkohol reacted with 7 moles of EO | 4.5% |
| 1,2 propyleneglycol | 6% |
| ethanol | 2% |
| potassium coconut soap | 2.4% |
| NaOH | 2.2% |
| lauryl ether sulphate (Texapon) | 5.0% |
| Sodium citrate | 3% |
| Sokalan HP 20 | 2% |
| Inventive polymer or comparison | 2% |
| Water | to 100% |

Liquid detergent 2- LD2 medium performance detergent

| Liquid Detergent Formulation | |
|---|---|
| Sodium alkylbenzene sulfonic acid (C₁₀-C₁₃) | 5.5% |
| C₁₃/C₁₅ -Oxoalkohol reacted with 7 moles of EO | 5.4% |
| 1,2 propyleneglycol | 6% |
| ethanol | 2% |
| potassium coconut soap | 2.4% |
| Monoethanolamine | 2.5% |
| lauryl ether sulphate | 5.4% |
| Sodium citrate | 3% |
| Sokalan HP96 | 2% |
| Inventive polymer or comparison | 2% |
| Water | to 100% |

Liquid detergent 3- LD3 medium performance biobased detergent

| Liquid Detergent Formulation | |
|---|---|
| MGDA | 5.5% |
| APG, branched C13 Glucosid | 3.5% |
| 1,2 propyleneglycol | 6% |
| ethanol | 2% |
| potassium coconut soap | 4.4% |
| NaOH | 2.2% |
| lauryl ether sulphate | 9.5% |
| Sodium citrate | 3% |
| Inventive polymer or comparison | 2% |
| Water | to 100% |

It is preferred, that within the respective laundry detergent, cleaning composition and/or fabric and home care product, the at least one graft polymer is present in an amount ranging from about 0.01% to about 20%, preferably from about 0.05% to 15%, more preferably from about 0.1% to about 10%, and most preferably from about 0.5% to about 5%, in relation to the total weight of such composition or product.

The specific embodiments as described throughout this disclosure are encompassed by the present invention as part of this invention; the various further options being disclosed in this present specification as “optional”, “preferred”, “more preferred”, “even more preferred” or “most preferred” options of a specific embodiment may be individually and independently (unless such independent selection is not possible by virtue of the nature of that feature or if such independent selection is explicitly excluded) selected and then combined within any of the other embodiments (where other such options and preferences can be also selected individually and independently), with each and any and all such possible combinations being included as part of this invention as individual embodiments.

### Agrochemical Compositions

The invention also relates to an agrochemical composition comprising an agrochemical active ingredient and the graft polymer according to the present invention.

The term “agrochemical active ingredient” refers to a substance that confers a desirable biological activity to the agrochemical formulation. Typically, the agrochemical active ingredient is a pesticide.

Agrochemical active ingredients may be selected from fungicides, insecticides, nematicides, herbicides, safeners, nitrification inhibitors, urease inhibitors, plant growth regulators, micronutrients, biopesticides and/or growth regulators. In one embodiment, the agrochemical active ingredient is an insecticide. In another embodiment, the agrochemical active ingredient is a fungicide. In yet another embodiment the agrochemical active ingredient is a herbicide. The skilled worker is familiar with such pesticides, which can be found, for example, in the Pesticide Manual, 16th Ed. (2013), The British Crop Protection Council, London. Suitable insecticides are insecticides from the class of the carbamates, organophosphates, organochlorine insecticides, phenylpyrazoles, pyrethroids, neonicotinoids, spinosins, avermectins, milbemycins, juvenile hormone analogs, alkyl halides, organotin compounds nereistoxin analogs, benzoylureas, diacylhydrazines, METI acarizides, and insecticides such as chloropicrin, pymetrozin, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or their derivatives. Suitable fungicides are fungicides from the classes of dinitroanilines, allylamines, anilinopyrymidines, antibiotics, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy-(2-amino)pyrimidines, hydroxyanilides, imidazoles, imidazolinones, inorganic substances, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothiolates, phthalamic acids, phthalimides, piperazines, piperidines, propionamides, pyridazinones, pyridines, pyidinylmethylbenzamides, pyrimidinamines, pyrmidines, pyrimidinonehydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyttin compounds, triazines, triazoles. Suitable herbicides are herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyttriazolinones, sulfonylureas, tetrazolinones, thiadiazoles, thiocarbamates, triazines, triazinones, triazoles, triazolinones, triazolocarboxamides, triazolopyrimidines, triketones, uracils, ureas. Suitable plant growth regulators are antiauxins, auxins, cytokinins, defoliants, ethylene modulators, ethylene releasers, gibberellins, growth inhibitors, morphactins, growth retardants, growth stimulators, and further unclassified plant growth regulators. Suitable micronutrients are compounds comprising boron, zinc, iron, copper, manganese, chlorine, and molybdenum. Suitable nitrification inhibitors are linoleic acid, alpha-linolenic acid, methyl p-coumarate, methyl ferulate, methyl 3-(4-hydroxyphenyl) propionate (MHPP), Karanjin, brachialacton, p-benzoquinone sorgoleone, 2-chloro-6-(trichloromethyl)-pyridine (nitrapyrin or N-serve), dicyandiamide (DCD, DIDIN), 3,4-dimethyl pyrazole phosphate (DMPP, ENTEC), 4-amino-1,2,4-triazole hydrochloride (ATC), 1-amido-2-thiourea (ASU), 2-amino-4-chloro-6-methylpyrimidine (AM), 2-mercapto-benzothiazole (MBT), 5-ethoxy-3-trichloromethyl-1,2,4-thiodiazole (terrazole, etridiazole), 2-sulfanilamidothiazole (ST), ammoniumthiosulfate (ATU), 3-methylpyrazol (3-MP), 3,5-dimethylpyrazole (DMP), 1,2,4-triazol thiourea (TU), N-(1H-pyrazolyl-methyl)acetamides such as N-((3(5)-methyl-1H-pyrazole-1-yl)methyl)acetamide, and N-(1H-pyrazolyl-methyl)formamides such as N-((3(5)-methyl-1H-pyrazole-1-yl)methyl formamide, N-(4-chloro-3(5)-methyl-pyrazole-1-ylmethyl)-formamide, N-(3(5), 4-dimethyl-pyrazole-1-ylmethyl)-formamide, neem, products based on ingredients of neem, cyan amide, melamine, zeolite powder, catechol, benzoquinone, sodium terta board, zinc sulfate, 2-(3,4-dimethyl-1H-pyrazol-1-yl)succinic acid (referred to as “DMPSA1” in the following) and/or 2-(4,5-dimethyl-1H-pyrazol-1-yl)succinic acid (referred to as “DMPSA2” in the following), and/or a derivative thereof, and/or a salt thereof; glycolic acid addition salt of 3,4-dimethyl pyrazole (3,4-dimethyl pyrazolium glycolate, referred to as “DMPG” in the following), and/or an isomer thereof, and/or a derivative thereof; citric acid addition salt of 3,4-dimethyl pyrazole (3,4-dimethyl pyrazolium citrate, referred to as “DMPC” in the following), and/or an isomer thereof, and/or a derivative thereof; lactic acid addition salt of 3,4-dimethyl pyrazole (3,4-dimethyl pyrazolium lactate, referred to as “DMPL” in the following), and/or an isomer thereof, and/or a derivative thereof; mandelic acid addition salt of 3,4-dimethyl pyrazole (3,4-dimethyl pyrazolium mandelate, referred to as “DMPM” in the following), and/or an isomer thereof, and/or a derivative thereof; 1,2,4-triazole (referred to as “TZ” in the following), and/or a derivative thereof, and/or a salt thereof; 4-Chloro-3-methylpyrazole (referred to as “CIMP” in the following), and/or an isomer thereof, and/or a derivative thereof, and/or a salt thereof; a reaction adduct of dicyandiamide, urea and formaldehyde, or a triazonyl-formaldehyde-dicyandiamide adduct; 2-cyano-1-((4-oxo-1,3,5-triazinan-1-yl)methyl)guanidine, 1-((2-cyanoguanidino)methyl)urea; 2-cyano-1-((2-cyanoguanidino)methyl)guanidine; 3,4-dimethyl pyrazole phosphate; allyithiourea, and chlorate salts. Examples of envisaged urease inhibitors include N-(n-butyl) thiophosphoric acid triamide (NBPT, Agrotain), N-(n-propyl) thiophosphoric acid triamide (NPPT), 2-nitrophenyl phosphoric triamide (2-NPT), further NXPTs known to the skilled person, phenylphosphorodiamidate (PPD/PPDA), hydroquinone, ammonium thiosulfate, and mixtures of NBPT and NPPT (see e.g. U.S. Pat. No. 8,075,659). Such mixtures of NBPT and NPPT may comprise NBPT in amounts of from 40 to 95% wt.-% and preferably of 60 to 80% wt.-% based on the total amount of active substances. Such mixtures are marketed as LIMUS, which is a composition comprising about 16.9 wt.-% NBPT and about 5.6 wt.-% NPPT and about 77.5 wt.-% of other ingredients including solvents and adjuvants. In one embodiment, the agrochemical active is a fungicide, preferably one or more of following group: Fluxapyroxad, Azoxystrobin, Mefentrifluconazole or Chlorothalonil. In yet another embodiment the agrochemical active is a herbicide, preferably Atrazine.

The agrochemical composition typically comprises a biologically, e.g. a pesticidally effective amount of the agrochemical active ingredient. The term “effective amount” denotes an amount of the composition or of the agrochemical active ingredient, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific agrochemical active ingredient used.

The agrochemical composition typically contains the agrochemical active ingredient in a concentration of from 1 to 70 wt %, preferably from 10 to 50 wt %, more preferably from 20 to 45 wt % based on the total weight of the agrochemical composition. The agrochemical composition typically contains at least 5 wt % of the agrochemical active ingredient, preferably at least 15 wt %, more preferably at least 25 wt %, most preferably at least 35 wt % of the agrochemical active ingredient based on the total weight of the agrochemical composition. The agrochemical composition typically contains up to 95 wt % of the agrochemical active ingredient, preferably up to 65 wt %, more preferably up to least 45 wt % of the agrochemical active ingredient based on the total weight of the agrochemical composition. The active substances are employed in a purity of from 90% to 100%, preferably from 95% to 100% (according to NMR spectrum).

The agrochemical composition contains the graft polymer according to the invention. The concentration of the graft polymer in the agrochemical composition is typically from 0.5 to 20 wt %, preferably from 0.5 to 10 wt %, more preferably from 1 to 8 wt % based on the total weight of the agrochemical composition. The concentration of the graft polymer is typically up to 15 wt %, more preferably up to 9 wt %, most preferably up to 7 wt % based on the total weight of the agrochemical composition. The concentration of the graft polymer is usually at least 2 wt %, preferably at least 2.5 wt % based on the total weight of the agrochemical composition.

The graft polymer according to the invention is typically present in the agrochemical composition in dissolved form, in particular if the agrochemical composition is an aqueous agrochemical composition.

The graft polymer may be present as solid particles, such as dispersed particles, especially if the agrochemical composition is a non-aqueous composition, such as a solid composition or an agrochemical composition with a continuous organic phase.

The weight ration of the active agrochemical ingredient to the graft polymer in the agrochemical composition is typically from 5:1 to 30:1, preferably from 7:1 to 20:1.

The agrochemical composition can be any customary type of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International. Preferred formulation types are suspensions, wettable powders or dusts, and granules, in particular suspensions, and most preferably suspension concentrates.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005. The agrochemical composition is typically prepared by contacting the graft polymer and the active agrochemical ingredient. If the agrochemical composition, the method typically comprises contacting the active agrochemical ingredient with water to form a mill-base. The premix is then typically submitted to grinding or milling to form the final suspension. The graft polymer may either be added to the mill-base or to the final suspension.

In case the agrochemical composition is a granule, it is typically obtained by preparing a premix containing the agrochemical active ingredient, the graft polymer, a filler, and typically up to 5 wt % of water, and the premix is then extruded. The extrudate is then dried and converted to granules.

Suitable auxiliaries that may be added to the agrochemical composition are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, crystal growth inhibitors, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl-naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5. Suitable thickeners are polysaccharides (e.g. xanthan gum, carboxymethylcellulose), anorganic clays (organically modified or unmodified), polycarboxylates, and silicates. Suitable bactericides are bronopol and isothiazolinone derivatives such as alkylisothiazolinones and benzisothiazolinones.

Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants). Suitable tackifiers or binders are polyvinylpyrrolidons, polyvinylacetates, polyvinyl alcohols, polyacrylates, biological or synthetic waxes, and cellulose ethers.

Examples for composition types and their preparation are:
i) Water-soluble concentrates (SL, LS)
   10-60 wt % of an agrochemical active, 5-15 wt % wetting agent (e.g. alcohol alkoxylates), and 1 to 15 wt % of the graft polymer are dissolved in water and/or in a water-soluble solvent (e.g. alcohols) ad 100 wt %. The active substance dissolves upon dilution with water.
ii) Dispersible concentrates (DC)
   5-25 wt % of the agrochemical active ingredient, 1-10 wt % of the graft polymer and optionally further dispersants (e. g. polyvinylpyrrolidone) are dissolved in organic solvent (e.g. cyclohexanone) ad 100 wt %. Dilution with water gives a dispersion.
iii) Emulsifiable concentrates (EC)
   15-70 wt % of an agrochemical active ingredient, 1 to 15 wt % of the graft polymer, 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.
iv) Emulsions (EW, EO, ES)
   5-40 wt % of the agrochemical active ingredient, the 1 to 15 wt % of the graft polymer and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.
v) Suspensions (SC, OD, FS)
   In an agitated ball mill, 20-60 wt % of an agrochemical active ingredient are comminuted with addition of 1-10 wt % the graft polymer and optionally further dispersants, and wetting agents (e.g. sodium lignosulfonate and alcohol ethoxylate), 0,1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.
vi) Water-dispersible granules and water-soluble granules (WG, SG)
   50-80 wt % of the agrochemical active ingredient are ground finely with addition of the graft polymer, optionally further dispersants, and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.
vii) Water-dispersible powders and water-soluble powders (WP, SP, WS)
   50-80 wt % of an agrochemical active ingredient are ground in a rotor-stator mill with addition of 1-5 wt % of the graft polymer and optionally further dispersants (e.g. sodium lignosulfonate), 1-3 wt % wetting agents (e.g. alcohol ethoxylate) and solid carrer (e.g. silica gel) ad 100 wt %. Dilution with water gives a stable dispersion or solution of the active substance.
viii) Gel (GW, GF)
   In an agitated ball mill, 5-25 wt % of an agrochemical active ingredient are comminuted with addition of 3-10 wt % of graft polymer and optionally further dispersants (e.g. sodium lignosulfonate), 1-5 wt % thickener (e.g. carboxymethylcellulose) and water ad 100 wt % to give a fine suspension of the active substance. Dilution with water gives a stable gel of the active substance.
iv) Microemulsion (ME)
   5-20 wt % of an agrochemical active ingredient are added to 5-30 wt % organic solvent blend (e.g. fatty acid dimethylamide and cyclohexanone), 10-25 wt % surfactant blend (e.g. alkohol ethoxylate and arylphenol ethoxylate), 1 to 25 wt % of the graft polymer, and water ad 100%. This mixture is stirred for 1 h to produce spontaneously a thermodynamically stable microemulsion.
iv) Microcapsules (CS)
   An oil phase comprising 5-50 wt % of an agrochemical active ingredient, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of an agrochemical active ingredient, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsules. The monomers amount to 1-10 wt %. The wt % relate to the total CS composition. The microcapsules may then be dispersed in an aqueous composition. To this end, 1 to 40 wt % of the microcapsules are mixed with 2-10 wt % the graft polymer and optionally further dispersants, and wetting agents (e.g. sodium lignosulfonate and alcohol ethoxylate), 0,1-2 wt % thickener (e.g. xanthan gum) and water ad 100 wt % to yield a CS formulation.
ix) Dustable powders (DP, DS)
   1-10 wt % of an agrochemical active ingredient are ground finely and mixed intimately with the 1 to 20 wt % of the graft polymer, and solid carrier (e.g. finely divided kaolin) ad 100 wt %.
x) Granules (GR, FG)
   0.5-30 wt % of an agrochemical active ingredient is ground finely and associated with 1 to 20 wt % of the graft polymer and with solid carrier (e.g. silicate) ad 100 wt %. Granulation is achieved by extrusion, spray-drying or the fluidized bed.
xi) Ultra-low volume liquids (UL)
   1-50 wt % of an agrochemical active ingredient and 1 to 30 wt % of the graft polymer are dissolved in organic solvent (e.g. aromatic hydrocarbon) ad 100 wt %.

The compositions types i) to xi) may optionally comprise further auxiliaries, such as 0,1-1 wt % bactericides, 5-15 wt % anti-freezing agents, 0,1-1 wt % anti-foaming agents, and 0,1-1 wt % colorants.

In one embodiment, the agrochemical composition is a suspension, preferably a suspension concentrate.

The agrochemical suspension typically contains the agrochemical active ingredient in a concentration of from 1 to 65 wt %, preferably from 10 to 60 wt %, more preferably from 20 to 50 wt %, most preferably from 30 to 50 wt % based on the total weight of the agrochemical suspension.

The agrochemical suspension contains at least a portion of the agrochemical active as solid particles suspended in a continuous phase, which is preferably an aqueous continuous phase. Accordingly, the agrochemical suspension is preferably an aqueous agrochemical suspension containing at least 5 wt % of water, preferably at least 10 wt %, more preferably at least 15 wt %, most preferably at least 20 wt %, especially preferably at least 25 wt %, such as at least 30 wt %, in particular at least 40 wt %, each time based on the total weight of the suspension. The agrochemical composition may contain up to 95 wt % of water, preferably up to 80 wt %, more preferably up to 70 wt %, most preferably up to 60 wt % of water, such as up to 50 wt % of water, each time based on the total weight of the suspension.

The agrochemical active ingredient is typically hardly soluble in water. The agrochemical active may have a water-solubility at 20° C. and pH 7 of up to 10 g/I, preferably up to 1 g/l, more preferably up to 0.5 g/l, and most preferably up to 0.1 g/l.

The agrochemical active ingredient is present in the form of suspended particles in the agrochemical suspension. The particles may be characterized by their size distribution, which can be determined by dynamic light scattering techniques. Suitable dynamic light scattering measurement units are inter alia produced under the trade name Malvern Mastersizer 3000. The particles may be characterized by their median diameter, which is usually abbreviated as D50 value. The D50 value refers to a particular particle diameter, wherein half of the particle population by volume is smaller than this diameter. The D50 value is typically determined according to ISO 13320:2009. The particles may have an D50 value of from 0.05 μm to 30 μm, preferably from 0.1 μm to 20 μm, more preferably from 0.5 to 20 μm, most preferably from 0.5 μm to 15 μm, especially preferably from 0.5 μm to 10 μm. The particles typically have an D50 value of at least 0.75 μm, preferably at least 1 μm, and as upper limit preferably not more than 2 μm.

The suspended particles may be present in the form of crystalline or amorphous particles which are solid at 20° C.

Typically, at least 50 wt % of the agrochemical active ingredient may be present as solid particles based on the total weight of the agrochemical active ingredient in the agrochemical suspension, preferably at least 70 wt %, more preferably at least 90 wt %.

The agrochemical suspension may contain a further active ingredient, which may be selected from fungicides, insecticides, nematicides, herbicides, safeners, micronutrients, biopesticides, nitrification inhibitors, urease inhibitors, and/or growth regulators. The further active ingredient may be present in dissolved form or as suspended particles in the agrochemical suspension. The concentration of the further active ingredient is typically from 1 to 50 wt %, preferably from 10 to 25 wt % based on the total weight of the agrochemical suspension.

The agrochemical suspension can in principle be prepared at any pH. Preferably, agrochemical suspensions according to the invention have a pH below 9, more preferably from 4 to 8.

The agrochemical suspension typically contains a thickener. The term “thickener(s)” usually refers to inorganic clays (organically modified or unmodified), such as bentonites, attapulgite, hectorite and smectite clays, and silicates (e.g. colloidal hydrous magnesium silicate, colloidal hydrous aluminium silicate, colloidal hydrous aluminium magnesium silicate, hydrous amorphous silicon dioxide); and organic clays, such as polycarboxylates (e.g. poly(meth)acrylates and modified poly(meth)acrylates), polysaccharides (e.g. xanthan gum, agarose, rhamsan gum, pullulan, tragacanth gum, locust bean gum, guar gum, tara gum, Whelan cum, casein, dextrin, diutan gum, cellulose, ethylcellulose, hydroxyethylcellulose, methylhydroxypropylcellulose), polyvinyl ethers, polyvinyl pyrrolidone, polypropylene oxide—polyethylene ocide condensates, polyvinyl acetates, maleic anhydrides, polypropylene glycols, polyacrylonitrile block copolymers, proteins, and carbohydrates.

The invention also relates to the use of the graft polymer according to the present invention for dispersing agrochemical active ingredients in agrochemical compositions, such as in suspensions.

### Chapter Preferred Agrochemical Composition-Embodiments

The following preferred agrochemical composition-embodiments illustrate the invention to use the graft polymer according to the present invention for dispersing or formulating agrochemical active ingredients in agrochemical compositions and represent, on their own, and in combination, preferred embodiments thereof:

In one embodiment, the agrochemical composition comprises the graft polymer as defined herein and at least one active agrochemical ingredient, preferably any of the preferred actives mentioned herein, more preferably the composition being a suspension, even more preferably the composition containing a continuous aqueous phase, wherein the graft polymer is preferably present in dissolved form.

In a preferred embodiment of the beforementioned embodiment, the composition contains the active agrochemical ingredient in the form of suspended particles.

The composition of any of the beforementioned embodiments in this chapter contains preferably from 1 to 60 wt % of the active agrochemical ingredient based on the total weight of the agrochemical composition, with the active agrochemical ingredient preferably having a water solubility of up to 5 g/l at 20° C., preferably up to 1 g/l at 20° C., and/or contains from 0.5 to 10 wt % of the graft polymer based on the total weight of the agrochemical composition.

In an alternative embodiment, the agrochemical comprises the graft polymer as defined herein and at least one active agrochemical ingredient, preferably any of the preferred actives mentioned herein, more preferably the composition being a suspension, even more preferably the composition containing a continuous aqueous phase, wherein the graft polymer is preferably present at least partially in dissolved form, contains the active agrochemical ingredient in the form of suspended particles.

The composition of the beforementioned embodiment in this chapter contains preferably from 1 to 60 wt % of the active agrochemical ingredient based on the total weight of the agrochemical composition, with the active agrochemical ingredient preferably having a water solubility of up to 5 g/l at 20° C., preferably up to 1 g/l at 20° C., and/or contains from 0.5 to 10 wt % of the graft polymer based on the total weight of the agrochemical composition.

The composition of the two beforementioned embodiments in this chapter, being in the form of a water-dispersible granule or water-dispersible powder.

Further encompassed by this invention is a method for preparing the agrochemical composition according to any of beforementioned embodiments in this chapter, comprising the step of contacting the active agrochemical ingredient with the graft polymer.

Further encompassed by this invention is a method for controlling phytopathogenic fungi and/or undesired plant growth and/or undesired attack by insects or mites and/or for regulating the growth of plants, where the agrochemical composition—being one as defined in any of the beforementioned embodiments in this chapter disclosing such compositions—is allowed to act on the particular pests, their habitat or the plants to be protected from the particular pest, the soil and/or on undesired plants and/or the useful plants and/or their habitat.

Further encompassed by this invention is a method for combating or controlling invertebrate pests, which method comprises contacting said pest or its food supply, habitat or breeding grounds with a pesticidally effective amount of the agrochemical composition, where the agrochemical composition is an agrochemical composition as defined in any of the beforementioned embodiments in this chapter disclosing such compositions.

Further encompassed by this invention is a method for protecting growing plants from attack or infestation by invertebrate pests, which method comprises contacting a plant, or soil or water in which the plant is growing, with a pesticidally effective amount of the agrochemical composition, where the agrochemical composition is an agrochemical composition as defined in any of the beforementioned embodiments in this chapter disclosing such compositions.

Further encompassed is a seed comprising an agrochemical composition in an amount of from 0.1 g to 10 kg per 100 kg of seed, wherein the agrochemical composition is as defined in any of the beforementioned embodiments in this chapter disclosing such compositions.

Further encompassed by this invention is a method for treating or protecting an animal from infestation or infection by invertebrate pests which comprises bringing the animal in contact with a pesticidally effective amount of the agrochemical composition, where the agrochemical composition is an agrochemical composition as defined in any of the beforementioned embodiments in this chapter disclosing such compositions.

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying the agrochemical composition on to plant propagation material, especially seeds include dressing, coating, pelleting, dusting, soaking and in-furrow application methods of the propagation material. Preferably, the agrochemical composition applied on to the plant propagation material by a method such that germination is not induced, e. g. by seed dressing, pelleting, coating and dusting.

The invention also relates to a method for controlling phytopathogenic fungi and/or undesired plant growth and/or undesired attack by insects or mites and/or for regulating the growth of plants, where the agrochemical formulation is allowed to act on the particular pests, their habitat or the plants to be protected from the particular pest, the soil and/or on undesired plants and/or the useful plants and/or their habitat.

In one embodiment, the method is for controlling phytopathogenic fungi. In another embodiment, the method is for controlling undesired vegetation. In another embodiment, the method is for controlling undesired attach by insects or mites.

These methods typically comprise the treatment of the plant to be protected, its locus of growth, the phytopathogenic fungi and/or undesired plant growth and/or undesired attack by insects or mites with the agrochemical composition.

Suitable methods of treatment include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow.

When employed in plant protection, the amounts of agrochemical active applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

Various types of oils, wetters, adjuvants, fertilizer, or micronutrients, and further pesticides (e.g. herbicides, insecticides, fungicides, growth regulators, safeners) may be added to the agrochemical composition as premix or, if appropriate not until immediately prior to use (tank mix). These agents can be admixed with the compositions according to the invention in a weight ratio of 1:100 to 100:1, preferably 1:10 to 10:1.

The user applies the agrochemical composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

The invention also relates to a method for combating or controlling invertebrate pests, which method comprises contacting said pest or its food supply, habitat or breeding grounds with a pesticidally effective amount of the agrochemical composition; to a method for protecting growing plants from attack or infestation by invertebrate pests, which method comprises contacting a plant, or soil or water in which the plant is growing, with a pesticidally effective amount of the agrochemical composition; and to a method for treating or protecting an animal from infestation or infection by invertebrate pests which comprises bringing the animal in contact with a pesticidally effective amount of the agrochemical composition.

Invertebrate pests according to the present invention are typically arachnids, mollusca, or insects, preferably insects.

According to one embodiment, individual components of the composition according to the invention such as parts of a kit or parts of a binary or temary mixture may be mixed by the user himself in a spray tank and further auxiliaries may be added, if appropriate.

In a further embodiment, either individual components of the composition according to the invention or partially premixed components may be mixed by the user in a spray tank and further auxiliaries and additives may be added, if appropriate.

In a further embodiment, either individual components of the composition according to the invention or partially premixed components can be applied jointly (e.g. after tank mix) or consecutively.

The following examples shall further illustrate the present invention without restricting the scope of the invention.

### EXAMPLES

### Polymer Measurements

K-value measures the relative viscosity of dilute polymer solutions and is a relative measure of the average molecular weight. As the average molecular weight of the polymer increases for a particular polymer, the K-value tends to also increase. The K-value is determined in a 3% by weight NaCl solution at 23° C. and a polymer concentration of 1% polymer according to the method of H. Fikentscher in “Cellulosechemie”, 1932, 13, 58.

The number average molecular weight Mn, the weight average molecular weight Mw and the polydispersity Mw/Mn of the polalkylene oxide polymers (the esterified mixtures) for use as polymer backbone A were determined by gel permeation chromatography in tetrahydrofuran. As mobile phase (eluent), tetrahydrofuran comprising 0.035 mol/L diethanolamine was used. The concentration of the esterified polymers in tetrahydrofuran was 2.0 mg per mL. After filtration (pore size 0.2 μm), 100 μL of this solution were injected into a GPC system. Four different columns (heated to 60° C.) were used for separation (SDV precolumn, SDV 1000A, SDV 100 000A, SDV 1 000 000A). The GPC system was operated at a flow rate of 1 mL per min. A DRI Agilent 1100 was used as the detection system. Poly(ethylene glycol) (PEG) standards (PL) having a molecular weight Mn from 106 to 1 378 000 g/mol were used for the calibration.

The number average molecular weight (Mn), the weight average molecular weight (Mw) and the polydispersity Mw/Mn of the inventive graft polymers were determined by gel permeation chromatography in tetrahydrofuran. The mobile phase (eluent) used was tetrahydrofuran comprising 0.035 mol/L diethanolamine. The concentration of graft polymer in tetrahydrofuran was 2.0 mg per mL. After filtration (pore size 0.2 μm), 100 μL of this solution were injected into the GPC system. Four different columns (heated to 60° C.) were used for separation (SDV precolumn, SDV 1000A, SDV 100000A, SDV 1000000A). The GPC system was operated at a flow rate of 1 mL per min. A DRI Agilent 1100 was used as the detection system. Poly(ethylene glycol) (PEG) standards (PL) having a molecular weight Mn from 106 to 1 378 000 g/mol were used for the calibration.

The biodegradation of the polyalkylene oxide polymers for use as polymer backbone A was tested in waste water in triplicate using the OECD 301B manometric respirometry method. 30 mg/mL test substance is inoculated into waste water taken from the waste water treatment plant of Mannheim (Germany) and incubated in a closed flask at 25° C. for 28 days. The consumption of oxygen during this time is measured as the change in pressure inside the flask using an OxiTop C (WTW). Evolved CO2 is absorbed using an NaOH solution. The amount of oxygen consumed by the microbial population during biodegradation of the test substance, after correction using a blank, is expressed as percentage of the theoretical oxygen demand (“ThOD”).

Biodegradation of the graft polymers in waste water was tested in triplicate using the OECD 301F manometric respirometry method. 30 mg/mL test substance is inoculated into wastewater taken from Mannheim Wastewater Treatment Plant and incubated in a closed flask at 25° C. for 28 days. The consumption of oxygen during this time is measured as the change in pressure inside the flask using an OxiTop C (WTV). Evolved CO2 is absorbed using an NaOH solution. The amount of oxygen consumed by the microbial population during biodegradation of the test substance, after correction using a blank, is expressed as a % of the ThOD (Theoretical Oxygen Demand).

### Polyalkylene Oxide Polymers for Use as Polymer Backbone A—Synthesis

### Examples 1 to 10—Oxidation of PAG

In examples 1 to 10, polyalkylene oxides with two primary OH end groups (called “diol”) were oxidized to mixtures containing at least a polyalkylene oxide with two COOH end groups (called “diacid”) and a polyalkylene oxide with one primary OH and one COOH end group (called “monoacid”), and, optionally, also remaining polyalkylene oxide with two primary OH end groups. The mixtures were prepared as follows.

Platinum on charcoal (5.0 wt.-% Pt on C, water content: 59.7 wt.-%, 283 g, 29.2 mmol Pt) was suspended in a mixture of polyalkylene oxide comprising two primary OH end groups (details see table 1) and water (details see table 1), heated to 52° C. and stirred at 800 rpm. Oxygen was passed through the stirred mixture (20 nL/h) via a glass tube, equipped with a glass frit and the temperature was allowed to rise to 60° C. Oxygen dosage and temperature were maintained for the period mentioned in table 1, the oxygen dosage was then stopped and the mixture was allowed to cool down to room temperature. Solids were separated from the liquid phase by filtration and the filter cake was washed with 500 mL of warm water. The washing water was mixed with the filtrate. Water was removed from the liquid mixture by distillation over a wiped film evaporator (overall height: 87.2 cm, diameter 3.54 cm, wiped height: 43 cm, feed: 4.0 mL/min, 44° C., 1.8 kPa abs, 600 rpm). The sump product from the wiped film evaporator was analyzed. The content of OH-groups determined by determination of the hydroxy number, and the content of COOH-groups determined by determination of the acid number. The conversion of the polyalkylene oxides in the partial oxidation was derived from the acid number.

For the partially oxidized mixture based on the low molecular polyalkylene oxide with a Mw value of 200 g/mol, the distribution of the diol, monoacid and diacid was determined by gaschromatography. For this, 0.1 g of a dried sample of the partially oxidized polyalkylene oxide was heated with 1 g of N-methyl-N-(trimethylsilyl)trifiuoracetamide to 80° C. and kept at this temperature for one hour. The resulting mixture was then analyzed via gaschromatography. For the other mixtures based on polyalkylene oxides with 2 400 g/mol, the distribution was calculated from the total content of the OH—and COOH-groups assuming that each OH-group, irrespective of whether being part of the diol or part of the mono-acid, is oxidized with the same probability. The respective values are shown in table 1.

### Examples 11 to 21 and 22—Esterification

Examples 11 to 21 relate to the esterification of the oxidized polyalkylene oxide mixtures obtained by examples 1 to 10 and the determination of the biodegradability of the obtained polyalkylene oxide ester polymers. Example 22 is a comparative example in which the biodegradability of a conventional polyethylene oxide (“PEG”) was determined.

In examples 11 to 21, 98 g of a mixture of polyalkylene oxides obtained by the oxidation procedures described in examples 1 to 10, hereinafter referred to as educt mixture (details see table 2a and 2b), and 2 g water were mixed with an esterification catalyst (details see table 2a and 2b) and heated for a period of time mentioned in table 2a and 2b under vacuum at a pressure of 1 kPa abs, whereby the temperature was slowly increased from 125° C. at the beginning to 145° C. at the end.

The obtained esterified mixture was then analyzed, and the K-value, the number average molar mass Mn and the molecular weight distribution Mw determined as described above. The biodegradability was determined by the OECD 301B degradation test, which is described above.

The average numbers of the ester groups and the ether groups in the polyalkylene oxide ester polymer were estimated from the estimated averaged molecular weights of the respective polyalkylene oxide ester polymers and the respective polyalkylene oxides used in the antecedent oxidation step. For the respective polyalkylene oxide ester polymers, the number average molecular weight Mn was used since it is a good measure of the average molecular weight on a molecular scale. Regarding the respective polyalkylene oxides used in the antecedent oxidation step, the molecular average molecular weight Mw could be alternatively used since the polyethylene oxides typically have a low polydispersity PD slightly above 1, so that Mw and Mn only slightly differ.

The number of the ester groups in the polyalkylene oxide ester polymers, which were based on the use of partially oxidized polyethylene oxides (relating to a ratio of the oxidized OH groups of around 50%), was estimated as follows. First, the number of the structural units was estimated by dividing (1) the number average molecular weight Mn of the polyalkylene oxide ester polymer, which has been corrected by 18 g/mol considering the two end groups, (2) by the average molecular weight of the esterified structural elements. The latter was calculated by the molecular average molecular weight Mw of the used polyethylene oxide minus 18 g/mol, considering that water is split off by the esterification, plus 16 g/mol minus 2 g/mol, considering that in the average arithmetically one —CO—unit per structural element was formed from the respective —CH2- unit. In case of the structural element of formula (I), it is exactly one per structural element, in case of a combination of structural elements of formula (II) and (III), it is two and zero leading also to one in the average. Second, the number of the structural units was reduced by 1 to consider that each ester group in the polyalkylene oxide ester polymer of the examples connects two structural elements, so that there is one structural element more than ester groups.

The above-mentioned estimation is specifically explained for example 12. The number average molecular weight Mn of the polyalkylene oxide ester polymer was 2400 g/mol, leading to a value of 2382 g/mol. The average molecular weight of the esterified structural elements was (400-4) g/mol=396 g/mol. This leads to an average number of structural units of 2382/396=6.0 and consequently to an average number of ester groups of 5.0, or expressed in a mathematical equation

$$\frac{( 2400 - 18 )}{( 400 - 4 )} - 1 = 5. .$$

The number of the ether groups in the polyalkylene oxide ester polymers, which were based on the use of partially oxidized polyethylene oxides (relating to a ratio of the oxidized OH groups of around 50%), was estimated as follows. First, the number of the ethylene oxide units in the used polyethylene oxide was calculated by dividing (1) the molecular average molecular weight Mw of the used polyethylene oxide minus 18 g/mol, considering the end groups formally formed by the addition of one molecule of water per polyethylene oxide molecule during the polymerization, (2) by 44 g/mol which is the molecular weight of a —CH2CH2-O—unit. Second, the number of the ethylene oxide units was reduced by 1 to consider that each polyethylene oxide has one ether group less than the number of the ethylene oxide units. The result is the average number of the ether groups in the polyethylene oxide. Third, this number was then multiplied with the number of the structural units in the polyalkylene oxide ester polymers, which was calculated as described above.

The above-mentioned estimation is specifically explained for example 12. The molecular average molecular weight Mw of the used polyethylene oxide was 400 g/mol leading to a value of 382 g/mol. Its division by 44 g/mol leads to a number of 8.7 —CH2CH2-O— units, which at the end lead to an average of 7.7 ether units in the polyethylene oxide. Since the average number of the structural units in the polyalkylene oxide ester polymer was estimated above as 6.0, the average number of the ether groups in the polyalkylene oxide ester polymer was 46, or expressed in a mathematical equation

$$[ \frac{400 - 18}{44} - 1 ] \times \frac{2400 - 18}{400 - 4} = 46 .$$

In the examples which were based on the use of fully oxidized polyethylene oxides (relating to a ratio of the oxidized OH groups of around 95 to 100%) and therefore required the addition of a diol as second component, the number of the ester groups and ether groups was estimated in a similar way as described above with the main difference that the molecular average molecular weight Mw of the used polyethylene oxides in the calculation was the arithmetic mean value between the molecular average molecular weight Mw of the polyalkylene oxide used in the oxidation and the molecular average molecular weight Mw of the polyalkylene oxide used as diol component. This approach is based on the simplified assumption that both structural units have been equally distributed in the polyalkylene oxide ester polymers.

This modified estimation is specifically explained for example 14, in which polyethylene oxide with Mw=600 g/mol was nearly fully oxidized and polyethylene oxide with Mw=1500 g/mol used as diol component. The mathematical equation for the estimation of the ester groups is

$$\frac{4100 - 18}{[ ( 600 + 1500 ) \times 0.5 ] - 4} - 1 = 2.9 .$$

and for the estimation of the ether groups

$$\{ \frac{[ ( 600 + 1500 ) \times 0.5 ] - 18}{44} - 1 \} \times \frac{4100 - 18}{[ ( 600 + 1500 ) \times 0.5 ] - 4} = 88.$$

All polyalkylene oxide ester polymers obtained in examples 11 to 21 show a biodegradability in the range of 73 to 89% after 28 days, measured as CO2 formation relative to the theoretical value, although the weight average molecular weight Mw covers 4 050 to even 18 300 g/mol. In contrast to that the biodegradability of a conventional polyethylene oxide with Mw=8720 g/mol, measured by its CO2 formation within 28 days, is only very poor with a value of 16% although its Mw value is well below 10 000 g/mol.

In addition to that, the used polyalkylene oxide ester polymers are significantly better biodegradable than the conventional PEG 9000 polymer, leading to a biodegradation degree of high 73 to 79% for the polyalkylene oxide ester polymers and very low 16% for the conventional PEG 9000 polymer.

Further examples 23 to 29 were also tested for biodegradation and are shown in Polymer Backbone-Table 3.

Polymer backbone-Table 1 - Oxidation of PAG Examples 1 to 10

| Example | | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|---|
| Starting material descriptor | | A1 | A2 | A3 | A4 | A5 | A6 | A7 | A8 | A9 | A10 |
| Type ^{#1} and molecular average molecular weight | | EO | EO | EO | EO | EO | EO | EO | EO | EO | EO |
| M_{w} [g/mol] of polyalkylene oxide | | 200 | 400 | 600 | 600 | 1000 | 1000 | 1500 | 1500 | 2000 | 2000 |
| Number average of number of ether groups | | 3-4 | 8 | 12 | 12 | 21 | 21 | 33 | 33 | 44 | 44 |
| Amount of polyalkylene oxide [g] | | 1500 | 1500 | 1500 | 1500 | 1500 | 1500 | 1500 | 1500 | 1500 | 1500 |
| Amount of water [g] | | 3500 | 3500 | 3500 | 3500 | 3500 | 3500 | 3500 | 3500 | 3500 | 3500 |
| Oxidation time [h] | | 6 | 6.5 | 35 | 8.5 | 45 | 10 | 62 | 11 | 85 | 12.5 |
| Ratio in | diacid | 30 | 25 | 94 | 26 | 97 | 25 | 96 | 24 | 98 | 24 |
| product [%] | mono acid | 37 | 50 | 5.8 | 50 | 3.1 | 50 | 3.9 | 50 | 2.0 | 50 |
| | diol ^{#2} | 28 | 25 | 0.1 | 24 | 0.0 | 25 | 0.0 | 26 | 0.0 | 27 |

| | | | | | | | | | | |
|---|---|---|---|---|---|---|---|---|---|---|
| Ratio of oxidized OH groups [%] ^{#2} | 48.8 | 49.7 | 97.0 | 51.5 | 98.4 | 49.8 | 98.0 | 49.4 | 99.0 | 48.5 |
| Physical property | liquid | liquid | liquid | liquid | solid | paste | solid | solid | solid | solid |
| Hydroxy number | 231.0 | 117.1 | <2 | 79.1 | <2 | 48.0 | 2.3 | 32 | <2 | 22.7 |
| [mg KOH/g] | | | | | | | | | | |
| Acid number | 268.3 | 136.8 | 173.8 | 91.7 | 106.2 | 54.4 | 70.2 | 37.1 | 51.8 | 26.0 |
| [mg KOH/g] | | | | | | | | | | |

^{#1} EO = polyethylene oxide/EOPOEO = polypropylene oxide with —CH₂CH₂OH end groups
^{#2} Calculated on basis of the percentages of mono- and diacids

Polymer backbone-Table 2a - Esterification to PAG-Ester Examples 11 to 20

| Example | Ex. 11 | Ex. 12 | Ex. 13 | Ex. 14 | Ex. 15 | Ex. 16 |
|---|---|---|---|---|---|---|
| Polymer backbone | B1 | B2 | B3 | B3.2 | B4 | B5 |
| descriptor | | | | | | |
| Classification ^{#1} | inv. | inv. | inv. | inv. | inv. | inv. |
| Educt descriptor | A1 | A2 | A3 | A3 | A4 | A5 |
| Educt from | Ex. 1 | Ex. 2 | Ex. 3 | Ex. 3 | Ex. 4 | Ex. 5 |
| Esterification catalyst ^{#2,3} | 0.2 wt.-% | 0.3 wt-% | 0.15 wt-% | 0.2 wt-% | 0.1 wt.-% | 0.25 wt-% |
| | cat 1 | cat 2 | cat 3 | cat 3 | cat 1 | cat 2 |
| Further diol ^{#4} | — | — | 550 g | 370 g | — | 234 g |
| | | | PEG 600 | PEG 1500 | | PEG 400 |
| Reaction time [h] | 5.5 | 6 | 8.5 | 10 | 8.0 | 5.0 |
| K-value | 9.9 | 13.6 | 23.3 | 26.6 | 26.7 | 28.6 |
| Physical property | liquid | liquid | liquid | paste | liquid | paste |
| Mₙ [g/mol] | 1580 | 2400 | 3600 | 4100 | 4350 | 3700 |
| M_{w} [g/mol] | 4050 | 5450 | 10 600 | 13 600 | 14 820 | 14 470 |
| PD (=M_{w}/Mₙ) | 2.56 | 2.27 | 2.94 | 3.32 | 3.41 | 3.91 |
| Average number of ether | 25 | 46 | 73 | 88 | 89 | 77 |
| groups in polymer ^{#5} | | | | | | |
| Average number of ester | 7.0 | 5.0 | 5.0 | 2.9 | 6.3 | 4.3 |
| groups in polymer ^{#5} | | | | | | |
| Biodegradation after 28 | 89 | 86.5 | 80.6 | 82.6 | 83.5 | 79 |
| days according to | | | | | | |
| OECD 301B [%] ^{#6} | | | | | | |

| Example | Ex. 17 | Ex. 18 | Ex. 19 | Ex. 20 |
|---|---|---|---|---|
| Polymer backbone | B6 | B7 | B8 | B9 |
| descriptor | | | | |
| Classification ^{#1} | inv. | inv. | inv. | inv. |
| Educt descriptor | A6 | A7 | A8 | A9 |
| Educt from | Ex. 6 | Ex. 7 | Ex. 8 | Ex. 9 |
| Esterification catalyst ^{#2,3} | 0.3 wt-% | 0.2 wt.-% | 0.15 wt.-% | 0.2 wt-% |
| | cat 2 | cat 1 | cat 1 | cat 3 |
| Further diol ^{#4} | — | 565 g | — | 290 g |
| | | PEG 1500 | | PEG 1000 |
| Reaction time [h] | 5.5 | 8 | 5 | 7 |
| K-value | 30.5 | 32 | 29 | 33.5 |
| Physical property | solid | solid | solid | solid |
| Mₙ [g/mol] | 4230 | 5150 | 4940 | 6400 |
| M_{w} [g/mol] | 13 700 | 16 850 | 16 100 | 18 300 |
| PD (=M_{w}/Mₙ) | 3.24 | 3.27 | 3.26 | 2.86 |
| Average number of ether | 90 | 112 | 108 | 139 |
| groups in polymer ^{#5} | | | | |
| Average number of ester | 3.2 | 2.4 | 2.3 | 3.3 |
| groups in polymer ^{#5} | | | | |
| Biodegradation after 28 | 75 | 73 | 76 | 74 |
| days according to | | | | |
| OECD 301B [%] ^{#6} | | | | |

Annotation to Polymer backbone-Table 2a:
^{#1} inv. = inventive/comp. = comparative
^{#2} cat 1 = Ti(IV)-tetraisobutylat/cat 2 = methanesulfonic acid/cat 3 = Zn-octanoate
^{#3} wt.-% relates to the educt mixture plus water.
^{#4} PEG = polyethylene glycol = polyethylene oxide with two primary OH end groups
^{#5} Calculated as described in the description of examples 11 to 22.
^{#6} The percent values relate to the CO₂ formation relative to the theoretical value.

Polymer backbone-Table 2b Examples 21 to 22

| Example | Ex. 21 | Ex. 22 |
|---|---|---|
| Polymer backbone | B10 | — |
| descriptor | | |
| Classification ^{#1} | inv. | comp. |
| Educt descriptor | A10 | |
| Educt mixture from ^{#4} | Ex. 10 | PEG 9000 |
| Esterification catalyst ^{#2,3} | 0.2 wt.-% | — |
| | cat 1 | |
| Further diol ^{#4} | — | — |
| Reaction time [h] | 4.5 | — |
| K-value | 30.4 | 25.4 |
| Physical property | solid | solid |
| Mₙ [g/mol] | 5400 | 6830 |
| M_{w} [g/mol] | 16 630 | 8720 |
| PD (=M_{w}/Mₙ) | 3.08 | 1.28 |
| Average number of ether | 119 | 203 |
| groups in polymer ^{#5} | | |
| Average number of ester | 1.7 | — |
| groups in polymer ^{#5} | | |
| Biodegradation after 28 | 79 | 16 |
| days according to | | |
| OECD 301B [%] ^{#6} | | |

^{#1} inv. = inventive/comp. = comparative
^{#2} cat 1 = Ti(IV)-tetraisobutylat/cat 2 = methanesulfonic acid/cat 3 = Zn-octanoate
^{#3} wt.-% relates to the educt mixture plus water.
^{#4} PEG = polyethylene glycol = polyethylene oxide with two primary OH end groups
^{#5} Calculated as described in the description of examples 11 to 22.
^{#6} The percent values relate to the CO₂ formation relative to the theoretical value.

Polymer backbone-Table 3; Esterification to PAG-Ester; Examples 23 to 29

| Example | Ex. 23 | Ex. 24 | Ex. 25 | Ex. 26 | Ex. 27 | Ex. 28 | Ex. 29 |
|---|---|---|---|---|---|---|---|
| Classification ^{#1} | inv. | inv. | inv. | inv. | inv. | inv. | comp. |
| Polymer backbone | B16 | B17 | B18 | B19 | B20 | B21 | |
| descriptor | | | | | | | |
| Educt mixture from | Ex. 16 | Ex. 17 | Ex. 18 | Ex. 19 | Ex. 20 | Ex. 21 | PEG |
| from ^{#2} | | | | | | | 9000 |
| Mₙ [g/mol] | 3700 | 4230 | 5150 | 4940 | 6400 | 5400 | 6830 |
| Biodegradation of the | 79 | 75 | 73 | 76 | 74 | 79 | 16 |
| Polymer backbone | | | | | | | |
| after 28 days | | | | | | | |
| according to | | | | | | | |
| OECD 301B [%] | | | | | | | |

^{#1} inv. = inventive/comp. = comparative
^{#2} PEG = polyethylene glycol = polyethylene oxide with two primary OH end groups

### Graft Polymer Examples

The following (general) procedures were performed using the starting material (“Educt”) and ratios and amounts as further indicated in Graft Polymer-Table 1.

Procedure for comparative example 1: graft polymerization of vinyl acetate on poly(ethylene glycol)—(Comp. Ex. 1; CE2 and 3 were prepared accordingly but with the respective amounts of monomers an ratios as shown in the Graf Polymer Table 1)

A polymerization vessel equipped with stirrer and reflux condenser was initially charged with 600 g of poly(ethylene glycol) under nitrogen atmosphere and melted at 90° C.

Feed 1 containing 4.8 g of tert-butyl peroxy-2-ethylhexanoate, dissolved in 23.6 g of tripropylene glycol, was dosed to the stirred vessel in 6:10 h, at 90° C. 5.56% of Feed 1 were dosed in the first 10 min and the rest was dosed with constant feed rate for 6:00 h. 10 minutes after the start of Feed 1, Feed 2 (400 g of vinyl acetate) was started and dosed within 6:00 h at constant feed rate and 90° C. Upon completion of the Feeds 1 and 2, the temperature was increased to 95° C. and Feed 3 consisting of 3.16 g of tert-butyl peroxy-2-ethylhexanoate, dissolved in 15.70 g of tripropylene glycol, were dosed within 56 min with constant flow rate at 95° C. The mixture was stirred for one hour at 95° C. upon complete addition of the feed.

Residual amounts of monomer were removed by vacuum distillation for 1 h at 95° C. and 500 mbar.

Graft polymers—general synthesis (details as shown in the Graft-Polymer-Table 1 below) In each of the examples G1-G20, 500 g of the polymer backbone Aare dosed in a 2,51 vessel equipped with an stainless steel anchor stirrer (and 2 other necks) and heated to 90° C. Dosage of the vinyl-monomers was started and continued over 6 h with constant feed rate. At the same time the Initiator t-butylperoxy-2-ethylhexanoate was dosed as a 26% solution in tripropylene glycol at a constant feed rate that in total lasted for 6 h and 45 minutes. For completion of reaction, the temperature was then increased to 105° C. and stirred for another 90 minutes. Finally, volatile components were stripped for 90 minutes at 115° C. with nitrogen at a feed rate of 6 L N2/h, followed by vacuum distillation at 60° C. and 40 mbar for approximately 1 hour for completion of volatile removal.

Further details such as monomer composition, ratios of polymer backbone A to monomer(s) B and results from the bio-degradation tests are shown in Graft Polymer—Table 1 containing basic relations employed for polymerization to obtain graft polymers/polymer compositions, and results from OECD 301F degradation tests.

| | Graft | | | | | | | | Phys. I | | | Bio- |
|---|---|---|---|---|---|---|---|---|---|---|---|---|
| | Polymer | Classifi- | Educt | Educt | Monomer | Monomer | Ratio | Initiator | properties | Mn | Mw | Degrad |
| Example | descriptor | cation | from | Descriptor | VM 1 | VM 2 | B/VM1/VM2 | [g] | 20° C. | g/mol | g/mol | [%] |
| Ex. 30 | G1 | inv | Ex. 11 | B1 | Vinyl | — | 50/50/0 | 67 | liquid | 2950 | 7200 | 69 |
| | | | | | acetate | | | | | | | |
| Ex. 31 | G2 | inv | Ex. 12 | B2 | Vinyl | Vinyl | 55/40/5 | 61 | liquid | 3980 | 9780 | 75 |
| | | | | | acetate | laurate | | | | | | |
| Ex. 32 | G3 | inv | Ex. 13 | B3 | Vinyl | — | 60/40 | 54 | liquid | 5100 | 18040 | 80.6 |
| | | | | | acetate | | | | | | | |
| Ex. 33 | G4 | inv | Ex. 13 | B3 | Vinyl | — | 40/60 | 58 | liquid | 6800 | 22900 | |
| | | | | | acetate | | | | | | | |
| Ex. 34 | G5 | inv | Ex. 15 | B4 | Vinyl | — | 55/45 | 55 | liquid | 7350 | 26850 | 69.5 |
| | | | | | propionate | | | | | | | |
| Ex. 35 | G6 | inv | Ex. 15 | B4 | Vinyl | Vinyl | 58/37/5 | 60 | | 7740 | 26480 | 74 |
| | | | | | acetate | laurate | | | | | | |
| Ex. 36 | G7 | inv | Ex. 16 | B5 | Vinyl | — | 40/60 | 57 | paste | 7950 | 29400 | 65 |
| | | | | | acetate | | | | | | | |
| Ex. 37 | G8 | inv | Ex. 16 | B5 | Vinyl | Vinyl | 60/30/10 | 63 | liquid | 6880 | 27460 | 73 |
| | | | | | acetate | octanoate | | | | | | |
| Ex. 38 | G9 | inv | Ex. 17 | B6 | Vinyl | — | 50/50 | 57 | paste | 7560 | 25780 | 69 |
| | | | | | acetate | | | | | | | |
| Ex. 39 | G10 | inv | Ex. 17 | B6 | Vinyl | Vinyl | 60/30/10 | 61 | paste | 7150 | 25120 | 74 |
| | | | | | acetate | laurate | | | | | | |
| Ex. 40 | G11 | inv | Ex. 17 | B6 | Vinyl | — | 65/35 | 50 | paste | 6440 | 21900 | 64 |
| | | | | | propionate | | | | | | | |
| Ex. 41 | G12 | inv | Ex. 20 | B9 | Vinyl | — | 45/55 | 55 | liquid | 13500 | 34650 | 62 |
| | | | | | acetate | | | | | | | |
| Ex. 42 | G13 | inv | Ex. 16 | B5 | Vinyl | — | 75/25 | 48 | paste | 4950 | 17160 | 67 |
| | | | | | acetate | | | | | | | |
| Ex. 43 | G14 | inv | Ex. 16 | B5 | Vinyl | Vinyl | 78/15/7 | 50 | paste | 4790 | 16900 | 65 |
| | | | | | acetate | octanoate | | | | | | |
| Ex. 44 | Comp C1 | comp | comp | PEG 4000 | Vinyl | — | 50/50 | 62 | solid | 6300 | 10010 | 23 |
| | | | | | acetate | | | | | | | |
| Ex. 45 | Comp C2 | comp | comp | PEG 6800 | Vinyl | — | 40/60 | 55 | solid | 9970 | 16930 | 14 |
| | | | | | acetate | | | | | | | |
| Ex. 46 | Comp C3 | comp | comp | PEG 6000 | Vinyl | — | 40/60 | 55 | solid | 10350 | 17280 | 28 |
| | | | | | acetate | | | | | | | |
| Ex. 47 | G15 | inv | Ex. 16 | B5 | V-acetate | Vinyl- | 60/30/10 | 61 | liquid | 3980 | 9780 | 75 |
| | | | | | | pyrolidone | | | | | | |
| Ex. 48 | G16 | inv | Ex. 15 | B4 | Vinyl | — | 55/45 | 55 | liquid | 7350 | 26850 | 69.5 |
| | | | | | propionate | | | | | | | |
| Ex. 49 | G17 | inv | Ex. 15 | B4 | Vinyl | V-laurate | 58/37/5 | 60 | | 7740 | 26480 | 74 |
| | | | | | acetate | | | | | | | |
| Ex. 50 | G18 | inv | Ex. 16 | B5 | Vinyl | — | 40/60 | 57 | paste | 7950 | 29400 | 65 |
| | | | | | acetete | | | | | | | |
| Ex. 51 | G19 | inv | Ex. 16 | B5 | Vinyl | V-octanoate | 60/30/10 | 63 | liquid | 6880 | 27460 | 73 |
| | | | | | acetate | | | | | | | |
| Ex. 52 | G20 | inv | Ex. 17 | B6 | Vinyl | — | 50/50 | 57 | paste | 7560 | 25780 | 69 |
| | | | | | acetate | | | | | | | |

“Educt”: the number is the number of the experiment on the production of the PAG-ester as described in the experimental section before.

Bio-degradation test results have been verified for the examples with the higher biodegradation percentages by at least one repetition. The results shown is the average number of those at least two tests.

### Performance Tests of Graft Polymer

Performance evaluations of the graft polymers can be obtained by laundry- and cleaning-experiments. Laundry experiments can be performed in washing machines or alternatively in equipment to perform model laundry experiments like Launderometer or Tergotometer. For testing of anti-redeposition effects, white fabrics were washed together with soiled fabrics in presence of a detergent composition containing the graft polymer and the remission of the white fabric is determined before and after the wash. For testing soil removal effects, soiled fabrics were washed in presence of a detergent composition containing the graft polymer and the remission of the soiled fabric is determined before and after the wash. Dosage of the graft polymer was chosen at 0.5 to 5% per weight of the detergent composition. Dosage of detergents was chosen in the range of 1500-4500 ppm in the wash liquor. Water hardness (Ca2+ and Mg2+ concentration in the wash liquor) in the wash experiments was set between 1 and 3 mmol hardness. Wash temperature was chosen between 20° C. and 40° C.

### Wash Performance

The wash performance (primary wash performance, secondary wash performance antigreying/antiredeposition) of the degradable, grafted polymers samples was tested in the launder-O-meter (11 beakers) by preparing wash solutions using water of 14° dH hardness (2.5 mmol/L; Ca:Mg:HCO3 4:1:8) containing 2.5 g/L of the liquid test detergent LD1 (see composition in table below) and always 2.0% of the inventive polymers.

Below in Detergent Tables 1 to 3 three liquid detergent formulations were chosen for differentiation of the graft polymers.

DETERGENT TABLE 1
Liquid detergent 1- LD1 (excellent detergent)

Liquid Detergent Formulation

| | |
|---|---|
| Sodium alkylbenzene sulfonic acid (C₁₀-C₁₃) LAS | 9.5% |
| C₁₃/C₁₅-Oxoalkohol reacted with 7 moles of EO | 4.5% |
| 1,2 propyleneglycol | 6% |
| ethanol | 2% |
| potassium coconut soap | 2.4% |
| NaOH | 2.2% |
| lauryl ether sulphate (Texapon) | 5.0% |
| Sodium citrate | 3% |
| Sokalan HP20 | 2% |
| Inventive polymer or comparison | 2% |
| Water | to 100% |

DETERGENT TABLE 2
Liquid detergent 2- LD2 (medium performance detergent)

| Liquid Detergent Formulation | |
|---|---|
| Sodium alkylbenzene sulfonic acid (C₁₀-C₁₃) | 5.5% |
| C₁₃/C₁₅-Oxoalkohol reacted with 7 moles of EO | 5.4% |
| 1,2 propyleneglycol | 6% |
| ethanol | 2% |
| potassium coconut soap | 2.4% |
| Monoethanolamine | 2.5% |
| lauryl ether sulphate | 5.4% |
| Sodium citrate | 3% |
| Sokalan HP96 | 2% |
| Inventive polymer or comparison | 2% |
| Water | to 100% |

DETERGENT TABLE 3
Liquid detergent 3- LD3 (medium
performance bio-based deterrent)

| Liquid Detergent Formulation | |
|---|---|
| MGDA | 5.5% |
| APG, branched C13 Glucosid | 3.5% |
| 1,2 propyleneglycol | 6% |
| ethanol | 2% |
| potassium coconut soap | 4.4% |
| NaOH | 2.2% |
| lauryl ether sulphate | 9.5% |
| Sodium citrate | 3% |
| Inventive polymer or comparison | 2% |
| Water | to 100% |

The first test criteria (primary wash performance) was selectively chosen to focus on sebum-cleaning properties, which is a primarily difficult stain (wfk 20D). This test was executed on two comparatively large 10 cm×10 cm textiles (wfk 20D)

Other criteria were those associated with the greying properties cotton and other textiles that is especially desired to avoid (anti-greying).

Anti greying tests were also executed in a launderometer with 11 beakers (LP2 type from SDL Atlas, Inc.). One wash cycle (60 min.) was run at 25° C. containing the wash-solution (0.25 L) together with one multi-stain monitor (MS1) and a cotton ballast fabric of 2.5 g (fabric to liquor ratio of 1:10). After the 1 cycle, the multi stain monitor was rinsed in water, followed by drying at ambient room temperature overnight. The multi-stain monitors MS1 and MS2 (shown below) contain respectively 8 and 4 standardized soiled fabrics, of respectively 5.0×5.0 cm and 4.5×4.5 cm size and stitched on two sides to a polyester carrier.

Multi-stain monitors used for evaluation of the cleaning performance (primary wash performance):

### MS1:

CFT C—S-10: butterfat with colorant on cotton
CFT C—S-62: lard, colored on cotton
CFT C—S-78: soybean oil with pigment on cotton
EMPA 112: cocoa on cotton
EMPA 141/1: lipstick on cotton
EMPA 125: soiling on cotton fabric, sensitive to surfactants as well as to lipases
wfk20D: pigment and sebum-type fat on polyester/cotton mixed fabric
CFT C—S-70: chocolate/mousse cream on cotton

### MS2:

CFT C—S-10: butterfat with colorant on cotton
CFT C—S-62: lard, colored on cotton
CFT C—S-61: beef fat, colored on cotton
CFT PC—S-04: Saturated with colored olive oil on Polyester/Cotton (65/35).

The total level of cleaning was evaluated using color measurements. Reflectance values of the stains on the monitors were measured using a sphere reflectance spectrometer (SF 500 type from Datacolor, USA, wavelength range 360-700 nm, optical geometry d/8°) with a UV cutoff filter at 460 nm. In this case, with the aid of the CIE-Lab color space classification, the brightness L *, the value a * on the red-green color axis and the b* value on the yellow-blue color axis, were measured before and after washing and averaged for the 8 stains of the monitor. The change of the color value (Delta E, ΔE) value, defined and calculated automatically by the evaluation color tools on the following formula ΔE=A Delta a * 2+Δ Delta b* 2+Δ Delta L*2, is a measure of the achieved cleaning effect. All experiments were repeated three times to yield an average number.

Higher Delta E values show better cleaning. For each stain, a difference of 1 unit can be detected visually by a skilled person. A non-expert can visually detect 2 units easily. The ΔE values of the formulations for the 8 stains of MS1 and for selected single stains are shown in Washing test-Table 1. Calculation of ΔE values is software-based, and it occurs automatically. In the launder-O-meter results, there is a trend towards a better cleaning performance.

Washing test-Table 1a: Results of launder-O-meter test fabric monitor - Formulation LD1

| | using | | ΔE | | | EMPA/SBL | | | Clay Slurry | | |
|---|---|---|---|---|---|---|---|---|---|---|---|
| | graft | | (CFT | | ΔE | | | Sum | | | Sum |
| | polymer | Total | C-S- | ΔE | (EMPA | Cotton | Polyester | BW + | Cotton | Polyester | BW + |
| Examples | of | ΔE | 62) | (wfk20D) | 141/1) | (BW) | (PES) | PES | (BW) | (PES) | PES |
| 53 | Ex. 44 | 143 | 34.0 | 12.6 | 13.3 | 25.3 | 22.8 | 48.1 | 24.4 | 20.3 | 44.7 |
| 54 | Ex. 45 | 147 | 33.5 | 13.6 | 15.1 | 27.3 | 23.0 | 50.3 | 25.1 | 22.7 | 47.8 |
| 55 | Ex. 30 | 145 | 33.0 | 13.0 | 14.5 | 24.9 | 24.4 | 49.3 | 24.3 | 23.6 | 47.9 |
| 56 | Ex. 31 | 149 | 34.0 | 13.5 | 13.9 | 23.1 | 26.4 | 49.5 | 19.5 | 27.1 | 46.6 |
| 57 | Ex. 32 | 147 | 35.0 | 13.2 | 15.6 | 25.0 | 23.5 | 48.5 | 20.7 | 25.6 | 46.3 |
| 58 | Ex. 33 | 154 | 37.2 | 15.5 | 17.0 | 27.0 | 25.2 | 52.2 | 24.9 | 26.3 | 51.2 |
| 59 | Ex. 34 | 146 | 32.7 | 12.0 | 13.5 | 22.4 | 24.6 | 47.0 | 24.0 | 25.8 | 49.8 |
| 60 | Ex. 35 | 149 | 34.5 | 14.6 | 15.9 | 22.7 | 28.4 | 51.1 | 24.1 | 26.0 | 50.1 |
| 61 | Ex. 36 | 151 | 35.5 | 16.6 | 14.7 | 26.0 | 27.1 | 53.1 | 23.9 | 26.9 | 50.8 |
| 62 | Ex. 37 | 149 | 33.5 | 13.5 | 14.5 | 22.0 | 25.7 | 47.7 | 25.6 | 22.4 | 48.0 |
| 63 | Ex. 38 | 149 | 36.0 | 15.5 | 13.9 | 25.5 | 26.2 | 51.7 | 24.4 | 25.7 | 50.1 |
| 64 | Ex. 39 | 150 | 36.0 | 14.0 | 16.6 | 23.9 | 25.3 | 49.2 | 23.7 | 24.9 | 48.6 |
| 65 | Ex. 40 | 147 | 34.0 | 15.5 | 16.0 | 22.1 | 23.7 | 45.8 | 23.2 | 25.9 | 49.1 |
| 66 | Ex. 41 | 146 | 33.8 | 13.0 | 14.5 | 26.5 | 25.3 | 51.4 | 25.7 | 24.9 | 50.6 |

Washing test-Table 1b - Formulation LD 2

| | using | | | | | EMPA/SBL | | | Clay Slurry | | |
|---|---|---|---|---|---|---|---|---|---|---|---|
| | graft | | ΔE | | ΔE | | Poly- | Sum | | Poly- | Sum |
| | polymer | Total | (CFT | ΔE | (EMPA | Cotton | ester | BW + | Cotton | ester | BW + |
| Example | of | ΔE | C-S-62) | (wfk20D) | 141/1) | (BW) | (PES) | PES | (BW) | (PES) | PES |
| 67 | Ex. 44 | 138 | 31.0 | 11.6 | 12.3 | 22.4 | 21.2 | 43.6 | 23.7 | 18.9 | 42.6 |
| 68 | Ex. 45 | 143 | 32.5 | 13.0 | 13.1 | 25.1 | 22.0 | 45.1 | 23.0 | 21.4 | 44.4 |
| 69 | Ex. 30 | 141 | 32.0 | 12.5 | 12.5 | 23.9 | 21.9 | 45.8 | 23.6 | 21.7 | 44.3 |
| 70 | Ex. 31 | 145 | 33.0 | 13.0 | 13.4 | 22.1 | 24.0 | 44.1 | 19.7 | 24.8 | 44.5 |
| 71 | Ex. 32 | 143 | 33.4 | 13.0 | 14.6 | 22.8 | 21.9 | 44.7 | 19.3 | 24.2 | 43.5 |
| 72 | Ex. 33 | 151 | 36.0 | 14.6 | 15.0 | 25.4 | 24.9 | 50.3 | 23.1 | 24.7 | 47.8 |
| 73 | Ex. 34 | 149 | 32.0 | 12.8 | 13.5 | 21.0 | 21.4 | 42.4 | 23.1 | 23.3 | 46.4 |
| 74 | Ex. 35 | 145 | 32.5 | 14.0 | 13.9 | 22.5 | 24.9 | 47.4 | 23.0 | 24.5 | 47.5 |
| 75 | Ex. 36 | 148 | 35.0 | 15.3 | 14.0 | 24.2 | 25.0 | 49.2 | 22.7 | 25.0 | 47.7 |
| 76 | Ex. 37 | 146 | 31.5 | 12.8 | 13.7 | 22.4 | 23.5 | 45.5 | 23.9 | 23.1 | 47.0 |
| 77 | Ex. 38 | 146 | 34.0 | 14.7 | 13.0 | 23.7 | 25.8 | 49.5 | 21.9 | 23.6 | 45.5 |
| 78 | Ex. 39 | 147 | 33.8 | 13.0 | 14.6 | 23.0 | 24.1 | 47.1 | 21.0 | 24.6 | 45.6 |
| 79 | Ex. 40 | 144 | 33.0 | 14.5 | 14.0 | 22.6 | 22.0 | 44.6 | 21.3 | 24.5 | 45.8 |
| 80 | Ex. 41 | 143 | 32.5 | 13.5 | 12.5 | 25.0 | 24.6 | 49.6 | 23.9 | 23.0 | 46.9 |

Washing test-Table 1c - Formulation LD3

| | using | | | | | EMPA/SBL | | | Clay Slurry | | |
|---|---|---|---|---|---|---|---|---|---|---|---|
| | graft | | ΔE | | ΔE | | Poly- | Sum | | Poly- | Sum |
| | polymer | Total | (CFT | ΔE | (EMPA | Cotton | ester | BW + | Cotton | ester | BW + |
| Example | of | ΔE | C-S-62) | (wfk20D) | 141/1) | (BW) | (PES) | PES | (BW) | (PES) | PES |
| 81 | Ex. 44 | 139 | 33.0 | 11.5 | 12.6 | 26.0 | 23.7 | 49.7 | 25.6 | 21.3 | 46.9 |
| 82 | Ex. 45 | 143 | 33.8 | 13.0 | 14.0 | 27.0 | 23.8 | 50.8 | 25.8 | 22.6 | 48.4 |
| 83 | Ex. 30 | 142 | 32.6 | 12.0 | 13.4 | 24.7 | 25.7 | 50.4 | 24.6 | 23.0 | 47.6 |
| 84 | Ex. 31 | 145 | 33.0 | 13.2 | 13.0 | 23.6 | 26.6 | 50.2 | 21.6 | 27.5 | 49.1 |
| 85 | Ex. 32 | 145 | 33.5 | 12.6 | 13.9 | 24.0 | 25.2 | 49.2 | 21.0 | 26.8 | 47.8 |
| 86 | Ex. 33 | 150 | 36.0 | 14.1 | 14.9 | 27.7 | 25.0 | 52.7 | 25.4 | 27.4 | 52.8 |
| 87 | Ex. 34 | 144 | 32.9 | 12.8 | 13.0 | 22.1 | 24.3 | 46.4 | 25.6 | 26.4 | 52.0 |
| 88 | Ex. 35 | 147 | 32.5 | 12.9 | 14.4 | 24.3 | 27.8 | 52.1 | 24.0 | 25.9 | 49.9 |
| 89 | Ex. 36 | 148 | 33.5 | 14.4 | 13.1 | 26.4 | 28.1 | 54.5 | 23.7 | 27.2 | 50.9 |
| 90 | Ex. 37 | 146 | 33.7 | 13.0 | 13.2 | 23.3 | 26.0 | 49.3 | 25.0 | 23.2 | 48.2 |
| 91 | Ex. 38 | 146 | 34.3 | 14.5 | 13.3 | 26.7 | 26.0 | 52.7 | 24.6 | 26.1 | 50.7 |
| 92 | Ex. 39 | 147 | 34.0 | 13.4 | 15.2 | 23.7 | 27.8 | 51.5 | 24.1 | 25.9 | 50.0 |
| 93 | Ex. 40 | 145 | 33.2 | 14.1 | 14.3 | 23.0 | 24.4 | 47.4 | 23.9 | 26.0 | 49.9 |
| 94 | Ex. 41 | 144 | 33.8 | 13.5 | 13.7 | 25.4 | 26.9 | 52.3 | 25.7 | 24.9 | 50.6 |
| 95 | Ex. 42 | 143 | 31.7 | 13.1 | 13.1 | 22.3 | 23.4 | 45.7 | 24.9 | 26.2 | 51.1 |
| 96 | Ex. 43 | 142 | 32.7 | 13.0 | 12.0 | 23.6 | 24.6 | 48.2 | 26.0 | 24.9 | 509 |

The excellent anti-greying properties of the inventive graft polymer were demonstrated by using the launder-o-meter in comparison to a compound of the prior art as follows:

Several white test swatches were washed together with soiled fabric EMPA 101/SBL 2004 and 20 steel balls at 40° C. in water with the selected composition comprising two or more compounds of formula (I) or comparative compound. The pH value of the washing liquor was adjusted to 8.0. The compositions comprising two or more compounds used as well as the comparative compounds are outlined in table 1. After the washing, the test fabrics were rinsed and spin-dried.

This washing cycle was repeated two times with new soiled fabric and new washing liquor. After the third wash, the test fabrics were rinsed, spin-dried and dried in the air.

The washing conditions are shown below (which was taken from a published patent application):

The washing condition are outlined in table 2 below.

TABLE 2
Washing conditions:

| Test equipment | Launder-o-meter, LP2 Typ, SDL Atlas Inc., USA |
|---|---|
| Washing liquor | 250 ml |
| Washing time/temperature | 20 min at 40° C. |
| Dosage | 1 g tested compound/L |
| Fabric/liquor ratio | 1:10 |
| Washing cycles | 3 |
| Water hardness | 2.5 mmol/l Ca²⁺:Mg²⁺:HCO₃₋ 4:1:8 |
| Soiling fabric | 2.5 g EMPA 101⁵⁾ |
| | 2.5 g SBL 2004⁶⁾ |
| | 2.5 g clay slurry⁷⁾ |
| Sum test + soiled fabric | 20 g |
| White test fabric, each 10 × 10 cm | wfk 10A, wfk 80A, wfk12A, EMPA 221¹⁾ |
| | wfk 20A²⁾ |
| | wfk 30A³⁾ |
| | EMPA 406 ⁴⁾ |

¹⁾Cotton fabrics: wfk 10A, Remission 81.8%; producer: wfk Testgewebe GmbH, Brüggen, Deutschland wfk 80A, Remission 85.7%; producer: wfk Testgewebe GmbH, Brüggen, Deutschland wfk 12A, Remission 94.4%; producer: wfk Testgewebe GmbH, Brüggen, Deutschland EMPA 221, Remission 87.1%; producer: EMPA Testmaterialien AG, Sankt Gallen, Schweiz
²⁾wfk 20 A Polyester/cotton, Remission 83.4%; producer: wfk Testgewebe GmbH, Brüggen, Deutschland
³⁾wfk 30 A Polyester, Remission 81.2%; producer: wfk Testgewebe GmbH, Brüggen, Deutschland
⁴⁾ EMPA 406 Polyamid, Remission 77.1%; producer: EMPA Testmaterialien AG, Sankt Gallen, Schweiz
⁵⁾EMPA 101, Carbon black/Olive oil; producer: producer: EMPA Testmaterialien AG, Sankt Gallen, Schweiz
⁶⁾SBL 2004, Soil load sheet; producer: wfk Testgewebe GmbH, Brüggen, Deutschland
⁷⁾mixture of clay, peanut oil, mineral oil and water

The antigreying performance was determined by measuring the remission value of the soiled fabric before and after wash with the spectrophotometer from Fa. Datacolor (Elrepho 2000) at 460 nm. The results are delta delta values, which means that the improved remission is compared with the results without the polymers. The higher the value, the better is the performance. The results are also outlined in Tables above. From the results, it can be gathered that the inventive compositions comprising two or more compounds of formula (I) show excellent anti-greying performance compared to compounds of the prior art.

### EXPERIMENTAL SECTION ON AGROCHEMICAL FORMULATIONS

### Agrochemical Example 1: Graft Polymer GX

### Step 1: Preparation of Polyethylene Glycol Monocarboxylic Acid

The procedure for examples 1 to 10 as outlined in the experimental section on the PAG-Ester polymers was essentially followed, using PEG 600 as starting material.

### Step 2: Preparation of Polyetherester BX

Polyethylene glycol monocarboxylic acid from the first step (100,0 g, 98% purity, 2% water, K-value: 10.4, acid value 47.68 mg KOH/g) was mixed with 0.4 g of Sn-octoate catalyst and heated for 96 hours with an increasing temperature*under vacuum 100 mbar at 125 to 145° C. acid number and K-value was monitored for the progress of the esterification reaction. was obtained with a K-value of 20.2 and a MW of approximately 3000-4000

### Step 3: Preparation of Graft Polymer GX

Graft polymer GX was prepared from the polyetherester BX obtained in the second step by reaction with vinyl acetate. To this end, 500 g of polyetherester (BX) are dosed in a 3 L vessel equipped with a stainless steel anchor stirrer (and 2 other necks) and heated to 95° C. Then, 800 g of vinyl acetate is dosed to the vessel over a time period of 7.5 hours with appropriate feed rates. Simultaneously with the start of dosing of vinyl acetate, the initiator t-butyl peroxy-2-ethylhexanoate is dosed to the vessel as a 27.8 wt % solution in tripropylene glycol in an amount of 59 g over a period of 8.5 hours). For completion of reaction, the temperature of the reaction mixture was then maintained at 95° C. for three hours. Finally, volatile components were stripped at 120° C. with nitrogen at a feed rate of 4 L N2/h. The resulting reaction mixture is then decanted at 60° C. The resulting graft polymer had a residual monomer content of 0.16 wt %.

### Example 2

Suspensions concentrates SC1 to SC7 according to Table X1 were prepared by grinding a composition comprising 40 wt % of an agrochemically active ingredient selected from azoxystrobin, atrazine, chlorothalonil, fluxapyroxad, diflufenican, terbutylazin and mefentrifluconoazol, 2.5% or 5 wt % of graft polymer GX, 0.3 wt %, and water (to 100 wt %) of a silicon-based anti-foaming agent in a disperser (DAS 200 by Lau GmbH, Germany) with glass balls (diameter 2 or 3 mm) such that the dispersed particles reach a particle size distribution characterized by a D90 value of up to 10 μm, a D50 value of up to 3 μm, and a D10 value of up to 1 μm. The measurement was performed as described under Example 3.

TABLE X1
active ingredients and graft polymer concentrations
of suspension concentrates SC1 to SC7

| Suspension | Active Ingredient; | Graft polymer | |
|---|---|---|---|
| Concentrate | concentration | GX concentration | |
| SC1 | Azoxystrobin (40 wt %) | 2.5 | wt % |
| SC2 | Azoxystrobin (40 wt %) | 5 | wt % |
| SC3 | Atrazine (40 wt %) | 2.5 | wt % |
| SC4 | Atrazine (40 wt %) | 5 | wt % |
| SC5 | Chlorothalonil (40 wt %) | 2.5 | wt % |
| SC6 | Chlorothalonil (40 wt %) | 5 | wt % |
| SC7 | Fluxapyroxad (40 wt %) | 5 | wt % |

### Example 3

The particle size distribution of Suspension Concentrates SC1 to SC7 was analyzed directly after preparation and after incubation for two weeks. The incubation was carried out according to CIPAC MT 46.3 either at 20 to 25° C., at 54° C., or at a cycling temperature of −10/+40° C. (temperature kept constant for 12 hours; total incubation time of 14 days). A volume of 10 ml of the respective suspension concentrate was placed in a 40 ml glass bottle fitted with screw cap and polyethylene inserts and kept in an oven at the specified temperature (+/−2° C.) for the defined period of time. Then the bottle was removed from the oven and allowed to reach 20 to 25° C. before further analysis.

The particle size measurement was carried out according to CIPAC MT 187 as follows. A volume of 1.0 ml of the respective Suspension Concentrate was stirred into 9 ml of fully demineralized water using a magnetic stirrer. Specific amounts of this diluted sample were added to the Malvern Master Sizer Dispersing Unit (Hydro MV) until a laser shadowing of 6% (+/−1,5%) was reached. Within the dispersing unit, the sample was diluted in 120 ml of fully demineralized water and pumped through the measuring cell of the Malvern Mastersizer 3000 (Malvern Pananalytical GmbH, Germany) that used a 632.8 nm laser (4 mW He—Ne) for analysis. The sample and the fully demineralized water used for the dilution were at 20 to 25° C. Particle size distribution, including D10, D50 and D90 values, was calculated using the Fraunhofer model as known in the art. See, e.g., ISO 13320-1:1999(E).

Tables X2 to X5 summarizes the results of these measurements.

TABLE X2
particle size measurements for Suspension
Concentrates SC1 and SC2

| | | |
|---|---|---|
| Time of measurement | SC1 | SC2 |

| | | | | |
|---|---|---|---|---|
| Start | D 10 | 0.56 μm | D 10 | 0.54 μm |
| | D 50 | 1.33 μm | D 50 | 1.25 μm |
| | D 90 | 4.15 μm | D 90 | 3.11 μm |
| 20-25° C., 14 days | D 10 | 0.53 μm | D 10 | 0.54 μm |
| | D 50 | 1.17 μm | D 50 | 1.23 μm |
| | D 90 | 2.54 μm | D 90 | 2.93 μm |

TABLE X3
particle size measurements for Suspension
Concentrates SC3 and SC4

| | | |
|---|---|---|
| Time of measurement | SC3 | SC4 |

| | | | | |
|---|---|---|---|---|
| Start | D 10 | 0.94 μm | D 10 | 0.83 μm |
| | D 50 | 2.20 μm | D 50 | 1.88 μm |
| | D 90 | 5.19 μm | D 90 | 4.67 μm |
| −10/+40° C., 14 days | D 10 | 0.76 μm | D 10 | 0.83 μm |
| | D 50 | 1.9 μm | D 50 | 1.9 μm |
| | D 90 | 4.92 μm | D 90 | 4.68 μm |

TABLE X4
particle size measurements for Suspension
Concentrates SC5 and SC6

| | |
|---|---|
| SC5 | SC6 |

| | | | | |
|---|---|---|---|---|
| start | D 10 | 0.64 μm | D 10 | 0.61 μm |
| | D 50 | 1.85 μm | D 50 | 1.77 μm |
| | D 90 | 5.88 μm | D 90 | 5.68 μm |
| −10/+40° C., 14 d | D 10 | 0.71 μm | D 10 | 0.52 μm |
| | D 50 | 2.13 μm | D 50 | 1.45 μm |
| | D 90 | 5.73 μm | D 90 | 3.56 μm |

TABLE X5
particle size measurements for Suspension Concentrate SC7

SC7

| | | |
|---|---|---|
| start | D 10 | 0.63 μm |
| | D 50 | 1.66 μm |
| | D 90 | 4.29 μm |
| rt, 14 d | D 10 | 0.65 μm |
| | D 50 | 1.73 μm |
| | D 90 | 4.36 μm |
| 54° C., 14 d | D 10 | 0.67 μm |
| | D 50 | 1.84 μm |
| | D 90 | 5.10 μm |

### Example 4

Suspensibility according to CIPAC MT 161 and blooming of Suspension Concentrates SC1 to SC7 were measured as follows. Samples of the Suspension Concentrates were analyzed either directly after preparation, or after incubation as described in Example 3. A total of 5g of the respective Suspension Concentrate was placed in a 100 ml measuring cylinder and filled with CIPAC water D to a total of 100g. When water is added the “blooming”, i.e. the degree of homogenous distribution, is evaluated. After that the cylinder is re-homogenized by ten times 180° inversion and allowed to stand for 30 min. Next, the top nine-tenths are removed and the remaining tenth is then assayed gravimetrically and the suspensibility is calculated. The results are summarized in Table X6.

Blooming is ranked according to the following grades: 1 is homogeneous; 3 means cylinder completely filled, but not completely homogeneous (<20%); 5 means Suspension Concentrate does not distribute, remains either at the top or at the bottom, 2 & 3 is accordingly in between.

The results are summarized in Tables X6 to X9.

TABLE X6
Suspensibility and blooming of SC1 and SC2

| | SC1 | SC2 |
|---|---|---|
| Start | | |
| Blooming | 2 | 2 |
| Suspensibility after 30 min | 95.7% | 95.6% |
| 20 to 25° C., 14 days | | |
| Blooming | 2 | 2 |
| Suspensibility after 30 min | 96.3% | 94.6% |

TABLE X7
Suspensibility and blooming of SC3 and SC4

| | SC3 | SC4 |
|---|---|---|
| Start | | |
| Blooming | 3 | 2-3 |
| Suspensibility after 30 min | 94.8% | 95.1% |
| −10/+40° C., 14 days | | |
| Blooming | 2-3 | 2-3 |
| Suspensibility after 30 min | 97.2% | 94.4% |

TABLE X8
Suspensibility and blooming of SC5 and SC6

| | SC5 | SC6 |
|---|---|---|
| Start | | |
| Blooming | 2 | 2 |
| Suspensibility after 30 min | 95.5% | 95.1% |
| −10/+40° C., 14 days | | |
| Blooming | 3 | 2-3 |
| Suspensibility after 30 min | 88.7% | 96.2% |

TABLE X9
Suspensibility and blooming of SC7

| | SC7 |
|---|---|
| Start | |
| Blooming | 2 |
| Suspensibility after 30 min | 94.4% |
| 20 to 25° C., 14 days | |
| Blooming | 2 |
| Suspensibility after 30 min | 96% |
| 54° C., 14 days | |
| Blooming | 2-3 |
| Suspensibility after 30 min | 95.5% |

Further results are shown in the following tables.

In the tables, in the upper left corner the polymer being employed as dispersant is listed, e.g. “Ex. 34” for the polymer from example 34.

In the upper right corner the amount the polymer is employed is listed, e.g. “5% w.s. dispersant” means that the polymer is employed as dispersant at a concentration of 5 weight percent by the total weight of the solids in the formulation in which it is tested.

In the tables the values measured for the particles sizes, blooming and suspension stability etc. were measured as described in the previous examples 2, 3 and 4 in this chapter on examples for the agrochemical applications.

Azoxystrobin

5% w.s. dispersant

| | | μm |
|---|---|---|
| EX. 32 | | |
| start | D 10 | 0.54 |
| | D 50 | 1.25 |
| | D 90 | 3.06 |
| rt, 14 d | D 10 | 0.53 |
| | D 50 | 1.23 |
| | D 90 | 2.93 |
| −10/+40° C., 14 d | D 10 | 0.60 |
| | D 50 | 1.46 |
| | D 90 | 3.09 |
| 54° C., 14 d | D 10 | 0.52 |
| | D 50 | 1.23 |
| | D 90 | 2.74 |
| EX. 30 | | |
| start | D 10 | 0.59 |
| | D 50 | 1.37 |
| | D 90 | 3.28 |
| rt, 14 d | D 10 | 0.57 |
| | D 50 | 1.33 |
| | D 90 | 3.09 |
| −10/+40° C., 14 d | D 10 | 0.63 |
| | D 50 | 1.69 |
| | D 90 | 3.64 |
| 54° C., 14 d | D 10 | 0.58 |
| | D 50 | 1.33 |
| | D 90 | 2.93 |
| EX. 33 | | |
| start | D 10 | 0.56 |
| | D 50 | 1.27 |
| | D 90 | 2.8 |
| rt, 14 d | D 10 | 0.56 |
| | D 50 | 1.26 |
| | D 90 | 2.74 |
| EX. 34 | | |
| start | D 10 | 0.82 |
| | D 50 | 2.18 |
| | D 90 | 4.70 |
| rt, 14 d | D 10 | 0.72 |
| | D 50 | 1.76 |
| | D 90 | 3.88 |
| EX. 41 | | |
| start | D 10 | 0.54 |
| | D 50 | 1.25 |
| | D 90 | 3.06 |
| rt, 14 d | D 10 | 0.52 |
| | D 50 | 1.22 |
| | D 90 | 2.97 |
| −10/+40° C., 14 d | D 10 | 0.60 |
| | D 50 | 1.43 |
| | D 90 | 2.98 |
| 54° C., 14 d | D 10 | 0.74 |
| | D 50 | 2.34 |
| | D 90 | 5.84 |
| EX. 38 | | |
| start | D 10 | 0.55 |
| | D 50 | 1.28 |
| | D 90 | 3.03 |
| rt, 14 d | D 10 | 0.51 |
| | D 50 | 1.18 |
| | D 90 | 2.72 |
| −10/+40° C., 14 d | D 10 | 0.62 |
| | D 50 | 1.69 |
| | D 90 | 3.73 |
| 54° C., 14 d | D 10 | 0.5 |
| | D 50 | 1.14 |
| | D 90 | 2.49 |
| EX. 47 | | |
| start | D 10 | 0.63 |
| | D 50 | 1.41 |
| | D 90 | 3.06 |
| rt, 14 d | D 10 | 0.68 |
| | D 50 | 1.5 |
| | D 90 | 3.2 |
| EX. 43 | | |
| start | D 10 | 0.62 |
| | D 50 | 1.38 |
| | D 90 | 3.07 |
| rt, 14 d | D 10 | 0.62 |
| | D 50 | 1.37 |
| | D 90 | 3.02 |
| 54° C., 14 d | D 10 | 0.47 |
| | D 50 | 0.94 |
| | D 90 | 2.48 |
| EX. 49 | | |
| start | D 10 | 0.63 |
| | D 50 | 1.46 |
| | D 90 | 3.36 |
| rt, 14 d | D 10 | 0.63 |
| | D 50 | 1.45 |
| | D 90 | 3.33 |
| −10/+40° C., 14 d | D 10 | 0.68 |
| | D 50 | 1.79 |
| | D 90 | 3.98 |
| 54° C., 14 d | D 10 | 0.63 |
| | D 50 | 1.48 |
| | D 90 | 3.26 |
| EX. 35 | | |
| start | D 10 | 0.62 |
| | D 50 | 1.43 |
| | D 90 | 3.29 |
| rt, 14 d | D 10 | 0.62 |
| | D 50 | 1.42 |
| | D 90 | 3.2 |
| −10/+40° C., 14 d | D 10 | 0.70 |
| | D 50 | 2.04 |
| | D 90 | 6.08 |
| EX. 31 | | |
| start | D 10 | 0.57 |
| | D 50 | 1.31 |
| | D 90 | 3.09 |
| rt, 14 d | D 10 | 0.57 |
| | D 50 | 1.28 |
| | D 90 | 2.9 |
| −10/+40° C., 14 d | D 10 | 0.68 |
| | D 50 | 2.06 |
| | D 90 | 9.18 |
| EX. 37 | | |
| start | D 10 | 0.6 |
| | D 50 | 1.31 |
| | D 90 | 2.96 |
| rt, 14 d | D 10 | 0.59 |
| | D 50 | 1.29 |
| | D 90 | 2.93 |
| −10/+40° C., 14 d | D 10 | 0.68 |
| | D 50 | 1.65 |
| | D 90 | 3.47 |
| 54° C., 14 d | D 10 | 0.60 |
| | D 50 | 1.3 |
| | D 90 | 2.77 |
| EX. 51 | | |
| start | D 10 | 0.61 |
| | D 50 | 1.32 |
| | D 90 | 2.99 |
| rt, 14 d | D 10 | 0.58 |
| | D 50 | 1.28 |
| | D 90 | 2.92 |
| −10/+40° C., 14 d | D 10 | 0.66 |
| | D 50 | 1.57 |
| | D 90 | 3.2 |
| 54° C., 14 d | D 10 | 0.59 |
| | D 50 | 1.3 |
| | D 90 | 2.8 |
| EX. 40 | | |
| start | D 10 | 0.59 |
| | D 50 | 1.31 |
| | D 90 | 3.14 |
| rt, 14 d | D 10 | 0.62 |
| | D 50 | 1.36 |
| | D 90 | 3.36 |
| −10/+40° C., 14 d | D 10 | 0.64 |
| | D 50 | 1.53 |
| | D 90 | 3.51 |
| 54° C., 14 d | D 10 | 0.56 |
| | D 50 | 1.05 |
| | D 90 | 2.05 |
| EX. 48 | | |
| start | D 10 | 0.56 |
| | D 50 | 1.25 |
| | D 90 | 2.95 |
| rt, 14 d | D 10 | 0.53 |
| | D 50 | 1.18 |
| | D 90 | 2.73 |
| −10/+40° C., 14 d | D 10 | 0.61 |
| | D 50 | 1.36 |
| | D 90 | 2.88 |
| 54° C., 14 d | D 10 | 0.53 |
| | D 50 | 1.15 |
| | D 90 | 2.49 |

Terbutylazin

5% w.s. dispersant

| | | μm |
|---|---|---|
| EX. 32 | | |
| start | D 10 | 0.76 |
| | D 50 | 1.76 |
| | D 90 | 4.46 |
| rt, 14 d | D 10 | 0.75 |
| | D 50 | 1.69 |
| | D 90 | 3.86 |
| 54° C., 14 d | D 10 | 0.8 |
| | D 50 | 1.81 |
| | D 90 | 4.18 |
| EX. 30 | | |
| start | D 10 | 0.78 |
| | D 50 | 1.82 |
| | D 90 | 4.89 |
| rt, 14 d | D 10 | 0.78 |
| | D 50 | 1.79 |
| | D 90 | 4.5 |
| 54° C., 14 d | D 10 | 0.82 |
| | D 50 | 1.88 |
| | D 90 | 4.53 |
| EX. 33 | | |
| start | D 10 | 0.77 |
| | D 50 | 1.77 |
| | D 90 | 4.53 |
| rt, 14 d | D 10 | 0.78 |
| | D 50 | 1.75 |
| | D 90 | 4.16 |
| 54° C., 14 d | D 10 | 0.80 |
| | D 50 | 1.85 |
| | D 90 | 4.39 |
| EX. 34 | | |
| start | D 10 | 0.96 |
| | D 50 | 2.26 |
| | D 90 | 4.83 |
| EX. 41 | | |
| start | D 10 | 0.74 |
| | D 50 | 1.69 |
| | D 90 | 3.99 |
| rt, 14 d | D 10 | 0.76 |
| | D 50 | 1.68 |
| | D 90 | 3.84 |
| 54° C., 14 d | D 10 | 0.79 |
| | D 50 | 1.75 |
| | D 90 | 3.86 |
| EX. 38 | | |
| start | D 10 | 0.741 |
| | D 50 | 1.68 |
| | D 90 | 3.94 |
| rt, 14 d | D 10 | 0.76 |
| | D 50 | 1.7 |
| | D 90 | 4.11 |
| 54° C., 14 d | D 10 | 0.78 |
| | D 50 | 1.76 |
| | D 90 | 3.98 |
| EX. 47 | | |
| start | D 10 | 0.92 |
| | D 50 | 2.32 |
| | D 90 | 6.35 |
| EX. 43 | | |
| start | D 10 | 0.95 |
| | D 50 | 2.39 |
| | D 90 | 5.79 |
| EX. 49 | | |
| start | D 10 | 0.78 |
| | D 50 | 1.77 |
| | D 90 | 3.91 |
| rt, 14 d | D 10 | 0.79 |
| | D 50 | 1.78 |
| | D 90 | 3.88 |
| −10/+40° C., 14 d | D 10 | 0.92 |
| | D 50 | 2.43 |
| | D 90 | 6.2 |
| 54° C., 14 d | D 10 | 0.84 |
| | D 50 | 1.89 |
| | D 90 | 4.14 |
| EX. 35 | | |
| start | D 10 | 0.84 |
| | D 50 | 2.02 |
| | D 90 | 4.77 |
| rt, 14 d | D 10 | 0.83 |
| | D 50 | 1.97 |
| | D 90 | 4.49 |
| −10/+40° C., 14 d | D 10 | 0.91 |
| | D 50 | 2.51 |
| | D 90 | 5.87 |
| 54° C., 14 d | D 10 | 0.89 |
| | D 50 | 2.12 |
| | D 90 | 4.8 |
| EX. 31 | | |
| start | D 10 | 0.77 |
| | D 50 | 1.73 |
| | D 90 | 3.85 |
| rt, 14 d | D 10 | 0.79 |
| | D 50 | 1.74 |
| | D 90 | 3.78 |
| −10/+40° C., 14 d | D 10 | 0.90 |
| | D 50 | 2.41 |
| | D 90 | 6.96 |
| 54° C., 14 d | D 10 | 0.84 |
| | D 50 | 1.85 |
| | D 90 | 3.95 |

Diflufenican

5% w.s. dispersant

| | | μm |
|---|---|---|
| EX. 30 | | |
| start | D 10 | 0.781 |
| | D 50 | 2.00 |
| | D 90 | 5.33 |
| rt, 14 d | D 10 | 0.75 |
| | D 50 | 1.89 |
| | D 90 | 4.50 |
| −10/+40° C., 14 d | D 10 | 0.8 |
| | D 50 | 2.37 |
| | D 90 | 5.81 |
| 54° C., 14 d | D 10 | 0.77 |
| | D 50 | 2.15 |
| | D 90 | 4.84 |
| EX. 33 | | |
| start | D 10 | 0.80 |
| | D 50 | 2.08 |
| | D 90 | 5.44 |
| rt, 14 d | D 10 | 0.77 |
| | D 50 | 1.98 |
| | D 90 | 4.75 |
| 54° C., 14 d | D 10 | 0.78 |
| | D 50 | 2.24 |
| | D 90 | 5.07 |
| EX. 41 | | |
| start | D 10 | 0.77 |
| | D 50 | 1.98 |
| | D 90 | 5.14 |
| rt, 14 d | D 10 | 0.74 |
| | D 50 | 1.86 |
| | D 90 | 4.39 |
| −10/+40° C., 14 d | D 10 | 0.79 |
| | D 50 | 2.28 |
| | D 90 | 5.52 |
| 54° C., 14 d | D 10 | 0.76 |
| | D 50 | 2.11 |
| | D 90 | 4.78 |
| EX. 38 | | |
| start | D 10 | 0.78 |
| | D 50 | 2.00 |
| | D 90 | 5.11 |
| rt, 14 d | D 10 | 0.74 |
| | D 50 | 1.89 |
| | D 90 | 4.46 |
| 54° C., 14 d | D 10 | 0.78 |
| | D 50 | 2.18 |
| | D 90 | 4.93 |
| EX. 47 | | |
| start | D 10 | 0.81 |
| | D 50 | 2.05 |
| | D 90 | 4.68 |
| EX. 43 | | |
| start | D 10 | 1.00 |
| | D 50 | 2.05 |
| | D 90 | 4.71 |
| EX. 49 | | |
| start | D 10 | 0.78 |
| | D 50 | 2.04 |
| | D 90 | 4.94 |
| rt, 14 d | D 10 | 0.77 |
| | D 50 | 2.04 |
| | D 90 | 4.74 |
| −10/+40° C., 14 d | D 10 | 0.85 |
| | D 50 | 2.64 |
| | D 90 | 6.58 |
| 54° C., 14 d | D 10 | 0.82 |
| | D 50 | 2.38 |
| | D 90 | 5.37 |
| EX. 35 | | |
| start | D 10 | 0.83 |
| | D 50 | 2.18 |
| | D 90 | 5.53 |
| rt, 14 d | D 10 | 0.80 |
| | D 50 | 2.10 |
| | D 90 | 4.99 |
| −10/+40° C., 14 d | D 10 | 0.86 |
| | D 50 | 2.65 |
| | D 90 | 7.10 |
| 54° C., 14 d | D 10 | 0.84 |
| | D 50 | 2.42 |
| | D 90 | 5.56 |
| EX. 31 | | |
| start | D 10 | 0.76 |
| | D 50 | 1.95 |
| | D 90 | 4.75 |
| rt, 14 d | D 10 | 0.75 |
| | D 50 | 1.95 |
| | D 90 | 4.59 |
| −10/+40° C., 14 d | D 10 | 0.83 |
| | D 50 | 2.52 |
| | D 90 | 6.23 |
| 54° C., 14 d | D 10 | 0.78 |
| | D 50 | 2.21 |
| | D 90 | 5.01 |

Mefentrifluconazol

5% w.s. dispersant

| | | μm |
|---|---|---|
| EX. 32 | | |
| start | D 10 | 0.63 |
| | D 50 | 1.44 |
| | D 90 | 3.45 |
| rt, 14 d | D 10 | 0.63 |
| | D 50 | 1.42 |
| | D 90 | 3.30 |
| −10/+40° C., 14 d | D 10 | 0.72 |
| | D 50 | 1.70 |
| | D 90 | 3.56 |
| 54° C., 14 d | D 10 | 0.70 |
| | D 50 | 1.52 |
| | D 90 | 3.28 |
| EX. 30 | | |
| start | D 10 | 0.65 |
| | D 50 | 1.48 |
| | D 90 | 3.86 |
| rt, 14 d | D 10 | 0.64 |
| | D 50 | 1.42 |
| | D 90 | 3.27 |
| −10/+40° C., 14 d | D 10 | 0.76 |
| | D 50 | 2.08 |
| | D 90 | 4.87 |
| 54° C., 14 d | D 10 | 0.70 |
| | D 50 | 1.52 |
| | D 90 | 3.30 |
| EX. 41 | | |
| start | D 10 | 0.612 |
| | D 50 | 1.39 |
| | D 90 | 3.31 |
| rt, 14 d | D 10 | 0.62 |
| | D 50 | 1.39 |
| | D 90 | 3.22 |
| −10/+40° C., 14 d | D 10 | 0.74 |
| | D 50 | 1.82 |
| | D 90 | 3.86 |
| 54° C., 14 d | D 10 | 0.69 |
| | D 50 | 1.49 |
| | D 90 | 3.20 |
| EX. 49 | | |
| start | D 10 | 0.86 |
| | D 50 | 2.50 |
| | D 90 | 4.66 |
| rt, 14 d | D 10 | 0.82 |
| | D 50 | 2.34 |
| | D 90 | 4.51 |
| −10/+40° C., 14 d | D 10 | 0.89 |
| | D 50 | 2.68 |
| | D 90 | 5.15 |
| 54° C., 14 d | D 10 | 0.90 |
| | D 50 | 2.76 |
| | D 90 | 5.45 |

Suspensibility and Blooming:
a) 40% w.s. Azoxystrobin

| | 5% w.s. |
|---|---|
| | dispersant |
| Ex. 32 | |
| start | |
| Blooming | 1.5 |
| Suspensibility | 100.3 |
| after 30 min | |
| rt, 14 d | |
| Blooming | 1.5 |
| Suspensibility | 99.8 |
| after 30 min | |
| −10/+40° C., 14 d | |
| Blooming | 2.5 |
| Suspensibility | 97.5 |
| after 30 min | |
| 54° C., 14 d | |
| Blooming | 2 |
| Suspensibility | 101.4 |
| after 30 min | |
| Ex. 30 | |
| start | |
| Blooming | 1.5 |
| Suspensibility | 100.7 |
| after 30 min | |
| rt, 14 d | |
| Blooming | 1.5 |
| Suspensibility | 99.6 |
| after 30 min | |
| −10/+40° C., 14 d | |
| Blooming | 2 |
| Suspensibility | 99.1 |
| after 30 min | |
| 54° C., 14 d | |
| Blooming | 2 |
| Suspensibility | 100.2 |
| after 30 min | |
| EX. 33 | |
| start | |
| Blooming | 3.5 |
| Suspensibility | 100.6 |
| after 30 min | |
| rt, 14 d | |
| Blooming | 1.5 |
| Suspensibility | 97.5 |
| after 30 min | |
| −10/+40° C., 14 d | |
| Blooming | 2.5 |
| Suspensibility | 82.3 |
| after 30 min | |
| EX. 34 | |
| start | |
| Blooming | 2 |
| Suspensibility | 100.7 |
| after 30 min | |
| rt, 14 d | |
| Blooming | 1.5 |
| Suspensibility | 96.9 |
| after 30 min | |
| EX. 41 | |
| start | |
| Blooming | 1.5 |
| Suspensibility | 101.0 |
| after 30 min | |
| rt, 14 d | |
| Blooming | 1.5 |
| Suspensibility | 100.2 |
| after 30 min | |
| −10/+40° C., 14 d | |
| Blooming | 2 |
| Suspensibility | 99.8 |
| after 30 min | |
| 54° C., 14 d | |
| Blooming | 2 |
| Suspensibility | 99.1 |
| after 30 min | |
| Ex. 38 | |
| start | |
| Blooming | 1.5 |
| Suspensibility | 101.0 |
| after 30 min | |
| rt, 14 d | |
| Blooming | 1.5 |
| Suspensibility | 100.0 |
| after 30 min | |
| −10/+40° C., 14 d | |
| Blooming | 2 |
| Suspensibility | 97.8 |
| after 30 min | |
| 54° C., 14 d | |
| Blooming | 2 |
| Suspensibility | 101.0 |
| after 30 min | |
| EX. 47 | |
| start | |
| Blooming | 3.5 |
| Suspensibility | 101.5 |
| after 30 min | |
| rt, 14 d | |
| Blooming | 2.5 |
| Suspensibility | 99.5 |
| after 30 min | |
| EX. 43 | |
| start | |
| Blooming | 2.5 |
| Suspensibility | 97.5 |
| after 30 min | |
| rt, 14 d | |
| Blooming | 1.5 |
| Suspensibility | 97.6 |
| after 30 min | |
| 54° C., 14 d | |
| Blooming | 2 |
| Suspensibility | 99.5 |
| after 30 min | |
| Ex. 49 | |
| start | |
| Blooming | 2 |
| Suspensibility | 100.4 |
| after 30 min | |
| rt, 14 d | |
| Blooming | 1.5 |
| Suspensibility | 99.4 |
| after 30 min | |
| −10/+40° C., 14 d | |
| Blooming | 2.5 |
| Suspensibility | 91.5 |
| after 30 min | |
| 54° C., 14 d | |
| Blooming | 2 |
| Suspensibility | 99.2 |
| after 30 min | |
| Ex. 35 | |
| start | |
| Blooming | 3.5 |
| Suspensibility | 99.9 |
| after 30 min | |
| rt, 14 d | |
| Blooming | 2.5 |
| Suspensibility | 99.9 |
| after 30 min | |
| −10/+40° C., 14 d | |
| Blooming | 3.5 |
| Suspensibility | 81.0 |
| after 30 min | |
| Ex. 31 | |
| start | |
| Blooming | 2 |
| Suspensibility | 100.3 |
| after 30 min | |
| rt, 14 d | |
| Blooming | 1.5 |
| Suspensibility | 99.4 |
| after 30 min | |
| −10/+40° C., 14 d | |
| Blooming | 2.5 |
| Suspensibility | 81.2 |
| after 30 min | |
| Ex. 37 | |
| start | |
| Blooming | 2 |
| Suspensibility | 100.2 |
| after 30 min | |
| rt, 14 d | |
| Blooming | 2 |
| Suspensibility | 99.7 |
| after 30 min | |
| −10/+40° C., 14 d | |
| Blooming | 2.5 |
| Suspensibility | 94.2 |
| after 30 min | |
| 54° C., 14 d | |
| Blooming | 2.5 |
| Suspensibility | 100.4 |
| after 30 min | |
| Ex. 51 | |
| start | |
| Blooming | 2 |
| Suspensibility | 100.0 |
| after 30 min | |
| rt, 14 d | |
| Blooming | 1.5 |
| Suspensibility | 100.1 |
| after 30 min | |
| −10/+40° C., 14 d | |
| Blooming | 2.5 |
| Suspensibility | 99.7 |
| after 30 min | |
| 54° C., 14 d | |
| Blooming | 2.5 |
| Suspensibility | 100.7 |
| after 30 min | |
| Ex. 40 | |
| start | |
| Blooming | 2 |
| Suspensibility | 100.2 |
| after 30 min | |
| rt, 14 d | |
| Blooming | 2 |
| Suspensibility | 99.7 |
| after 30 min | |
| −10/+40° C., 14 d | |
| Blooming | 2.5 |
| Suspensibility | 99.7 |
| after 30 min | |
| 54° C., 14 d | |
| Blooming | 2.5 |
| Suspensibility | 100.9 |
| after 30 min | |
| Ex. 48 | |
| start | |
| Blooming | 1.5 |
| Suspensibility | 100.4 |
| after 30 min | |
| rt, 14 d | |
| Blooming | 2 |
| Suspensibility | 100.3 |
| after 30 min | |
| −10/+40° C., 14 d | |
| Blooming | 2.5 |
| Suspensibility | 100.3 |
| after 30 min | |
| 54° C., 14 d | |
| Blooming | 2.5 |
| Suspensibility | 100.0 |
| after 30 min | |

b) 40% w.s. Terbutylazin

| | 5% w.s. |
|---|---|
| | dispersant |
| Ex. 32 | |
| start | |
| Blooming | 3 |
| Suspensibility | 99.9 |
| after 30 min | |
| rt, 14 d | |
| Blooming | 3 |
| Suspensibility | 99.6 |
| after 30 min | |
| 54° C., 14 d | |
| Blooming | 2 |
| Suspensibility | 98.2 |
| after 30 min | |
| Ex. 30 | |
| start | |
| Blooming | 1.5 |
| Suspensibility | 100.4 |
| after 30 min | |
| rt, 14 d | |
| Blooming | 1.5 |
| Suspensibility | 100.4 |
| after 30 min | |
| 54° C., 14 d | |
| Blooming | 1 |
| Suspensibility | 99.2 |
| after 30 min | |
| EX. 33 | |
| start | |
| Blooming | 3 |
| Suspensibility | 100.1 |
| after 30 min | |
| rt, 14 d | |
| Blooming | 2.5 |
| Suspensibility | 99.3 |
| after 30 min | |
| 54° C., 14 d | |
| Blooming | 1.5 |
| Suspensibility | 99.1 |
| after 30 min | |
| EX. 41 | |
| start | |
| Blooming | 2.5 |
| Suspensibility | 99.5 |
| after 30 min | |
| rt, 14 d | |
| Blooming | 3 |
| Suspensibility | 99.3 |
| after 30 min | |
| 54° C., 14 d | |
| Blooming | 2 |
| Suspensibility | 100.5 |
| after 30 min | |
| Ex. 38 | |
| start | |
| Blooming | 1.5 |
| Suspensibility | 100.3 |
| after 30 min | |
| rt, 14 d | |
| Blooming | 3.5 |
| Suspensibility | 100.5 |
| after 30 min | |
| 54° C., 14 d | |
| Blooming | 2 |
| Suspensibility | 100.1 |
| after 30 min | |
| EX. 47 | |
| start | |
| Blooming | 4.5 |
| Suspensibility | 100.8 |
| after 30 min | |
| EX. 43 | |
| start | |
| Blooming | 5 |
| Suspensibility | 99.3 |
| after 30 min | |
| Ex. 49 | |
| start | |
| Blooming | 3.5 |
| Suspensibility | 99.9 |
| after 30 min | |
| rt, 14 d | |
| Blooming | 4 |
| Suspensibility | 99.7 |
| after 30 min | |
| −10/+40° C., 14 d | |
| Blooming | 4 |
| Suspensibility | 93.1 |
| after 30 min | |
| 54° C., 14 d | |
| Blooming | 3 |
| Suspensibility | 99.0 |
| after 30 min | |
| EX. 35 | |
| start | |
| Blooming | 4.5 |
| Suspensibility | 100.3 |
| after 30 min | |
| rt, 14 d | |
| Blooming | 4 |
| Suspensibility | 99.4 |
| after 30 min | |
| −10/+40° C., 14 d | |
| Blooming | 4 |
| Suspensibility | 99.7 |
| after 30 min | |
| 54° C., 14 d | |
| Blooming | 2 |
| Suspensibility | 98.2 |
| after 30 min | |
| EX. 31 | |
| start | |
| Blooming | 3 |
| Suspensibility | 99.9 |
| after 30 min | |
| rt, 14 d | |
| Blooming | 3 |
| Suspensibility | 99.9 |
| after 30 min | |
| −10/+40° C., 14 d | |
| Blooming | 3.5 |
| Suspensibility | 90.4 |
| after 30 min | |
| 54° C., 14 d | |
| Blooming | 2 |
| Suspensibility | 99.1 |
| after 30 min | |

c) 40% ws Diflufenican

| | 5% w.s. |
|---|---|
| | dispersant |
| Ex. 30 | |
| start | |
| Blooming | 1 |
| Suspensibility | 98.8 |
| after 30 min | |
| rt, 14 d | |
| Blooming | 1 |
| Suspensibility | 98.2 |
| after 30 min | |
| −10/+40° C., 14 d | |
| Blooming | 1 |
| Suspensibility | 93.8 |
| after 30 min | |
| 54° C., 14 d | |
| Blooming | 1 |
| Suspensibility | 97.9 |
| after 30 min | |
| EX. 33 | |
| start | |
| Blooming | 1 |
| Suspensibility | 99.5 |
| after 30 min | |
| EX. 41 | |
| start | |
| Blooming | 1 |
| Suspensibility | 98.0 |
| after 30 min | |
| rt, 14 d | |
| Blooming | 1.5 |
| Suspensibility | 97.8 |
| after 30 min | |
| −10/+40° C., 14 d | |
| Blooming | 1 |
| Suspensibility | 95.7 |
| after 30 min | |
| 54° C., 14 d | |
| Blooming | 1 |
| Suspensibility | 97.8 |
| after 30 min | |
| EX. 38 | |
| start | |
| Blooming | 1 |
| Suspensibility | 98.4 |
| after 30 min | |
| EX. 47 | |
| start | |
| Blooming | 1.5 |
| Suspensibility | 98.0 |
| after 30 min | |
| EX. 43 | |
| start | |
| Blooming | 1 |
| Suspensibility | 98.4 |
| after 30 min | |
| Ex. 49 | |
| start | |
| Blooming | 1 |
| Suspensibility | 98.4 |
| after 30 min | |
| rt, 14 d | |
| Blooming | 1 |
| Suspensibility | 97.3 |
| after 30 min | |
| −10/+40° C., 14 d | |
| Blooming | 1.5 |
| Suspensibility | 94.9 |
| after 30 min | |
| 54° C., 14 d | |
| Blooming | 1.5 |
| Suspensibility | 96.5 |
| after 30 min | |
| Ex. 35 | |
| start | |
| Blooming | 3.5 |
| Suspensibility | 98.9 |
| after 30 min | |
| rt, 14 d | |
| Blooming | 3 |
| Suspensibility | 97.8 |
| after 30 min | |
| −10/+40° C., 14 d | |
| Blooming | 3.5 |
| Suspensibility | 95.2 |
| after 30 min | |
| 54° C., 14 d | |
| Blooming | 2 |
| Suspensibility | 96.4 |
| after 30 min | |
| Ex. 31 | |
| start | |
| Blooming | 1.5 |
| Suspensibility | 98.8 |
| after 30 min | |
| rt, 14 d | |
| Blooming | 1 |
| Suspensibility | 96.5 |
| after 30 min | |
| −10/+40° C., 14 d | |
| Blooming | 1.5 |
| Suspensibility | 89.6 |
| after 30 min | |
| 54° C., 14 d | |
| Blooming | 1 |
| Suspensibility | 96.5 |
| after 30 min | |

d) 40% ws Mefentrifluconazol

| | 5% w.s. |
|---|---|
| | dispersant |
| Ex. 32 | |
| start | |
| Blooming | 1 |
| Suspensibility | 100.1 |
| after 30 min | |
| rt, 14 d | |
| Blooming | 1.5 |
| Suspensibility | 100.0 |
| after 30 min | |
| −10/+40° C., 14 d | |
| Blooming | 1.5 |
| Suspensibility | 100.2 |
| after 30 min | |
| 54° C., 14 d | |
| Blooming | 1.5 |
| Suspensibility | 99.3 |
| after 30 min | |
| Ex. 30 | |
| start | |
| Blooming | 1 |
| Suspensibility | 99.9 |
| after 30 min | |
| rt, 14 d | |
| Blooming | 1 |
| Suspensibility | 99.5 |
| after 30 min | |
| −10/+40° C., 14 d | |
| Blooming | 1 |
| Suspensibility | 97.0 |
| after 30 min | |
| 54° C., 14 d | |
| Blooming | 1 |
| Suspensibility | 99.6 |
| after 30 min | |
| EX. 41 | |
| start | |
| Blooming | 1 |
| Suspensibility | 100.3 |
| after 30 min | |
| rt, 14 d | |
| Blooming | 1.5 |
| Suspensibility | 100.0 |
| after 30 min | |
| −10/+40° C., 14 d | |
| Blooming | 1.5 |
| Suspensibility | 98.7 |
| after 30 min | |
| 54° C., 14 d | |
| Blooming | 1.5 |
| Suspensibility | 99.0 |
| after 30 min | |
| EX. 49 | |
| start | |
| Blooming | 3 |
| Suspensibility | 99.9 |
| after 30 min | |
| rt, 14 d | |
| Blooming | 2 |
| Suspensibility | 96.8 |
| after 30 min | |
| −10/+40° C., 14 d | |
| Blooming | 2 |
| Suspensibility | 98.4 |
| after 30 min | |
| 54° C., 14 d | |
| Blooming | 1.5 |
| Suspensibility | 99.6 |
| after 30 min | |

1. A graft polymer comprising
(A) a polymer backbone as a graft base, wherein said polymer backbone (A) is a polyalkylene oxide ester polymer with a weight average molecular weight M_{w} of 500 to 50 000 g/mol and a polydispersity PD of 2 to 6, comprising 10 to 560 ether groups and 2 to 51 ester groups, which are interconnected with alkylene groups, wherein the polyalkylene oxide ester polymer contains 1 to 51 structural elements of the general formula (I) 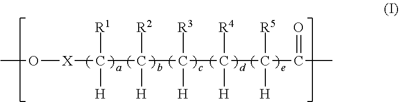
wherein
the —O—unit at the left side is bound to a —CO—unit of an adjacent unit of the polymer, forming an ester unit,
the —CO—unit at the right side is bound to a —O—unit of an adjacent unit of the polymer, forming a further ester unit,
R¹, R², R³, R⁴, and R⁵ represent independent of each other a hydrogen atom or a C₁₋₁₂ alkyl group,
a, b, c, d, and e represent independent of each other an integer of 0 or 1, whereas the sum of a to e is 1 to 5, and
X represents a polyalkylene oxide unit with 4 to 100 alkylene oxide units, wherein the alkylene oxide units contain independent of each other 2 to 6 carbon atoms in the direct chain between two —O—units, wherein each of the carbon atoms in the direct chain between two —O—units contain independent of each other either two hydrogen atoms, or one hydrogen atom and one C₁₋₁₂ alkyl group,
and
(B) polymeric sidechains grafted onto the polymer backbone A, wherein said polymeric sidechains (B) are obtainable by polymerization of at least one monomer selected from the group consisting of i) at least one vinyl ester monomer (B1) and ii) optionally at least one further olefinically unsaturated monomer (B2) polymerizable with monomer B1.

2. (canceled)

3. (canceled)

4. (canceled)

5. (canceled)

6. (canceled)

7. (canceled)

8. (canceled)

9. (canceled)

10. The graft polymer according to claim 1, comprising
(A) a polymer backbone as a graft base, wherein said polymer backbone (A) is obtainable by condensation comprising
i) diols of poly alkylene oxides (PAG-DO),
ii) di-carboxylic acids of PAG (PAG-DC), and/or
iii) mono-carbonic acid mono-ols of PAG (PAG-MC),
wherein either at least a compound selected from iii) is present, or—when only i) and ii) are present—at least two internal ester-groups are present, and
with the PAG being obtained by polymerization of at least one monomer selected from h EroUD consisting of 1,2-alkylene oxides, 1,4-diols, or their cyclic or oligomeric analogs; or the PAG based on the group consisting of polymeric ethers of such 1,4-diols, 1,6-diols, or their cyclic or oligomeric analogs; or the PAG based on polymeric ethers of such 1,6-diols; or any of their mixtures in any ratio, either as blocks of certain polymeric units, or as statistical polymeric structures, or as polymers comprising one or more homo-blocks of a certain monomer and one or more statistical blocks comprising more than one monomer, and any combination thereof such as polymers having several different blocks of different monomers, or blocks of two different monomers, blocks of statistical mixtures of two or more monomers,
and optionally other non-polymeric di-carboxylic acid compounds which may be present in addition to compound ii), and
(B) polymeric sidechains grafted onto the polymer backbone, wherein said polymeric sidechains (B) are obtainable by radical polymerization of at least one monomer being selected from the group consisting of i) at least one vinyl ester monomer (B1) and ii) optionally at least one further olefinically unsaturated monomer (B2) polymerizable with monomer B1.

11. The graft polymer according to claim 10, wherein the polymer backbone A has a weight average molecular weight Mw of 500 to 50 000 g/mol and a polydispersity PD of 2 to 6, comprising 10 to 560 ether groups and 2 to 51 ester groups, which are interconnected with alkylene groups, and wherein further low molecular weight di-carbonic acids may be contained in addition to the compounds iii).

12. The graft polymer according to claim 1, comprising
(A) polymer backbone A obtainable from at least one different polymer subunit linked by covalent ester-bonds, and optionally containing further low-molecular weight di-carboxylic acid compounds, wherein the polymer backbone is obtainable from the esterification reaction of
a) at least one mono-ol mono-carbonic acid of poly alkylene oxide (PAG) (PAG-MC) being reacted with itself;
b) at least one di-ol of poly alkylene oxide (PAG) (PAG-DO) with at least one PAG containing two carbonic acids as end-group, i.e. a carbonic acid-group at both ends of the PAG) (PAG-DC); or
c) at least one mono-ol mono-carbonic acid of poly alkylene oxide (PAG) (PAG-MC) with at least one di-ol of poly alkylene oxide (PAG) (PAG-DO) and at least one PAG containing two carbonic acids as end-group;
wherein optionally within each a), b) and c) at least one low molecular weight di-carbonic acids may be contained in addition to the compounds “PAG-DC”;
and
(B) polymeric sidechains grafted onto the polymer backbone A, wherein said polymeric sidechains (B) are obtainable by radical polymerization of at least one monomer selected from the group consisting of i) at least one vinyl ester monomer (B1) and ii) optionally at least one further olefinically unsaturated monomer (B2) polymerizable with monomer B1.

13. The graft polymer according to claim 12, wherein the polymer backbone is obtained from the reaction according to option c).

14. The graft polymer according to claim 10,
wherein the PAG of the PAG-DO, PAG-DC and PAG-MC may be independently selected from PAG-units consisting of one, two, three, four or more different alkylene oxide-monomers, and
wherein in case of more than one alkylene oxide monomer being comprised the structure of the PAG is a block copolymer, a random polymer, or a polymer comprising mixed structures of block units (with each block being a homo-block or a random block itself) and statistical/random parts comprised of two or more alkylene oxides,
wherein each PAG-unit can be a block polymer, a random polymer or a polymer of mixed block-random-structure, and
wherein in case more than one PAG-DO, PAG-DC and/or PAG-MC are employed each PAG-DO, PAG-DC and PAG-MC may be independently selected from those described structures of the PAG-units.

15. The graft polymer according to claim 1, comprising
more than 0.2% by weight of the polymeric sidechains (B) (in relation to the total weight of the graft polymer).

16. The graft polymer according to claim 1, comprising
20 to 95% by weight of the polymer backbone (A) and 5 to 80% by weight of the polymeric sidechains (B) (in relation to the total weight of the graft polymer).

17. The graft polymer according to claim 1, comprising
40 to 85% by weight, of the polymer backbone A.

18. The graft polymer according to claim 1,
with a weight average molecular weight Mw of from 500 to 500 000 g/mol.

19. The graft polymer according to claim 1,
having a polydispersity Mw/Mn of 1.2 to 6.

20. The graft polymer according to claim 1, wherein
the polymeric sidechains (B) comprise at least one vinyl ester monomer (B1) and optionally at least one olefinically unsaturated monomer (B2) other than the monomer (B1), and wherein the remaining amount of vinyl ester may be any other known vinyl ester.

21. The graft polymer according to claim 1, wherein
the optionally at least one olefinically unsaturated monomer (B2) is selected from the group consisting of monomers B2a and monomers B2b.

22. The graft polymer according to claim 1, wherein
the amount of vinyl ester monomer (B1) is from 1 to 100 by weight of at least one vinyl ester monomer (B1), and
the amount of the optional at least one further monomer (B2) is from 0 to 99%, by weight.

23. A method of obtaining at least one graft polymer according to claim 1, wherein
i) at least one vinyl ester monomer (B1) and
ii) optionally at least one further olefinically unsaturated monomer (B2) polymerizable with monomer B1
are polymerized by radical polymerization using suitable radical initiators in the presence of at least one polymer backbone (A).

24. The method according to claim 23, wherein
the amount of the at least one vinyl ester monomer (B1) is from 1 to 100% by weight, % by weight based on the total amount of monomers B, and wherein the remaining amount of vinyl ester may be any other known vinyl ester,
and
the optionally at least one olefinically unsaturated monomer (B2) is selected from the group consisting of monomers B2a and monomers B2b,
and wherein the amount of (B2) is from 0 to 99% by weight based on the total amount of monomers B.

25. The method according to claim 23, wherein the monomer B2 is a N-vinyllactams.

26. The method according to claim 23, comprising the polymerization of at least one monomer (B1) and the optionally at least one further monomer (B2) in order to obtain the polymer sidechains (B) in the presence of at least one polymer backbone (A), a free radical-forming initiator (C) and, if desired, up to 50% by weight, based on the sum of components (A), (B1), optionally (B2), and (C) of at least one organic solvent (D), at a mean polymerization temperature at which the initiator (C) has a decomposition half-life of from 40 to 500 min, in such a way that the fraction of unconverted grafted monomers (B1) and optionally (B2) and initiator (C) in the reaction mixture is constantly kept in a quantitative deficiency relative to the polymer backbone (A).

27. A method of using at least one graft polymer according to claim 1, the method comprising using the graft polymer
in a cleaning composition, fabric and home care product, industrial and institutional cleaning product, cosmetic or personal care product, oil field-formulation, crude oil emulsion breaker, pigment dispersion for ink jet inks, inks containing the graft polymer, electro plating product, cementitious composition, lacquer, paint, and/or agrochemical formulations.

28. The method according to claim 27, the method comprising using the graft polymer in cleaning compositions and/or in fabric and home care products, wherein the at least one graft polymer is present in the cleaning composition at a concentration of from about 0.1% to about 20%, each in weight % in relation to the total weight of the cleaning compositions and/or the fabric and home care products.

29. (canceled)

30. (canceled)

31. (canceled)

32. A cleaning composition or fabric and home care product comprising at least one graft polymer according to claim 1.

33. (canceled)

34. (canceled)

35. (canceled)

36. (canceled)

37. (canceled)

38. An agrochemical composition comprising at least one graft polymer according to claim 1, and at least one agrochemical active ingredient.

39. (canceled)

40. A method for controlling phytopathogenic fungi and/or undesired plant growth and/or undesired attack by insects or mites and/or for regulating the growth of plants, where the agrochemical composition as defined in claim 38 is allowed to act on the particular pests, their habitat or the plants to be protected from the particular pest, the soil and/or on undesired plants and/or the useful plants and/or their habitat.

41. A method for combating or controlling invertebrate pests, which method comprises contacting said pest or its food supply, habitat or breeding grounds with a pesticidally effective amount of the agrochemical composition as defined in claim 38.

42. A method for protecting growing plants from attack or infestation by invertebrate pests, which method comprises contacting a plant, or soil or water in which the plant is growing, with a pesticidally effective amount of the agrochemical composition as defined in claim 38.

43. A seed comprising the agrochemical composition, as defined in claim 38, in an amount of from 0.1 g to 10 kg per 100 kg of seed.

44. A method for treating or protecting an animal from infestation or infection by invertebrate pests, which comprises bringing the animal in contact with a pesticidally effective amount of the agrochemical composition as defined in claim 38.
